(19) United States
(12) Patent Application Publication
(10) Pub. No.: US 2026/0285824 A1
(43) Pub. Date: Sep. 24, 2026

(54) BUTENOLIDE-BASED ESTROGEN RECEPTOR ALPHA INHIBITORS AND DEGRADERS AND METHOD OF USING THE SAME

(71) Applicant: Georgia Tech Research Corporation, Atlanta, GA (US)

(72) Inventors: Adegboyega Oyelere, Atlanta, GA (US); Dipak Walunj, Atlanta, GA (US); Alexis Johnston, Atlanta, GA (US); Jeremiah Olugbami, Atlanta, GA (US)

(21) Appl. No.: 19/565,396

(22) Filed: Mar. 12, 2026

Related U.S. Application Data
(60) Provisional application No. 63/770,605, filed on Mar. 12, 2025.

(51) Int. Cl. C07D 307/58; A61K 31/365; A61K 31/4025; A61K 31/4192; A61K 31/443; A61K 31/496; A61P 35/00; C07D 405/04; C07D 405/10

(52) U.S. Cl. CPC C07D 307/58; A61K 31/365; A61K 31/4025; A61K 31/4192; A61K 31/443; A61K 31/496; A61P 35/00; C07D 405/04; C07D 405/10

(57) ABSTRACT

An exemplary embodiment of the present disclosure provides a compound of formula (I) having the following structure:

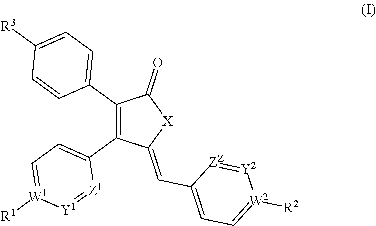

or a stereoisomer, pharmaceutically acceptable salt, oxide, or solvate thereof, wherein R¹, R², R³, X, W¹, Y¹, Z¹, W², Y², Z² are as described herein. The present disclosure also provides compositions comprising these compounds and methods of using these compounds to inhibit or degrade an estrogen receptor alpha, particularly for the treatment of cancer.

### CROSS-REFERENCE TO RELATED APPLICATIONS

This application claims the benefit of U.S. Provisional Application No. 63/770,605, filed 12 Mar. 2025, which is incorporated herein by reference in its entirety as if fully set forth below.

### GOVERNMENT LICENSE RIGHTS

This invention was made with government support under CA252720 awarded by National Institutes of Health. The government has certain rights in the invention.

### FIELD OF THE DISCLOSURE

The various embodiments of the present disclosure relate generally to a composition of matter comprising butenolide-based small molecule inhibitors and degraders of estrogen receptor alpha and the method of their use as anti-cancer agents for cancers that depend on ERa, RET, GREB1, ADCY5 and TFF1, including breast, prostate, lung and liver cancers.

### BACKGROUND

Breast cancer (BCa) is second only to lung cancer as the leading cause of cancer deaths in US women. In 2025, about 316,950 new cases of invasive BCa are expected to be diagnosed in women and 2,800 cases in men in the US; of these, 42,680 will succumb to this disease [ref: American Cancer Society. Cancer Facts & Figures 2025. Atlanta: American Cancer Society; 2025.]. Drugs that target ER signaling malfunctions, a driver of more than 70% of BCas, have demonstrable success in BCa therapy and/or chemo-prevention. For example, selective estrogen-receptor modulators (SERMs) such as tamoxifen (Tam) and selective estrogen receptor downregulator (SERD) fulvestrant (Ful) are standard therapies for the treatment of hormone-dependent BCa. Other SERDs are in development and Elacestrant is the most recent to obtain FDA approval [ref: Lai A C, Crews C M. Induced protein degradation: an emerging drug discovery paradigm. Nat Rev Drug Discov. 2017, 16, 101-114. doi: 10.1038/nrd.2016.211.]. Despite the initial benefits, patients eventually relapse due to acquired resistance to most of these drugs. Butenolides are a class of lactone that are relatively more stable than their corresponding g-butyrolactones, which have elicited several biological activities [ref:(a) Kapoor et al. eLife 2020; 9:e57824. DOI: https://doi.org/10.7554/eLife.57824; (b) Zhang Y F, Zhang H, He L, Liu C, Xu Y, Qian P Y. Butenolide inhibits marine fouling by altering the primary metabolism of three target organisms. ACS Chem Biol. 2012; 7(6):1049-1058. doi:10.1021/cb200545s; (c) Tabei Y, Murotomi K, Umeno A, et al. Antioxidant properties of 5-hydroxy-4-phenyl-butenolide via activation of Nrf2/ARE signaling pathway. Food Chem Toxicol. 2017; 107(Pt A):129-137. doi:10.1016/j.fct.2017.06.039; (d) Y.-M. Wang et al. Toxicology Letters 164 (2006) 231-238; (e) Woelflingseder L, Adam G and Marko D (2020) Suppression of Trichothecene-Mediated Immune Response by the Fusarium Secondary Metabolite Butenolide in Human Colon Epithelial Cells. Front. Nutr. 7:127. doi: 10.3389/fnut.2020.00127; (f) WO2014027826A1; (g) Xie L, Li R, Zheng B, et al. One-Step Transformation from Rofecoxib to a COX-2 NIR Probe for Human Cancer Tissue/Organoid Targeted Bioimaging. ACS Appl Bio Mater. 2021; 4(3):2723-2731. doi:10.1021/acsabm.0c01634.]

Accordingly, there is a need for improved compositions and methods for inhibiting and degrading estrogen receptor alpha. Embodiments of the present disclosure are directed to this and other considerations.

### BRIEF SUMMARY

An exemplary embodiment of the present disclosure provides a compound of formula (I) having the following structure:

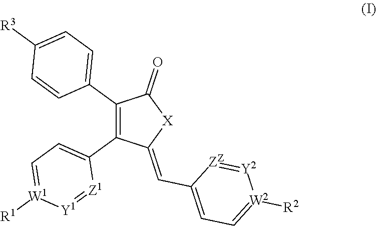

or a stereoisomer, pharmaceutically acceptable salt, oxide, or solvate thereof, wherein
   R¹ is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶;
   R² is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶
   R³ is H, hydroxyl, —SO₂C₁₋₆ alkyl, —NR⁴R⁵, —CN, heterocyclyl, aryl, heteroaryl, —CH₂R⁶, —OR⁶ or —SR⁶
   X is O or NR⁷;
   W¹ is C or N, wherein when W¹ is N, R¹ is not present;
   Y¹ is CH or N;
   Z¹ is CH or N;
   W² is C or N, wherein when W² is N, R² is not present;
   Y² is CH or N;
   Z² is CH or N;
   R⁴ is H, C₁-C₆ alkyl, C₂-C₆ alkenyl, wherein C₁-C₆ alkyl and C₂-C₆ alkenyl can be substituted with —N(CH₃)₂ or heterocyclyl;
   R⁵ is H or C₁-C₆ alkyl; or
   R⁴ and R⁵ combine with the nitrogen atom to which they are attached to form a heterocyclic ring, wherein the heterocyclic ring can be substituted with C₁-C₆ alkyl;
   R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and
   R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.

In any of the embodiments disclosed herein, the compound of formula (I) can be selected from the group consisting of

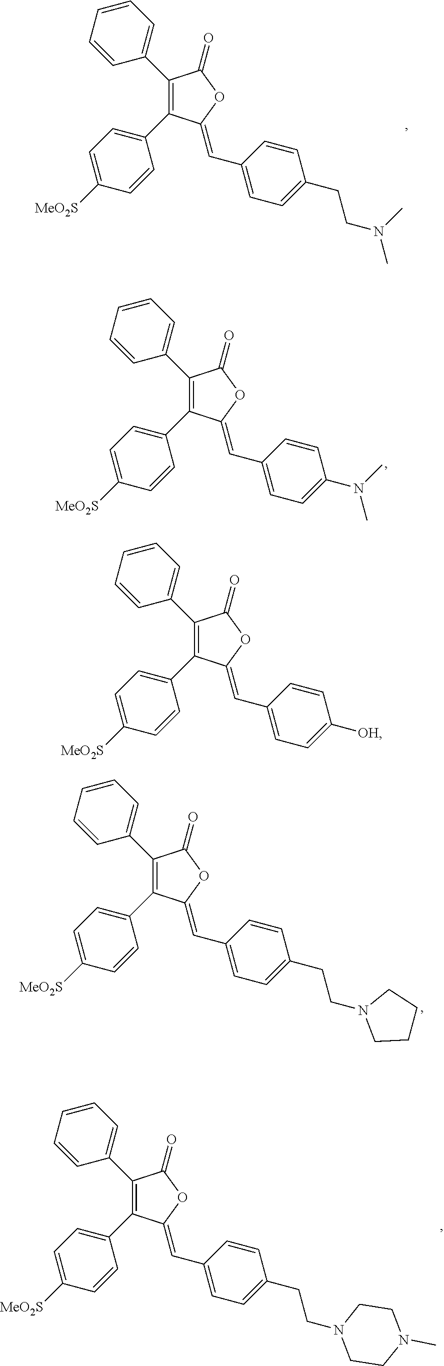

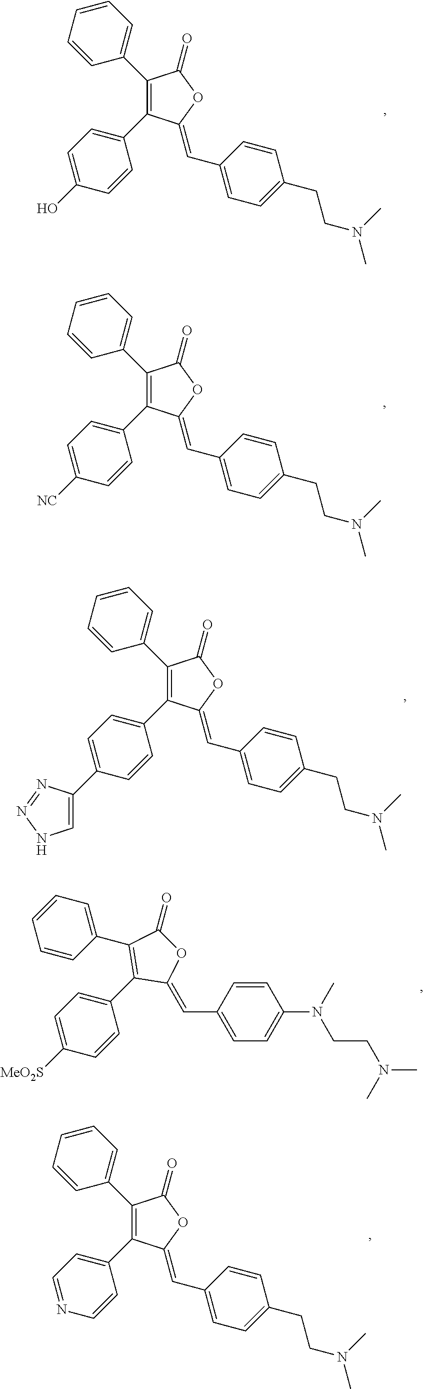 [SMILES: CN(C)CCc1ccc(/C=C2\OC(=O)C(c3ccccc3)=C2c2ccc(O)cc2)cc1 ; CN(C)CCc1ccc(/C=C2\OC(=O)C(c3ccccc3)=C2c2ccc(C#N)cc2)cc1 ; CN(C)CCc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCc1ccc(/C=C2\OC(=O)C(c3ccccc3)=C2c2ccncc2)cc1]

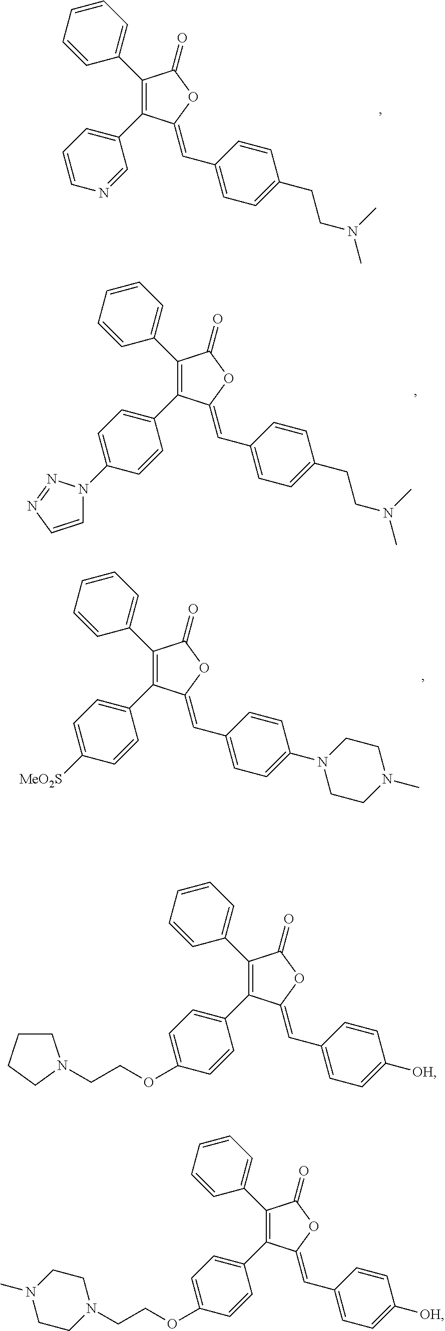

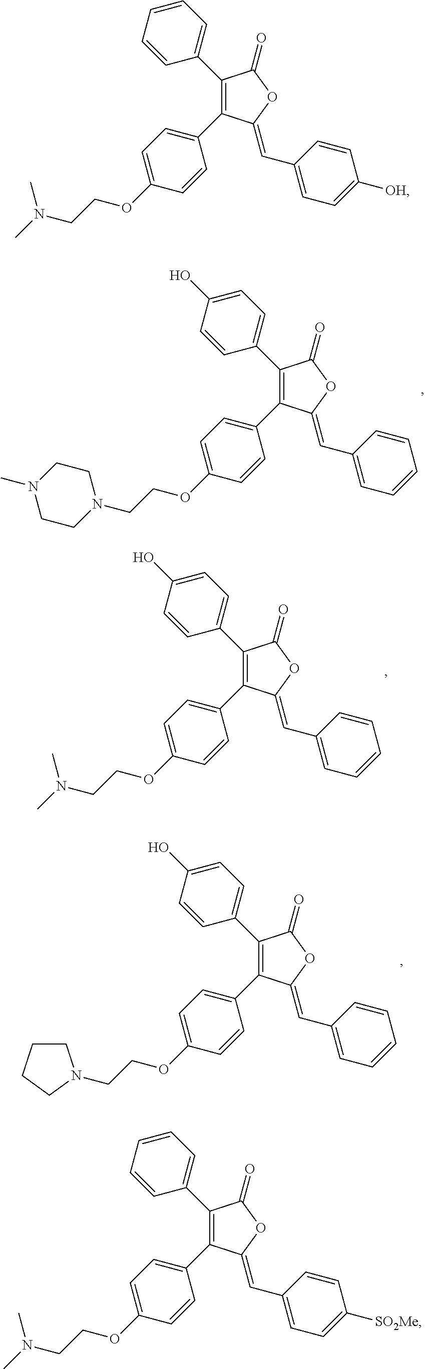 [SMILES: CN(C)CCOc1ccc(C2=C(c3ccccc3)C(=O)O/C2=C\c2ccc(O)cc2)cc1 ; CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1]

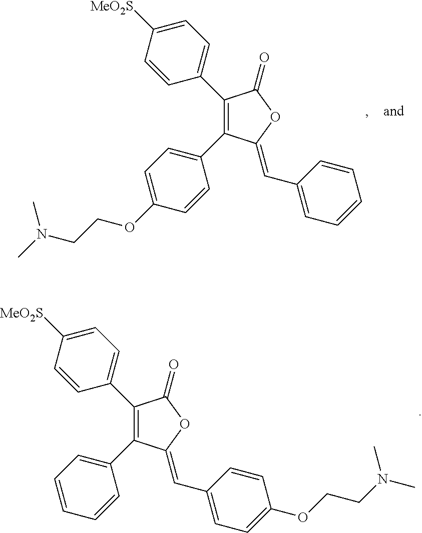 [SMILES: CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

An exemplary embodiment of the present disclosure provides a compound of formula (I) having the following structure:

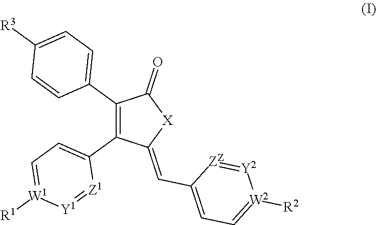

or a stereoisomer, pharmaceutically acceptable salt, oxide, or solvate thereof, wherein
   R¹ is optionally present, and when present is H, hydroxyl, —SO₂C₁₋₆ alkyl, —NR⁴R⁵, —CN, heterocyclyl, aryl, heteroaryl, —OR⁶ or —SR⁶;
   R² is optionally present, and when present is H, hydroxyl, —SO₂C₁₋₆ alkyl, —NR⁴R⁵, —CN, heterocyclyl, aryl, heteroaryl, —OR⁶ or —SR⁶;
   R³ is H, hydroxyl, —SO₂C₁₋₆ alkyl, —NR⁴R⁵, —CN, heterocyclyl, aryl, heteroaryl, —OR⁶ or —SR⁶;
   X is O or NR⁷;
   W¹ is C or N, wherein when W¹ is N, R¹ is not present;
   Y¹ is CH or N;
   Z¹ is CH or N;
   W² is C or N, wherein when W² is N, R² is not present;
   Y² is CH or N;
   Z² is CH or N;
   R⁴ is H, C₁-C₆ alkyl, C₂-C₆ alkenyl, wherein C₁-C₆ alkyl and C₂-C₆ alkenyl can be substituted with —N(CH₃)₂ or heterocyclyl;
   R⁵ is H or C₁-C₆ alkyl; or
   R⁴ and R⁵ combine with the nitrogen atom to which they are attached to form a heterocyclic ring, wherein the heterocyclic ring can be substituted with C₁-C₆ alkyl;
   R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and
   R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.

In any of the embodiments disclosed herein, the compound of formula (I) can be selected from the group consisting of

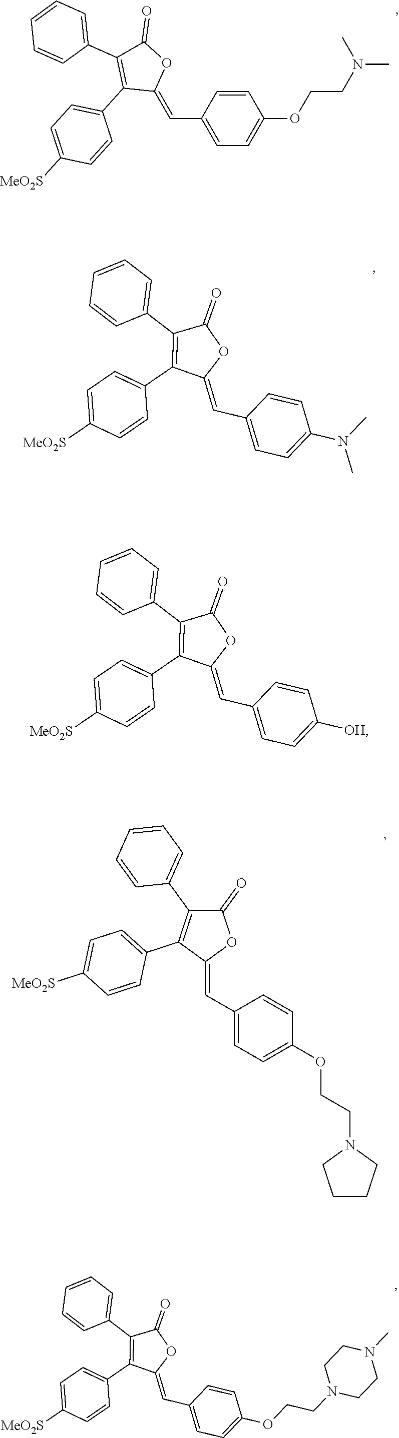 [SMILES: CN(C)CCOc1ccc(/C=C2\OC(=O)C(c3ccccc3)=C2c2ccc(S(C)(=O)=O)cc2)cc1 ; CN(C)c1ccc(/C=C2\OC(=O)C(c3ccccc3)=C2c2ccc(S(C)(=O)=O)cc2)cc1 ; CS(=O)(=O)c1ccc(C2=C(c3ccccc3)C(=O)O/C2=C\c2ccc(O)cc2)cc1 ; CS(=O)(=O)c1ccc(C2=C(c3ccccc3)C(=O)O/C2=C\c2ccc(OCCN3CCCC3)cc2)cc1 ; CN1CCN(CCOc2ccc(/C=C3\OC(=O)C(c4ccccc4)=C3c3ccc(S(C)(=O)=O)cc3)cc2)CC1]

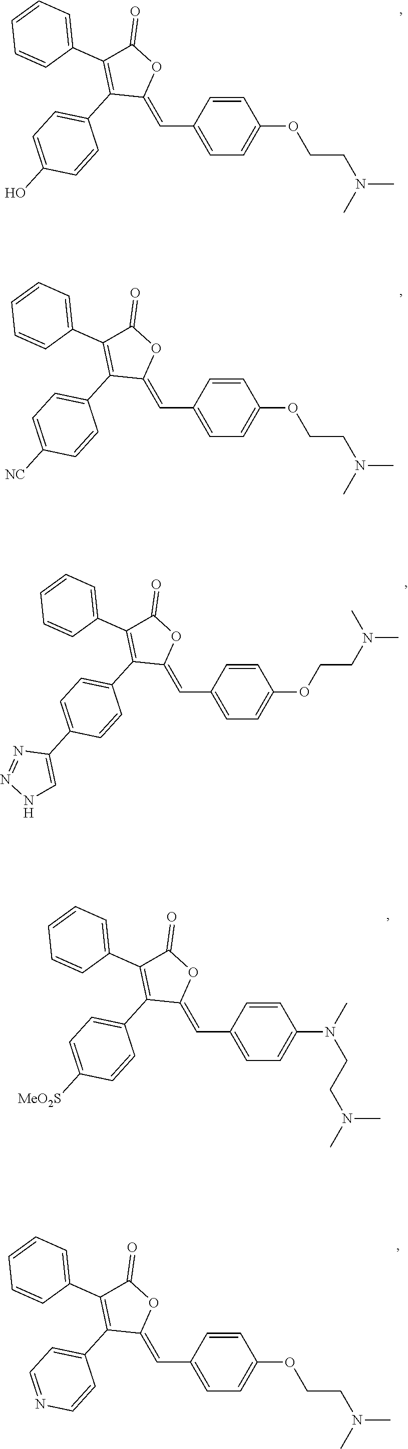 [SMILES: CN(C)CCOc1ccc(/C=C2\OC(=O)C(c3ccccc3)=C2c2ccc(O)cc2)cc1 ; CN(C)CCOc1ccc(/C=C2\OC(=O)C(c3ccccc3)=C2c2ccc(C#N)cc2)cc1 ; CN(C)CCOc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCOc1ccc(/C=C2\OC(=O)C(c3ccccc3)=C2c2ccncc2)cc1]

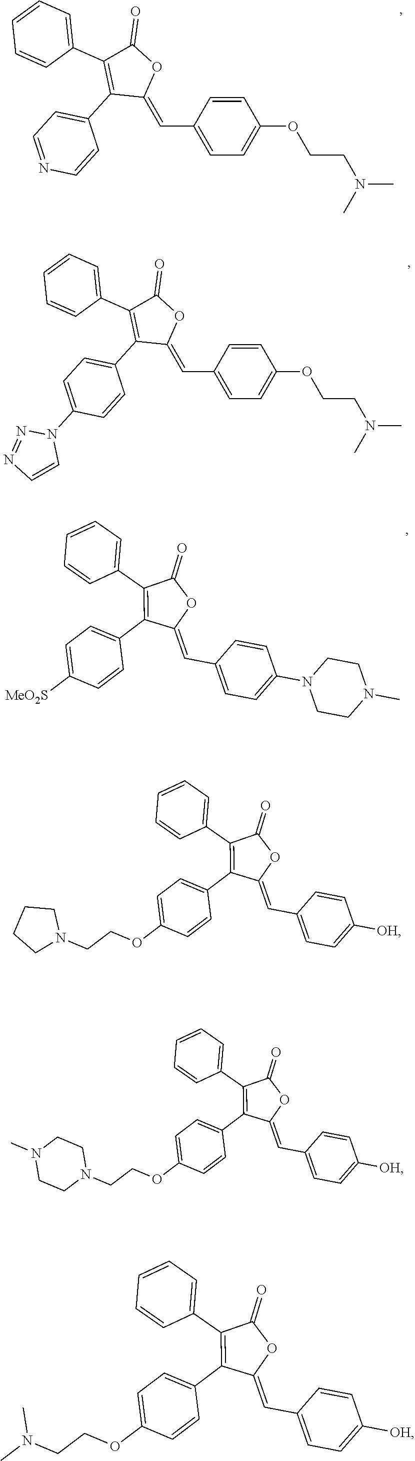

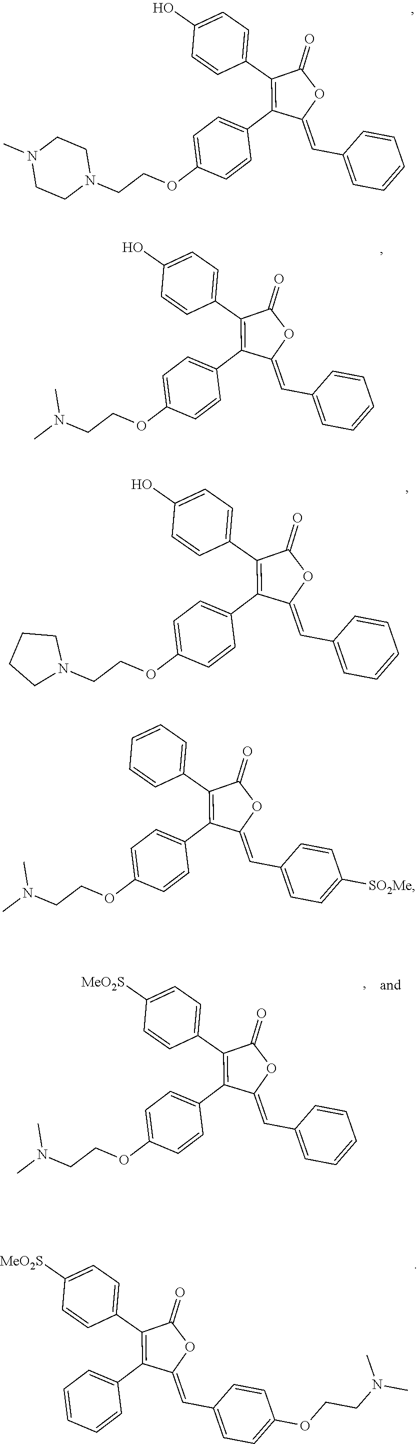 [SMILES: CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1 ; CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

Another exemplary embodiment of the present disclosure provides a method of treating cancer in a subject. The method can comprise providing a compound as disclosed herein to a subject in need of said treatment.

In any of the embodiments disclosed herein, the cancer can be an estrogen receptor (ER) dependent cancer (i.e., the cancer can depend on estrogen receptor (ER) signaling).

In any of the embodiments disclosed herein, the cancer can be selected from the group consisting of breast cancer, prostate cancer, lung cancer, and liver cancer.

Another exemplary embodiment of the present disclosure provides a method of inhibiting an estrogen receptor alpha. The method can comprise providing a compound as disclosed herein under conditions effective to inhibit the estrogen receptor alpha (ERa).

Another exemplary embodiment of the present disclosure provides a method of degrading an estrogen receptor alpha. The method can comprise providing a compound as disclosed herein under conditions effective to degrade the estrogen receptor alpha (ERα).

Another exemplary embodiment of the present disclosure provides a method of treating a hyperproliferative disorder. The method can comprise providing a compound as disclosed herein under conditions effective to treat a hyperproliferative disorder.

In any of the embodiments disclosed herein, the hyperproliferative disorder can depend on ERa, RET, GREB1, ADCY5 or TFF1.

In any of the embodiments disclosed herein, the hyperproliferative disorder can be a cancer selected from the group consisting of breast cancer, prostate cancer, lung cancer, and liver cancer.

Another exemplary embodiment of the present disclosure provides a composition comprising a compound as disclosed herein and a carrier.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides concentration-response curves of an estrogen receptor alpha (ERα)-binding activity of tamoxifen (Tam, standard) and butenolide-based compounds, in accordance with an exemplary embodiment of the present disclosure. Two sets of compounds are evident from the curves; both compounds DW-165 and DW-178 do not exhibit affinity for ERα, whereas compounds DW-380, DW-427, DW-435, and DW-438, along with the reference compound (tamoxifen), display varying binding affinities for Era.

FIGS. 2A-2F provide assessment of anti-replicative activity of butenolide-based compounds using a clonogenic assay, in accordance with an exemplary embodiment of the present disclosure. For these assays, MDA-MB-231 cells were treated for 72 h with the respective concentrations of ½×IC₅₀, IC₅₀, and 2×IC₅₀ values of each compound. FIG. 2A shows the colony status of the controls which consist of tamoxifen (Tam, standard) and dimethyl sulfoxide (DMSO). FIG. 2B shows the colony status of compound DW-165. FIG. 2C shows the colony status of compound DW-271. FIG. 2D provides the quantifications of the colony status of the controls. FIG. 2E provides the quantifications of the colony status of compound DW-165. FIG. 2F provides the quantifications of the colony status of compound DW-271. For all quantification, **P<0.05; **P<0.001; ****P<0.0001.

FIGS. 3A-3B provide western blot analysis of MDA-MB-231 cells treated with butanolide-based compounds for 24 h, in accordance with an exemplary embodiment of the present disclosure. FIG. 3A depicts the western blot electrophoretogram of total cell extracts from treated MDA-MB-231 cells. FIG. 3B provides the quantification of p21 expression levels by densitometry which shows the modulatory effects of the compounds on the levels of p21.

FIGS. 4A-4B show RNA seq revealed that DW-165 caused significant (p-value <0.05, false discovery rate <0.25) negative enrichment of the hallmark estrogen response early and estrogen response late MSigDB gene sets, in accordance with an exemplary embodiment of the present disclosure. FIG. 4A depicts a heatmap showing normalized enrichment scores. FIG. 4B provides a table reporting the normalized enrichment scores following RNA seq analysis. [symbol] hat DW-165 downregulates ESR1 and estrogen-responsive genes containing estrogen receptor binding sites, in accordance with an exemplary embodiment of the present disclosure. FIG. 5A provides a heatmap showing log 2 fold change values. FIG. 5B depicts a log 2 fold change table where the values are quantitatively represented.

FIGS. 6A-6B show RNA seq revealed that DW-165 downregulates transcription factors, GATA3, TFAP2C, and FOXO3, involved in estrogen signaling, in accordance with an exemplary embodiment of the present disclosure. FIG. 6A depicts a heat map of log 2 fold change values. FIG. 6B provides a log 2 fold change table where the values are quantitatively represented.

FIGS. 7A-7B show RNA seq revealed that DW-165 downregulates genes in the estrogen signaling pathway via nuclear-initiated steroid signaling, in accordance with an exemplary embodiment of the present disclosure. FIG. 7A provides a heat map of log 2 fold change values. FIG. 7B depicts a log 2 fold change table where the values are quantitatively represented.

FIGS. 8A-8B show RNA seq revealed that DW-165 downregulates genes in the estrogen signaling pathway via membrane-initiated steroid signaling (part I), in accordance with an exemplary embodiment of the present disclosure. FIG. 8A depicts a heat map of log 2 fold change values. FIG. 8B provides a log 2 fold change table where the values are quantitatively represented.

FIGS. 9A-9B show RNA seq revealed that DW-165 downregulates genes in the estrogen signaling pathway via membrane-initiated steroid signaling (part II), in accordance with an exemplary embodiment of the present disclosure. FIG. 9A shows a heat map of log 2 fold change values. FIG. 9B depicts a log 2 fold change table where the values are quantitatively represented.

FIGS. 10A-10B show RNA seq revealed that DW-165 downregulates genes in the estrogen signaling pathway via membrane-initiated steroid signaling (part III), in accordance with an exemplary embodiment of the present disclosure. FIG. 10A shows a heat map of log 2 fold change values. FIG. 10B provides a log 2 fold change table where the values are quantitatively represented.

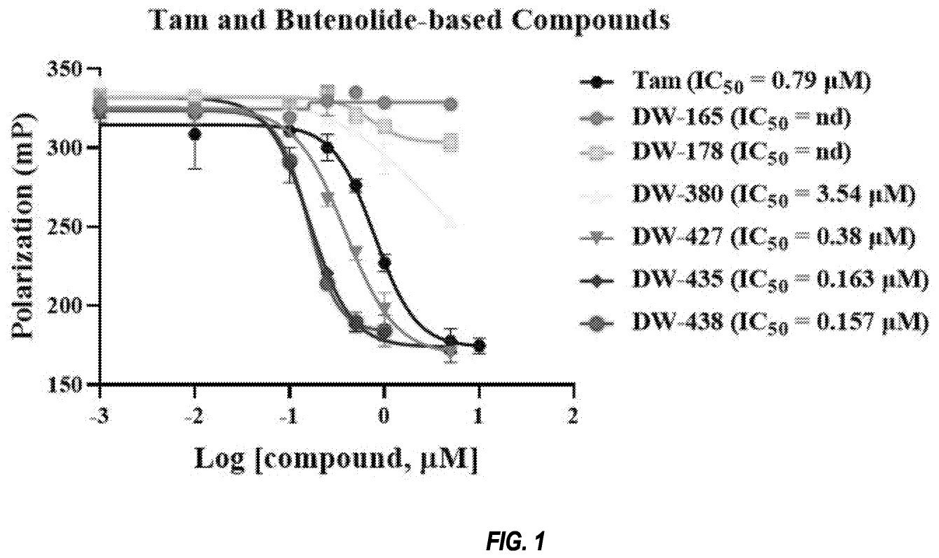

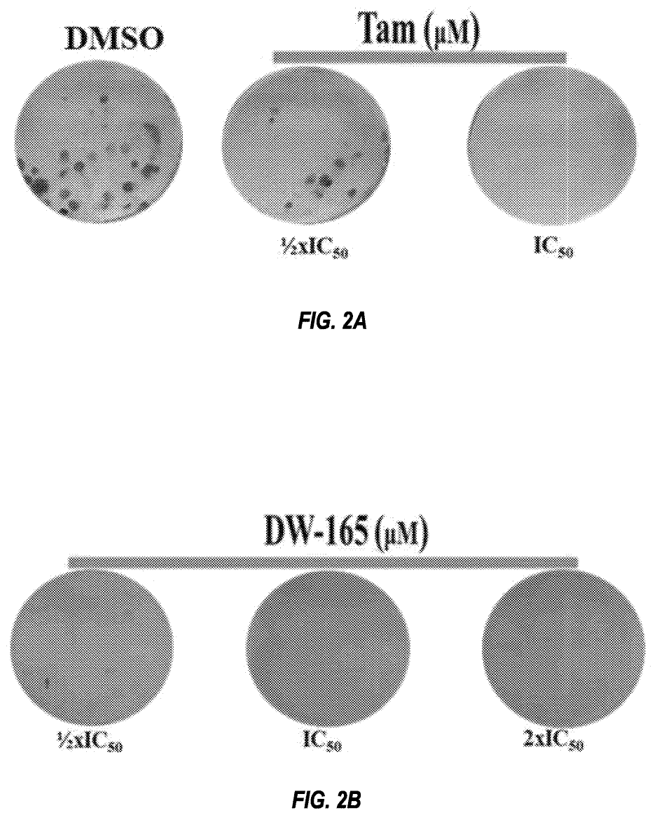

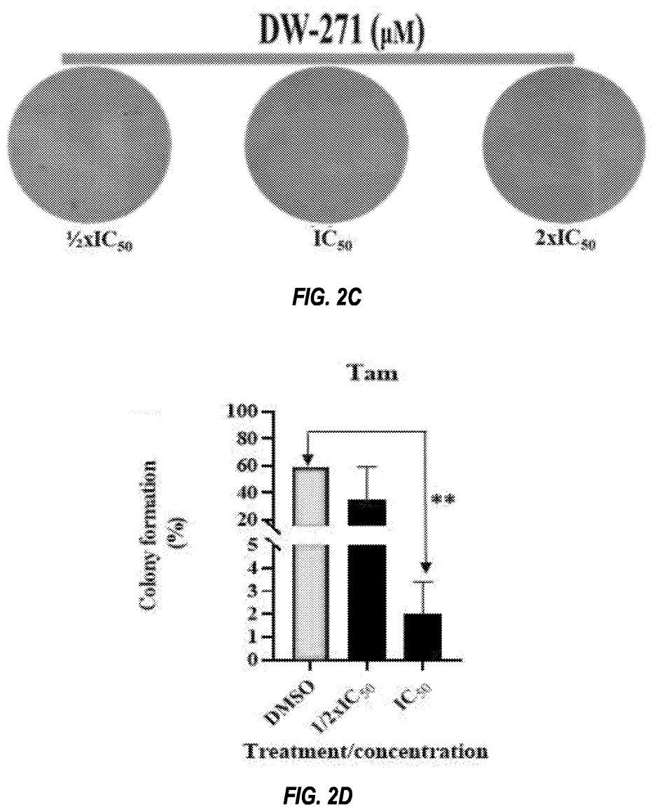

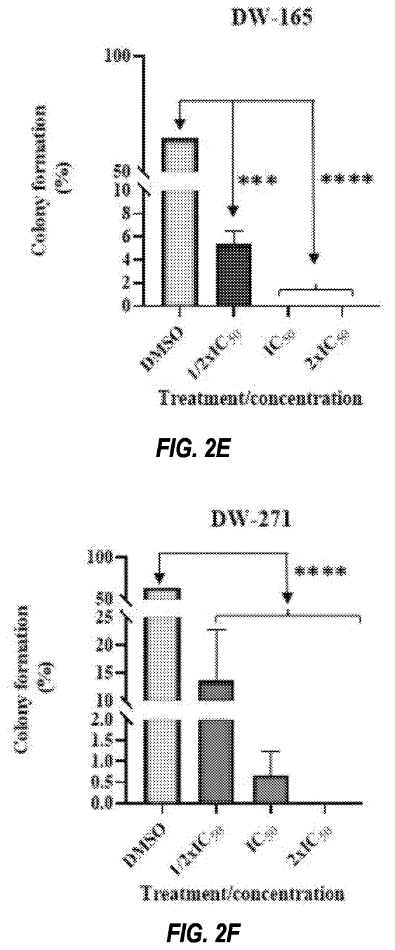

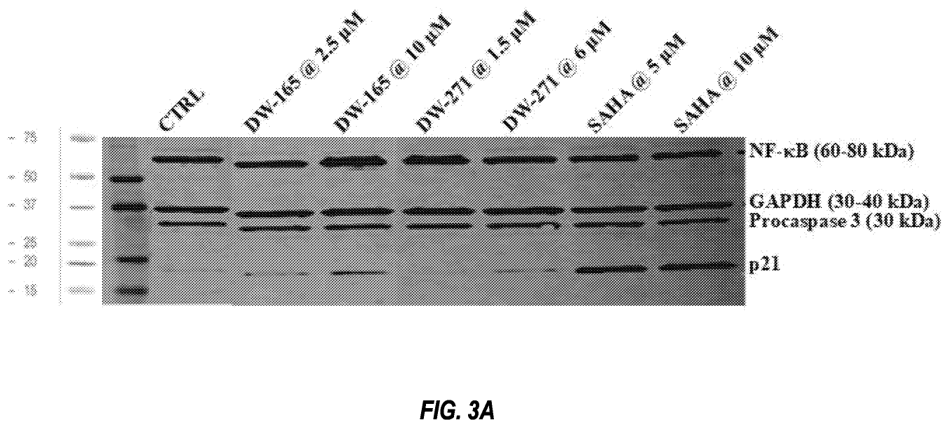

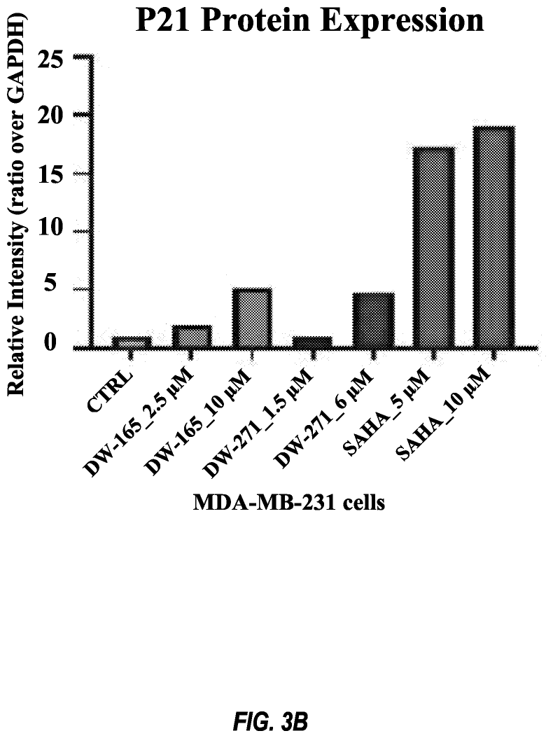

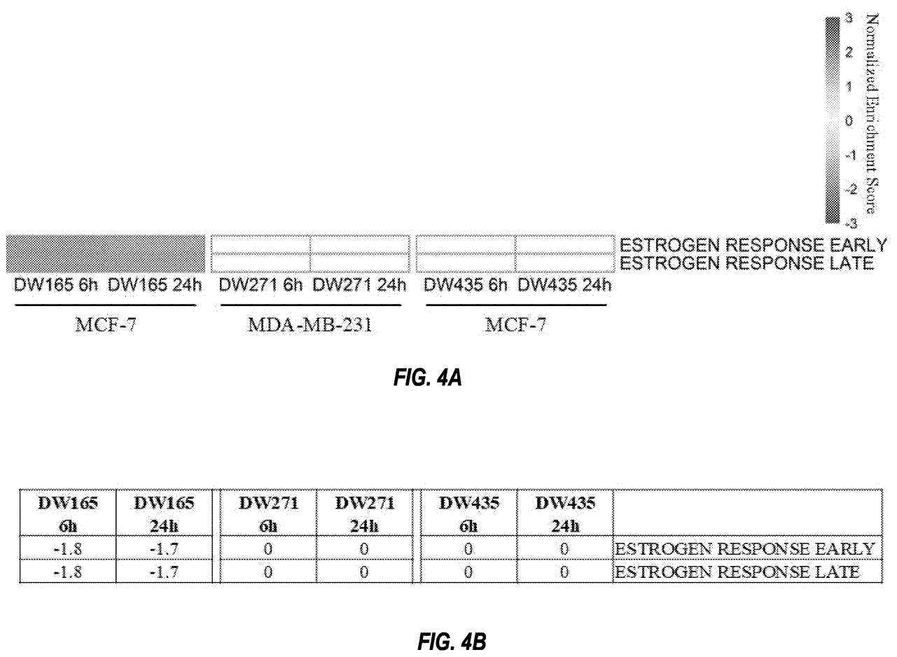

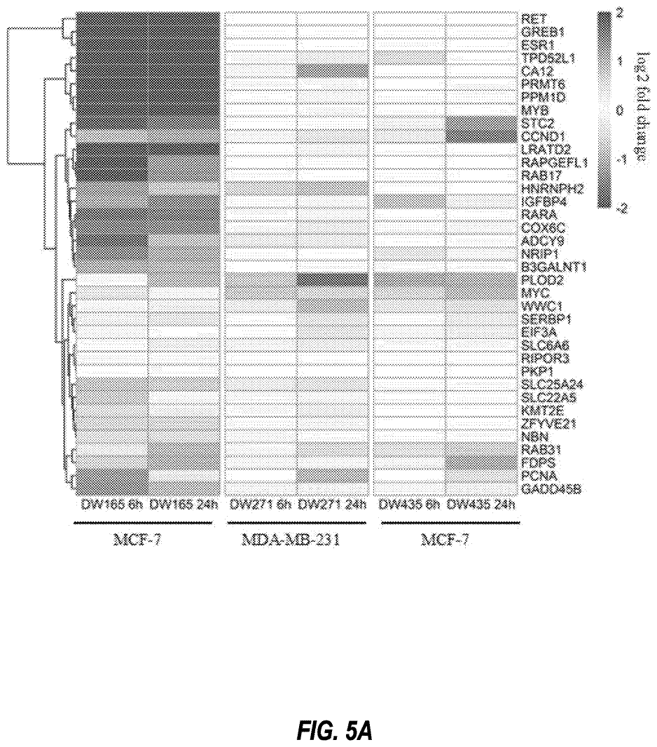

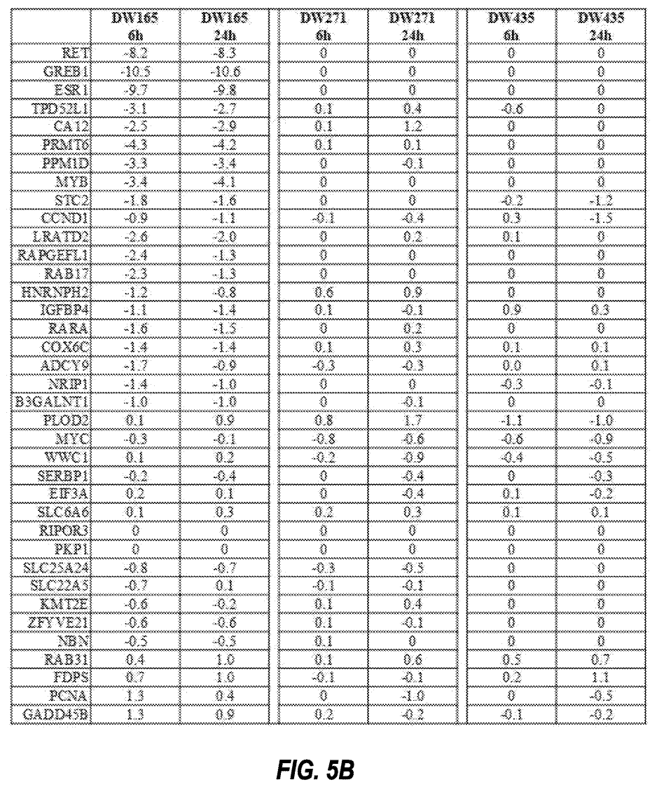

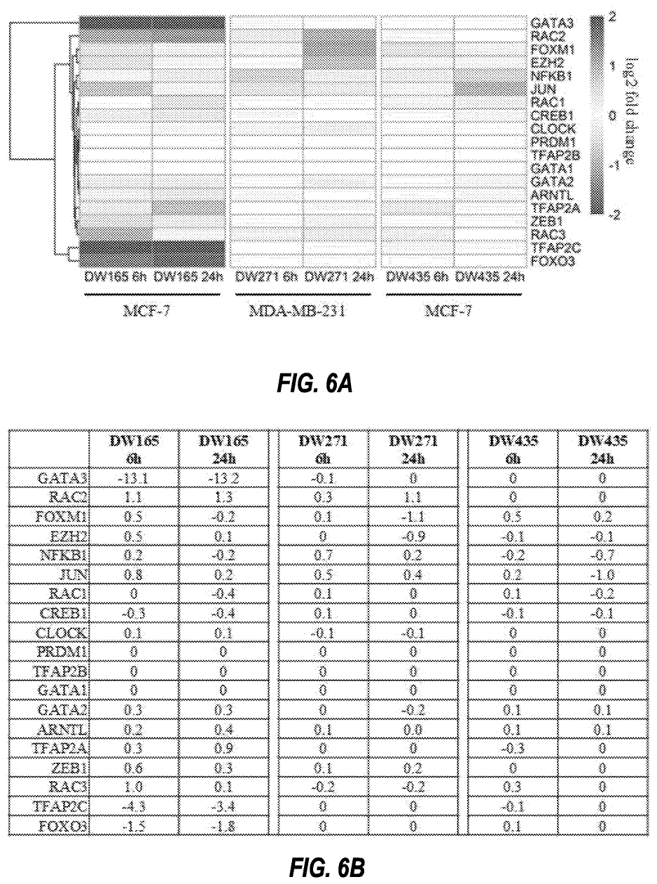

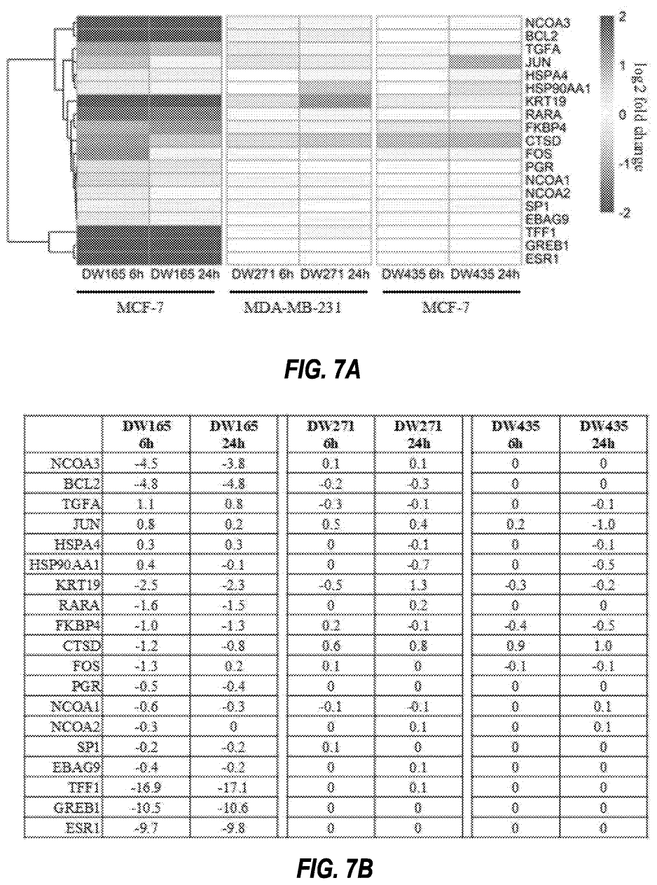

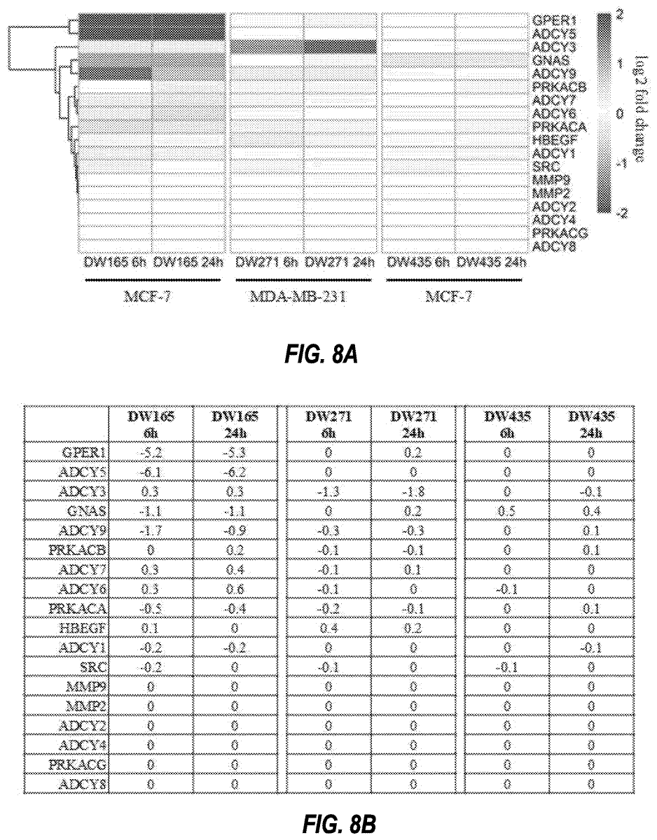

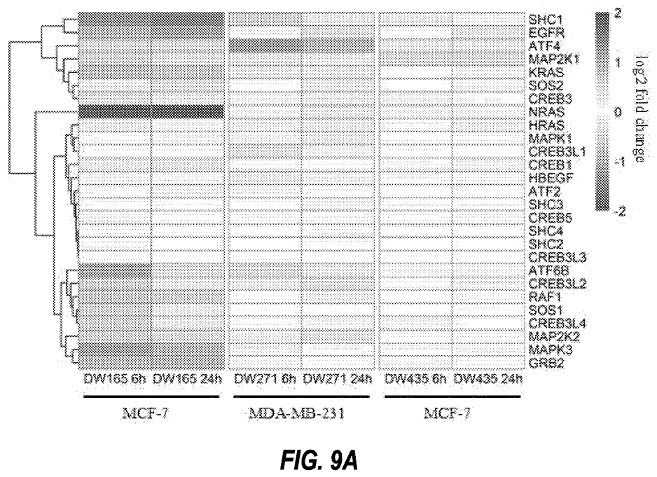

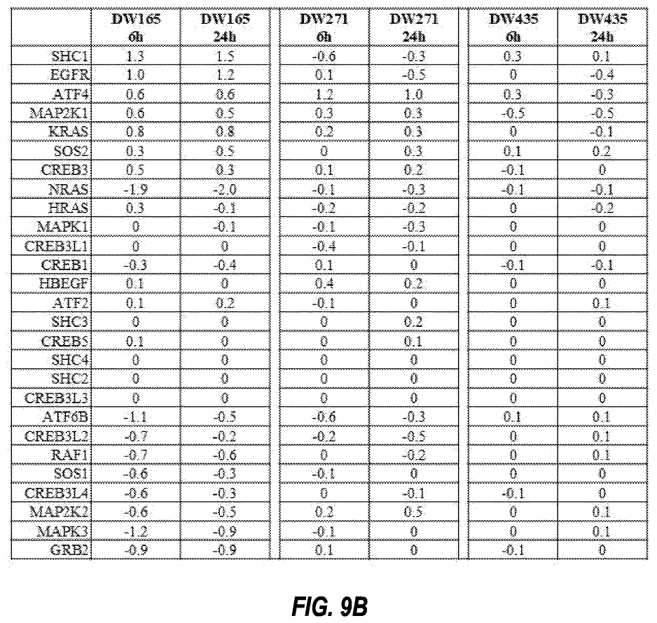

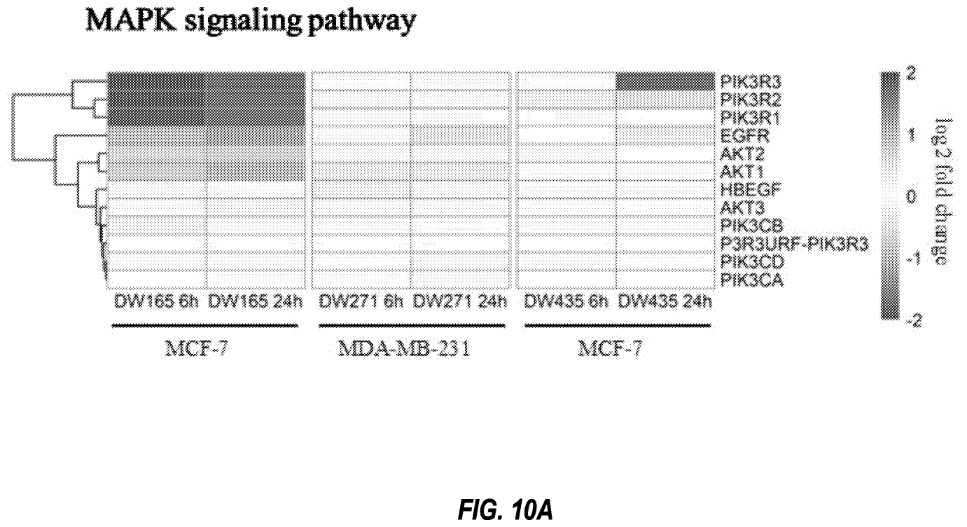

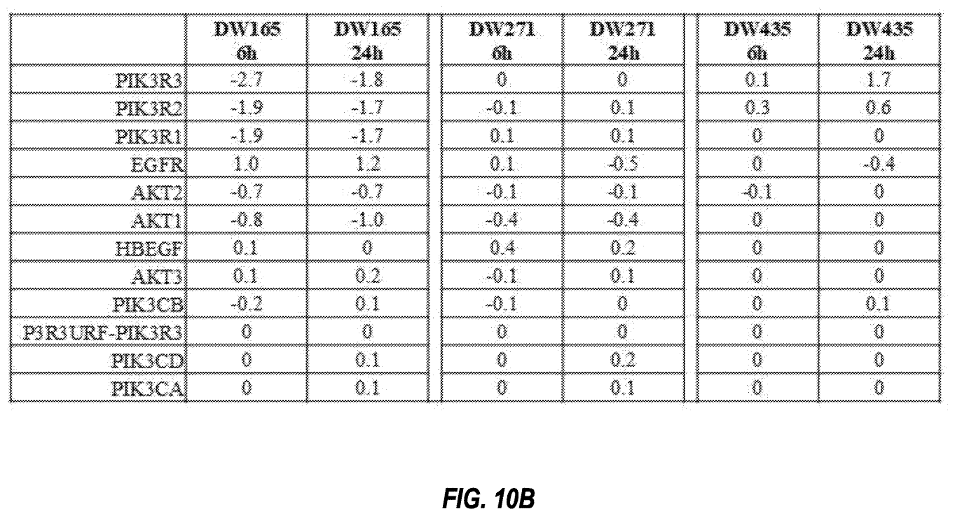

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

The present disclosure provides a composition of matter comprising butenolide-based small molecule estrogen receptor alpha (ERα) inhibitors. Another embodiment of the invention describes a composition of matter comprising butenolide-based small molecule ERa degraders. The disclosure also provides methods of using the disclosed composition of matter as therapy for hyperproliferative disorders that depend on ERα, RET, GREB1, ADCY5 and TFF1, including breast, prostate, lung and liver cancers.

An exemplary embodiment of the present disclosure provides a compound of formula (I) having the following structure:

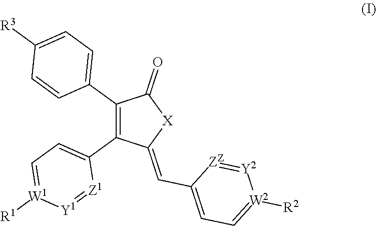

or a stereoisomer, pharmaceutically acceptable salt, oxide, or solvate thereof, wherein
   R¹ is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶
   R² is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶
   R³ is H, hydroxyl, —SO₂C₁₋₆ alkyl, —NR⁴R⁵, —CN, heterocyclyl, aryl, heteroaryl, —CH₂R⁶, —OR⁶ or —SR⁶;
   X is O or NR⁷;
   W¹ is C or N, wherein when W¹ is N, R¹ is not present;
   Y¹ is CH or N;
   Z¹ is CH or N;
   W² is C or N, wherein when W² is N, R² is not present;
   Y² is CH or N;
   Z² is CH or N;
   R⁴ is H, C₁-C₆ alkyl, C₂-C₆ alkenyl, wherein C₁-C₆ alkyl and C₂-C₆ alkenyl can be substituted with —N(CH₃)₂ or heterocyclyl;
   R⁵ is H or C₁-C₆ alkyl; or
   R⁴ and R⁵ combine with the nitrogen atom to which they are attached to form a heterocyclic ring, wherein the heterocyclic ring can be substituted with C₁-C₆ alkyl;
   R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and
   R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.

In some embodiments disclosed herein, the compound of formula (I) can be selected from the group consisting of

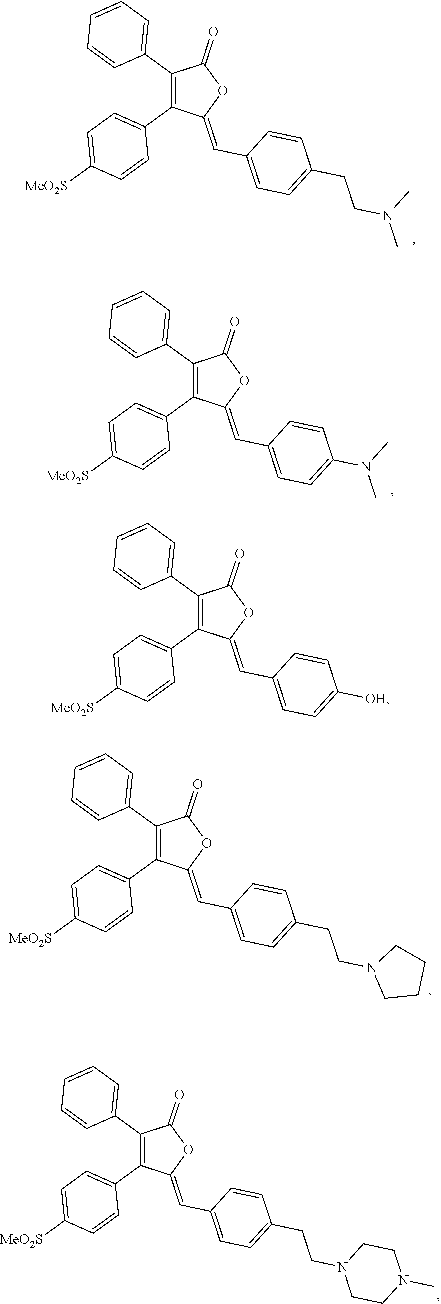 [SMILES: CN(C)CCc1ccc(/C=C2\OC(=O)C(c3ccccc3)=C2c2ccc(S(C)(=O)=O)cc2)cc1 ; CN(C)c1ccc(/C=C2\OC(=O)C(c3ccccc3)=C2c2ccc(S(C)(=O)=O)cc2)cc1 ; CS(=O)(=O)c1ccc(C2=C(c3ccccc3)C(=O)O/C2=C\c2ccc(O)cc2)cc1 ; CS(=O)(=O)c1ccc(C2=C(c3ccccc3)C(=O)O/C2=C\c2ccc(CCN3CCCC3)cc2)cc1 ; CN1CCN(CCc2ccc(/C=C3\OC(=O)C(c4ccccc4)=C3c3ccc(S(C)(=O)=O)cc3)cc2)CC1]

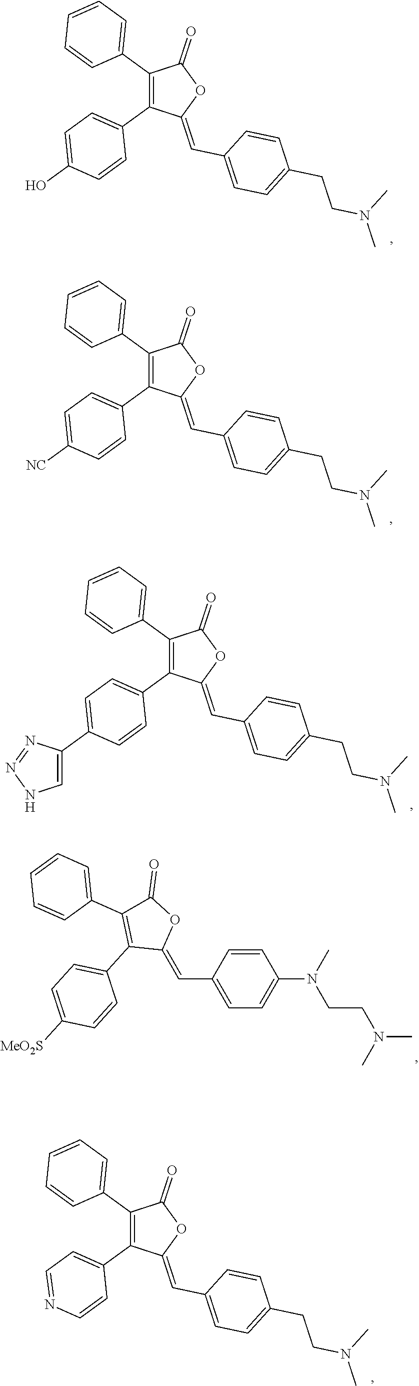 [SMILES: CN(C)CCc1ccc(/C=C2\OC(=O)C(c3ccccc3)=C2c2ccc(O)cc2)cc1 ; CN(C)CCc1ccc(/C=C2\OC(=O)C(c3ccccc3)=C2c2ccc(C#N)cc2)cc1 ; CN(C)CCc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCc1ccc(/C=C2\OC(=O)C(c3ccccc3)=C2c2ccncc2)cc1]

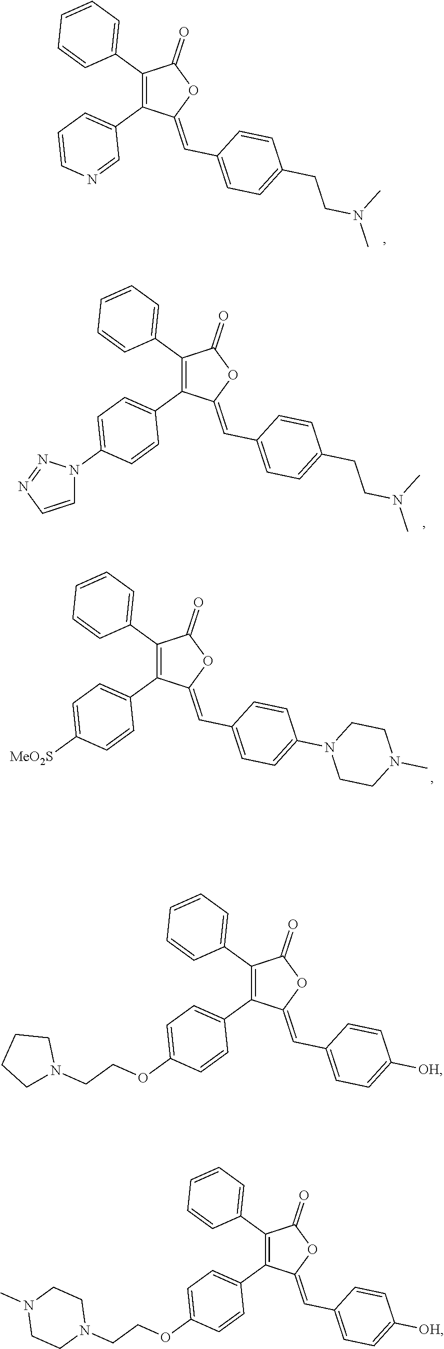

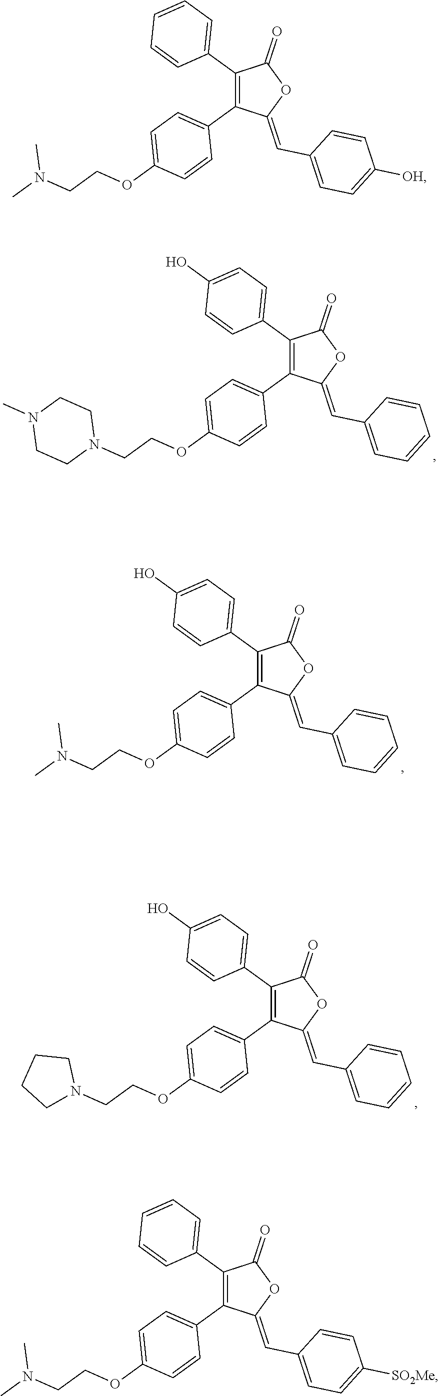 [SMILES: CN(C)CCOc1ccc(C2=C(c3ccccc3)C(=O)O/C2=C\c2ccc(O)cc2)cc1 ; CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1]

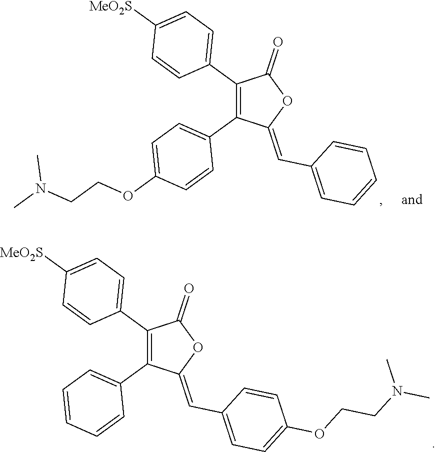 [SMILES: CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

Another exemplary embodiment of the present disclosure provides a compound of Formula II or Formula III:

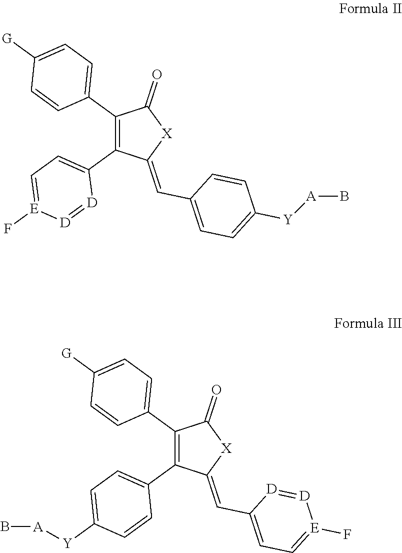

wherein
   X is O or NR;
   R is independently H, C₁₋₆ alkyl, optionally substituted with unsaturation and heteroatoms;
   Y is O or S or NH or NCH₃;
   A is C₂₋₆ alkyl, optionally substituted with unsaturation and heteroatoms;
   B is individually selected from N(CH₃)₂,

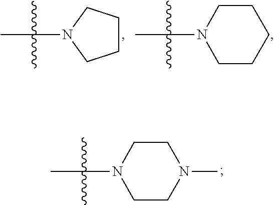

or not present;
D is N or C—H;
E is C or E is N when F is absent;
F is individually selected from H, OH, CN, methyl sulphone, 5-membered ring heterocycles including but limiting to N-triazolyl, C-triazolyl, thiazoles, oxadiazole, tetrazole, oxazoles, pyrazoles and thiadiazole rings; and
G is individually selected from H, OH, CN, methyl sulphone, 5-membered ring heterocycles including but limiting to N-triazolyl, C-triazolyl, thiazoles, oxadiazole, tetrazole, oxazoles, pyrazoles and thiadiazole rings.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched having about 1 to about 12 carbon atoms in the chain. Particular alkyl groups have 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. The term “alkylene” refers to a divalent group formed from an alkane by removal of two hydrogen atoms. Exemplary alkylene groups include, but are not limited to, methylene, ethylene, propylene, and other divalent groups derived from the alkanes described above.

The term “alkenyl” means an aliphatic hydrocarbon group containing a carbon-carbon double bond and which may be straight or branched having about 2 to about 6 carbon atoms in the chain. Particular alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “aryl” means an aromatic monocyclic or multicyclic ring system of 6 to about 14 carbon atoms, preferably of 6 to about 10 carbon atoms. Representative aryl groups include phenyl and naphthyl. The term “arylene” refers to a group obtained by removal of a hydrogen atom from an aryl group. Non-limiting examples of arylene include phenylene and naphthylene.

The term “heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like. The term “heteroarylene” refers to a group obtained by removal of a hydrogen atom from a heteroaryl group. Exemplary heteroarylene groups include, but are not limited to, groups derived from the heteroaryl groups described above.

As used herein, “heterocyclyl” refers to a stable 3- to 18-membered ring (radical) which consists of carbon atoms and from one to five heteroatoms selected from the group consisting of nitrogen, oxygen and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. For purposes of this application, when the heterocycle is a polycyclic ring system, one of the rings can be an aromatic ring and another one is non-aromatic. Examples of heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984).

The term “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., =O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

This technology also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

In the characterization of some of the substituents, it is recited that certain substituents may combine to form rings. Unless stated otherwise, it is intended that such rings may exhibit various degrees of unsaturation (from fully saturated to fully unsaturated), may include heteroatoms and may be substituted with lower alkyl or alkoxy.

The term “pharmaceutically acceptable salts” means the relatively non-toxic, inorganic, and organic acid addition salts, and base addition salts, of compounds of the present disclosure. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418). Base addition salts can also be prepared by separately reacting the purified compound in its acid form with a suitable organic or inorganic base and isolating the salt thus formed. Base addition salts include pharmaceutically acceptable metal and amine salts. Suitable metal salts include sodium, potassium, calcium, barium, zinc, magnesium, and aluminum salts. Sodium and potassium salts are preferred. Suitable inorganic base addition salts are prepared from metal bases which include, for example, sodium hydride, sodium hydroxide, potassium hydroxide, calcium hydroxide, aluminum hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylarnine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “solvate” refers to a compound of formula (I) in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “pharmaceutically acceptable” means it is, within the scope of sound medical judgement, suitable for use in contact with the cells of humans and lower animals without undue toxicity, irritation, allergic response and the like, and are commensurate with a reasonable benefit/risk ratio.

In some embodiments disclosed herein, the compound of formula (I) can be selected from the group consisting of

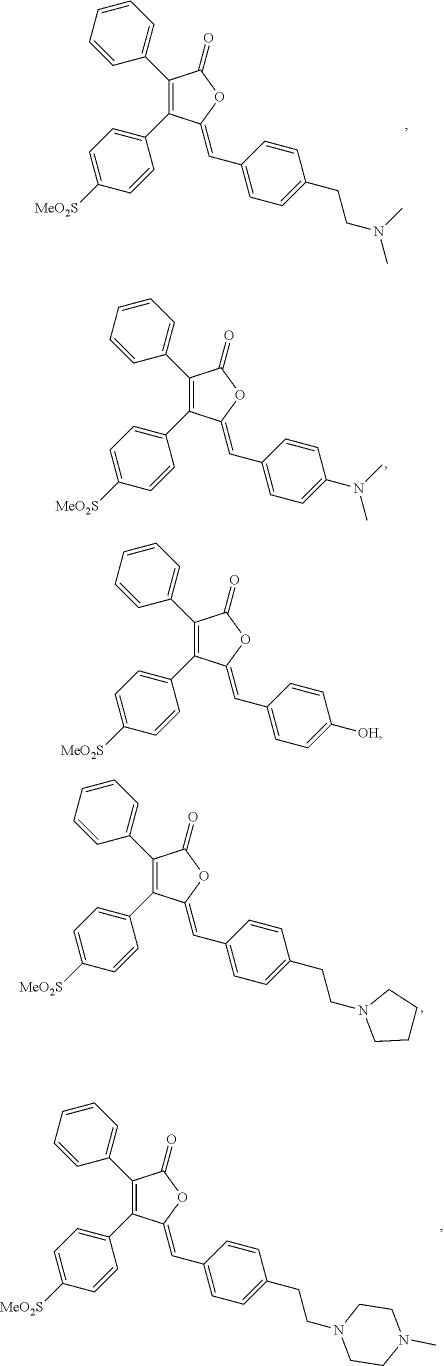

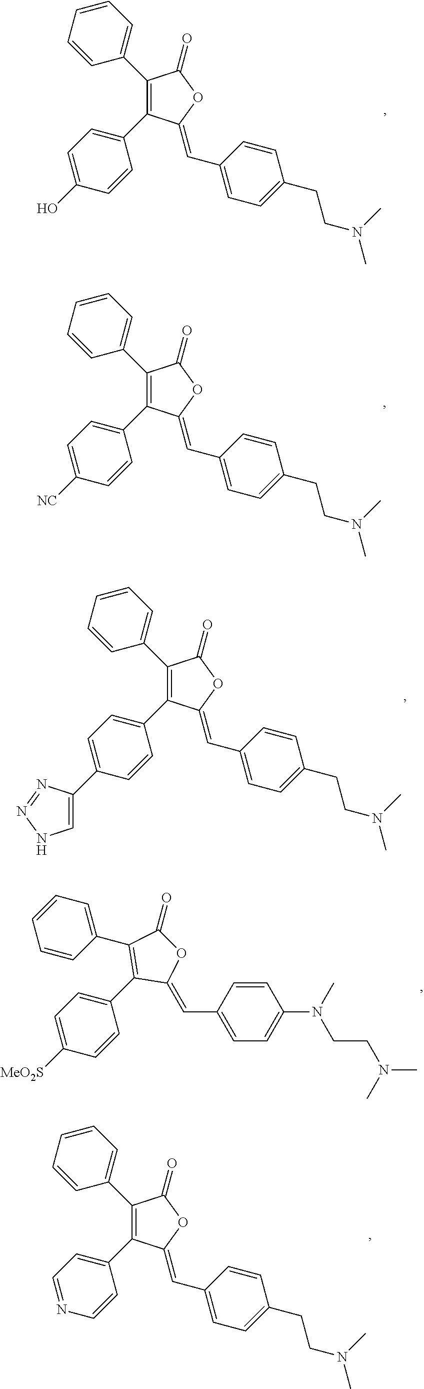 [SMILES: CN(C)CCc1ccc(/C=C2\OC(=O)C(c3ccccc3)=C2c2ccc(O)cc2)cc1 ; CN(C)CCc1ccc(/C=C2\OC(=O)C(c3ccccc3)=C2c2ccc(C#N)cc2)cc1 ; CN(C)CCc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCc1ccc(/C=C2\OC(=O)C(c3ccccc3)=C2c2ccncc2)cc1]

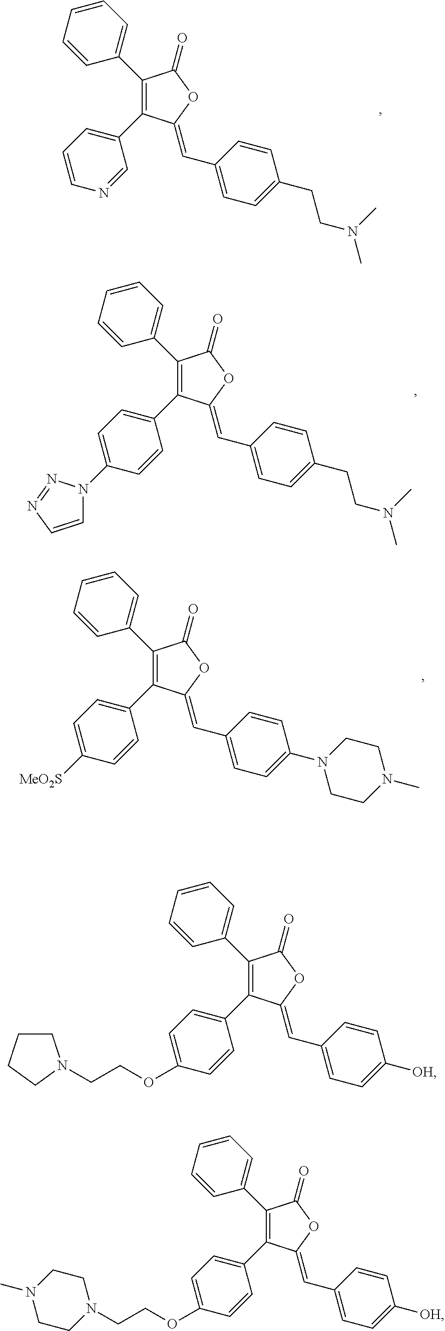

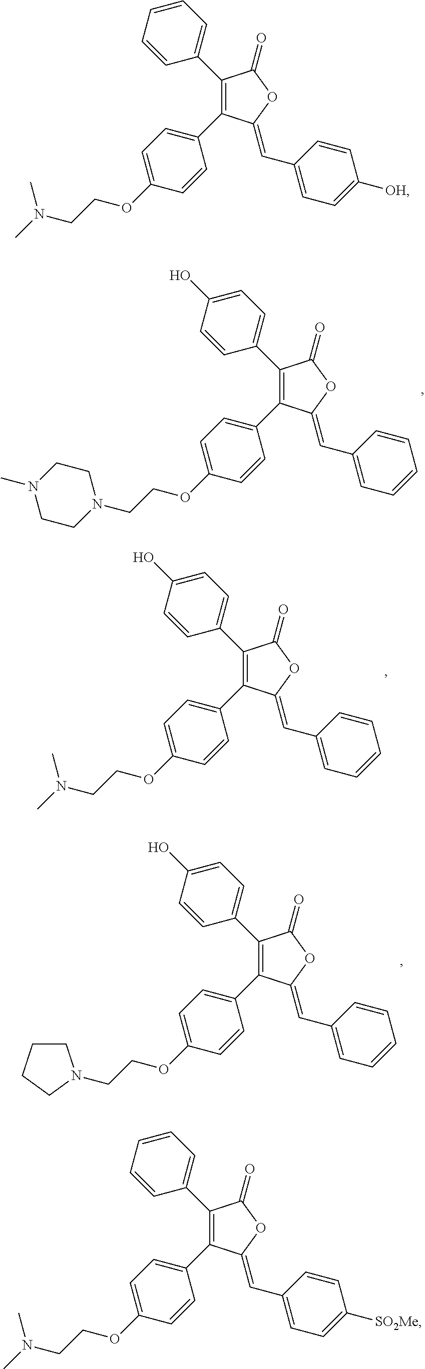 [SMILES: CN(C)CCOc1ccc(C2=C(c3ccccc3)C(=O)O/C2=C\c2ccc(O)cc2)cc1 ; CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1]

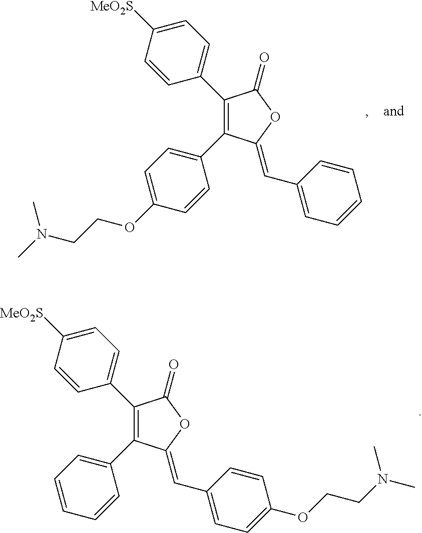 [SMILES: CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

In any of the embodiments disclosed herein, the compound of formula (I) can be selected from the group consisting of

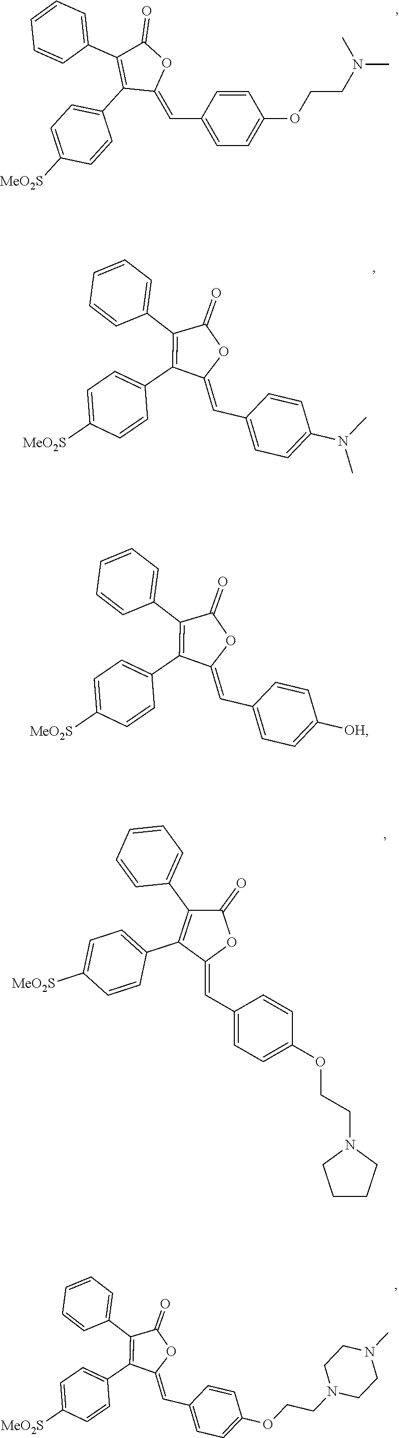 [SMILES: CN(C)CCOc1ccc(/C=C2\OC(=O)C(c3ccccc3)=C2c2ccc(S(C)(=O)=O)cc2)cc1 ; CN(C)c1ccc(/C=C2\OC(=O)C(c3ccccc3)=C2c2ccc(S(C)(=O)=O)cc2)cc1 ; CS(=O)(=O)c1ccc(C2=C(c3ccccc3)C(=O)O/C2=C\c2ccc(O)cc2)cc1 ; CS(=O)(=O)c1ccc(C2=C(c3ccccc3)C(=O)O/C2=C\c2ccc(OCCN3CCCC3)cc2)cc1 ; CN1CCN(CCOc2ccc(/C=C3\OC(=O)C(c4ccccc4)=C3c3ccc(S(C)(=O)=O)cc3)cc2)CC1]

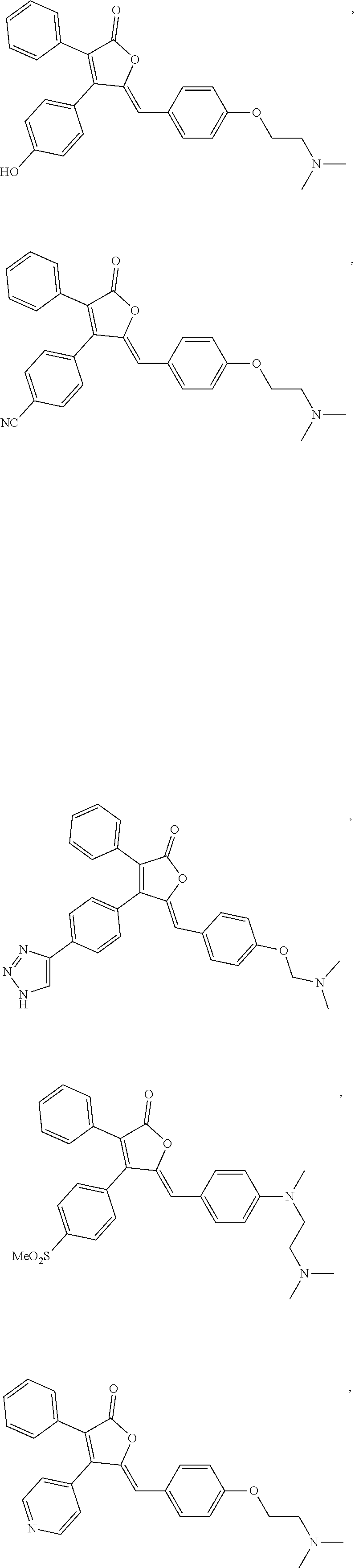 [SMILES: CN(C)CCOc1ccc(/C=C2\OC(=O)C(c3ccccc3)=C2c2ccc(O)cc2)cc1 ; CN(C)CCOc1ccc(/C=C2\OC(=O)C(c3ccccc3)=C2c2ccc(C#N)cc2)cc1 ; CN(C)COc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCOc1ccc(/C=C2\OC(=O)C(c3ccccc3)=C2c2ccncc2)cc1]

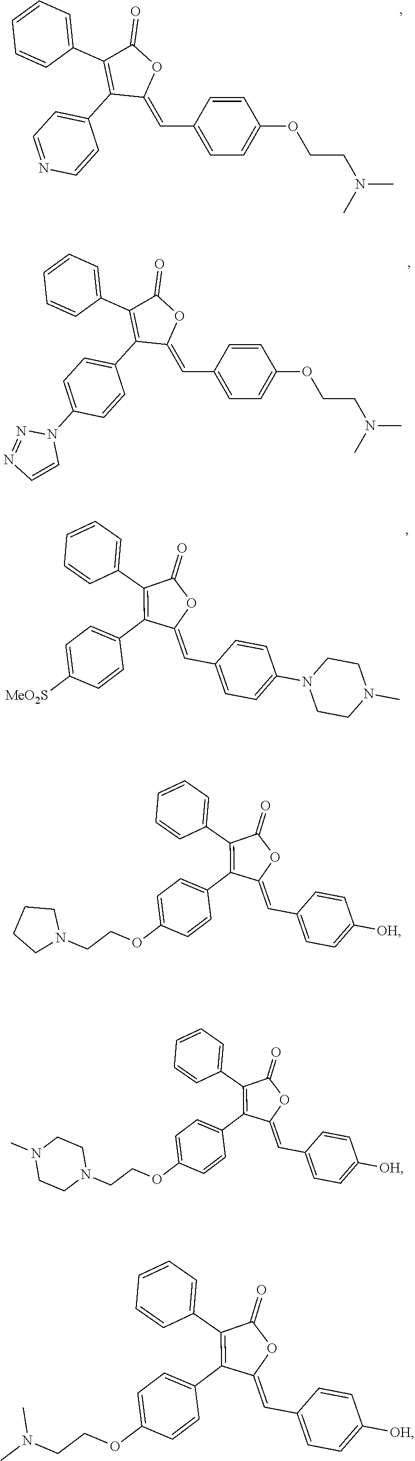

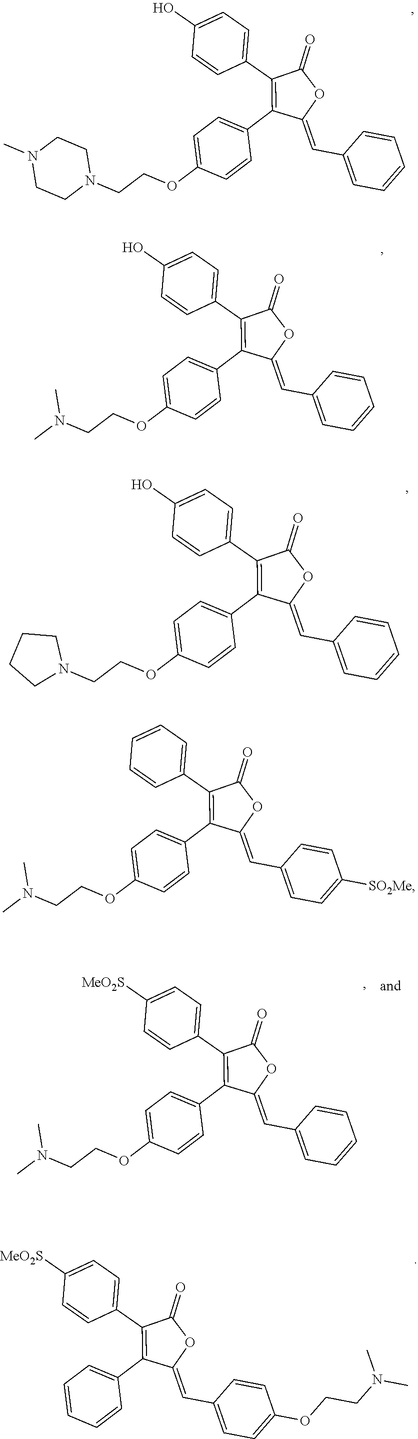 [SMILES: CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1 ; CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

Another exemplary embodiment of the present disclosure provides a method of treating cancer in a subject. The method can comprise providing a compound as disclosed herein to a subject in need of said treatment.

The term “method of treating” means amelioration or relief from the symptoms and/or effects associated with the disorders described herein. As used herein, reference to “treatment” of a patient is intended to include prophylaxis.

While it may be possible for compounds of formula (I), formula (II), and formula (III) to be administered as raw chemicals, it can be preferable to present them as a part of a pharmaceutical composition. Accordingly, another aspect of the present invention is a pharmaceutical composition containing a therapeutically effective amount of the compound of formula (I), formula (II), or formula (III), or a pharmaceutically acceptable salt thereof, and a pharmaceutically acceptable carrier.

The term “pharmaceutically acceptable dosage forms” means dosage forms of the compound of the invention, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition.

The compounds and pharmaceutical compositions of the present disclosure are particularly useful for the treatment of cancer. As used herein, the term “cancer” refers to a cellular disorder characterized by uncontrolled or dysregulated cell proliferation, decreased cellular differentiation, inappropriate ability to invade surrounding tissue, and/or ability to establish new growth at ectopic sites. The term “cancer” includes, but is not limited to, solid tumors and bloodborne tumors. The term “cancer” encompasses diseases of skin, tissues, organs, bone, cartilage, blood, and vessels. The term “cancer” further encompasses primary and metastatic cancers.

In any of the embodiments disclosed herein, the cancer can be an estrogen receptor (ER) dependent cancer (i.e., the cancer can depend on estrogen receptor (ER) signaling).

In any of the embodiments disclosed herein, the cancer can be selected from the group consisting of breast cancer, prostate cancer, lung cancer, and liver cancer.

In practicing the method of the present disclosure, agents suitable for treating a subject can be administered using any method standard in the art. The agents, in their appropriate delivery form, can be administered orally, intradermally, intramuscularly, intraperitoneally, intravenously, subcutaneously, or intranasally. The compositions of the present disclosure may be administered alone or with suitable pharmaceutical carriers, and can be in solid or liquid form, such as tablets, capsules, powders, solutions, suspensions, or emulsions.

Another exemplary embodiment of the present disclosure provides a method of inhibiting an estrogen receptor alpha. The method can comprise providing a compound as disclosed herein under conditions effective to inhibit the estrogen receptor alpha (ERa).

Another exemplary embodiment of the present disclosure provides a method of degrading an estrogen receptor alpha. The method can comprise providing a compound as disclosed herein under conditions effective to degrade the estrogen receptor alpha (ERα).

Another exemplary embodiment of the present disclosure provides a method of treating a hyperproliferative disorder. The method can comprise providing a compound as disclosed herein under conditions effective to treat a hyperproliferative disorder. In any of the embodiments disclosed herein, the hyperproliferative disorder can depend on ERα, RET, GREB1, ADCY5 or TFF1. In any of the embodiments disclosed herein, the hyperproliferative disorder can be a cancer selected from the group consisting of breast cancer, prostate cancer, lung cancer, and liver cancer.

Another exemplary embodiment of the present disclosure provides a composition comprising a compound as disclosed herein and a carrier.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

This written description uses examples to disclose certain example embodiments of the disclosed technology, including the best mode, and also to enable any person skilled in the art to practice certain embodiments of the disclosed technology, including making and using any devices or systems and performing any incorporated methods. The scope of certain embodiments of the disclosed technology is defined in the claims and their equivalents, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

An embodiment of the present disclosure may be implemented according to at least the following:
Clause 1: A compound of formula (I) having the following structure:

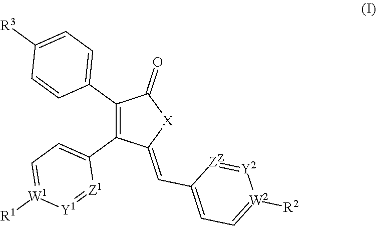

or a stereoisomer, pharmaceutically acceptable salt, oxide, or solvate thereof, wherein R¹ is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶; R² is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶; R³ is H, hydroxyl, —SO₂C₁₋₆ alkyl, —NR⁴R⁵, —CN, heterocyclyl, aryl, heteroaryl, —CH₂R⁶, —OR⁶ or —SR⁶; X is 0 or NR⁷; W¹ is C or N, wherein when W¹ is N, R¹ is not present; Y¹ is CH or N; Z¹ is CH or N; W² is C or N, wherein when W² is N, R² is not present; Y² is CH or N; Z² is CH or N; R⁴ is H, C₁-C₆ alkyl, C₂-C₆ alkenyl, wherein C₁-C₆ alkyl and C₂-C₆ alkenyl can be substituted with —N(CH₃)₂ or heterocyclyl; R⁵ is H or C₁-C₆ alkyl; or R⁴ and R⁵ combine with the nitrogen atom to which they are attached to form a heterocyclic ring, wherein the heterocyclic ring can be substituted with C₁-C₆ alkyl; R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.
Clause 2: The compound according to clause 1, selected from the group consisting of

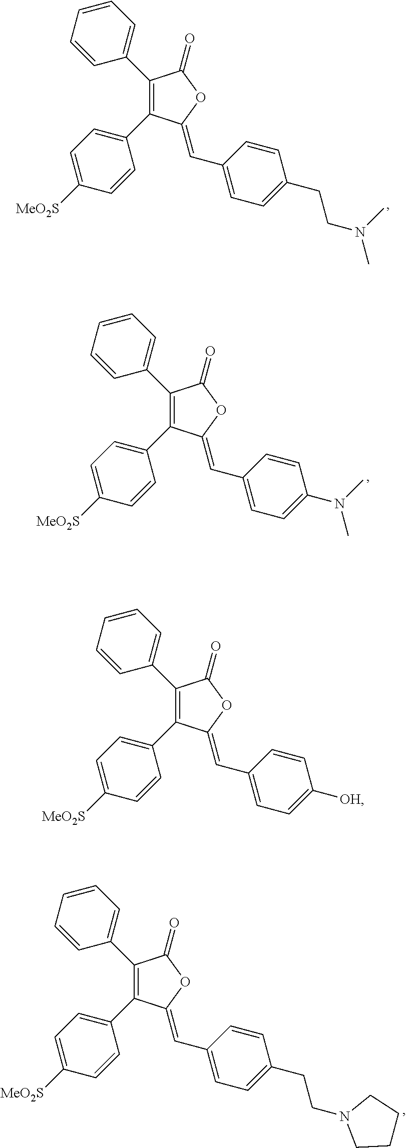

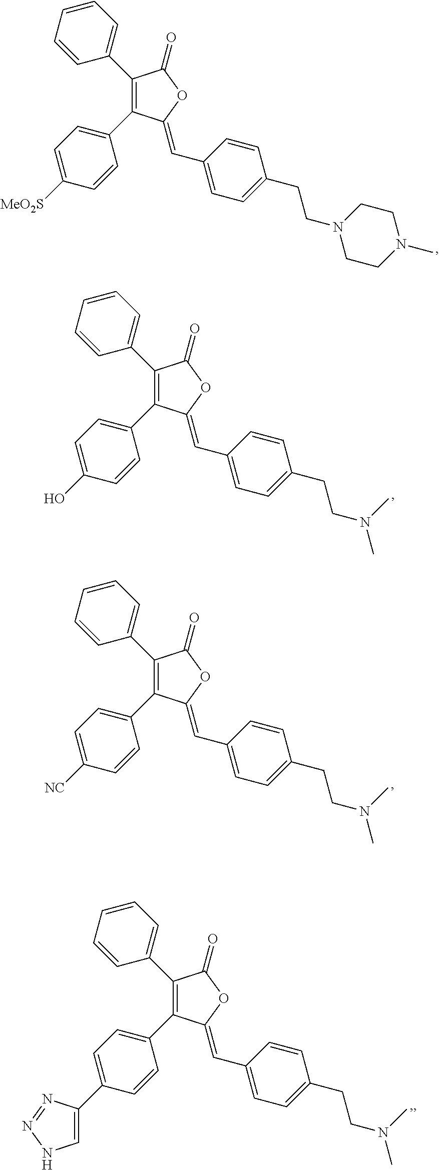

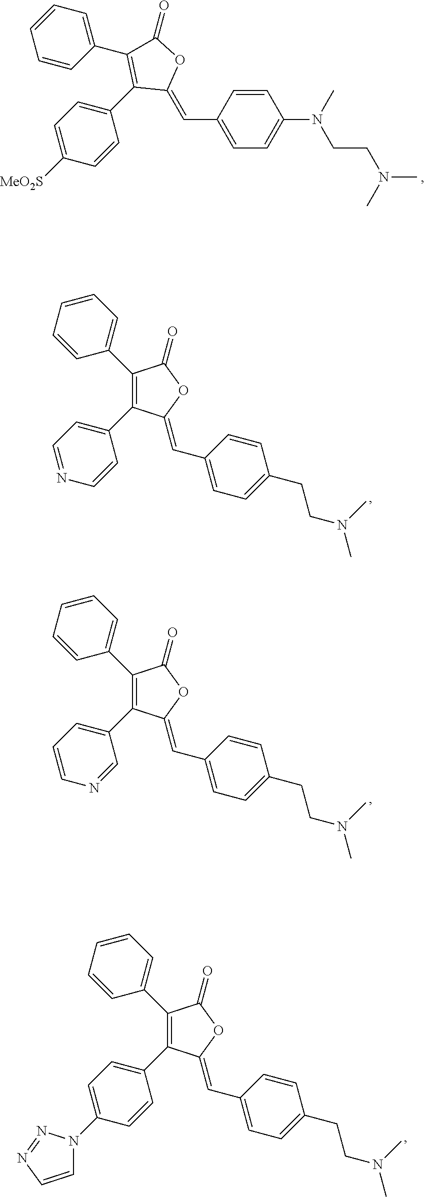

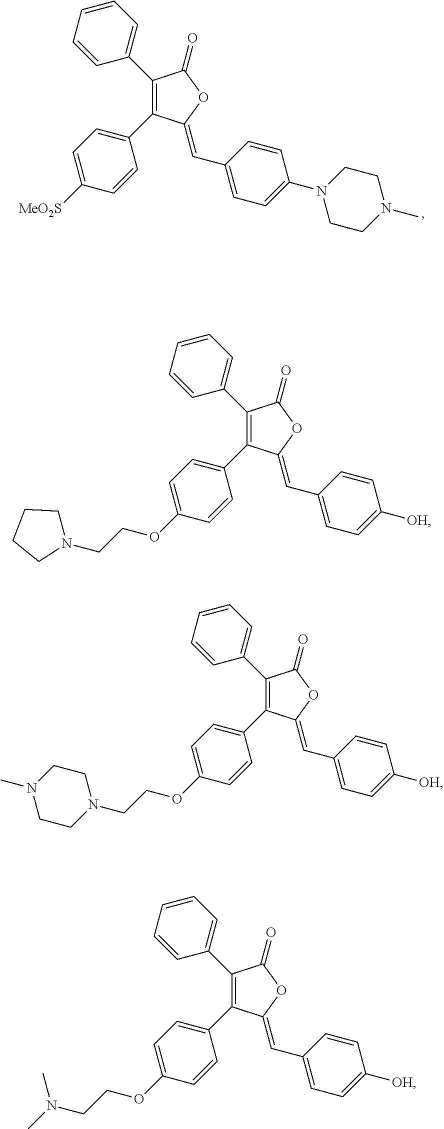

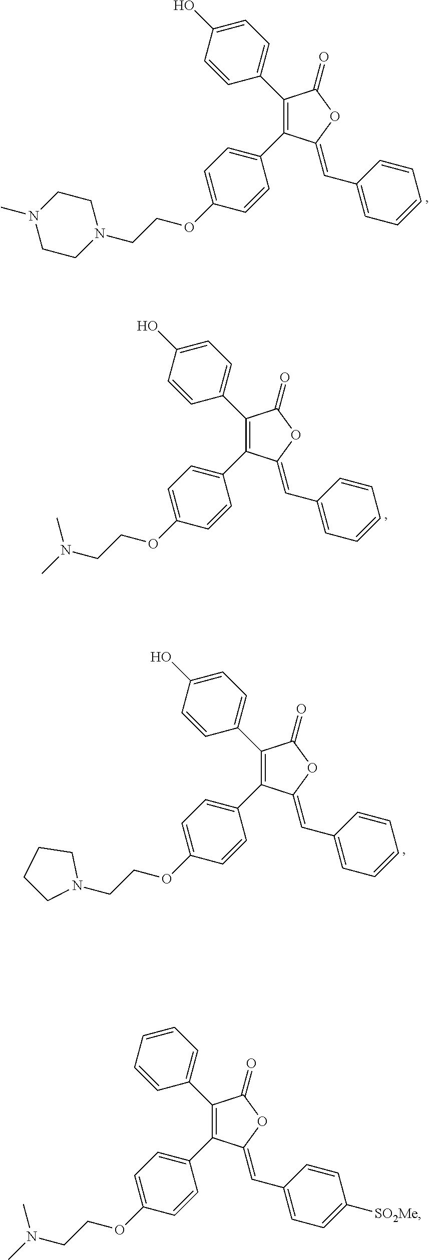

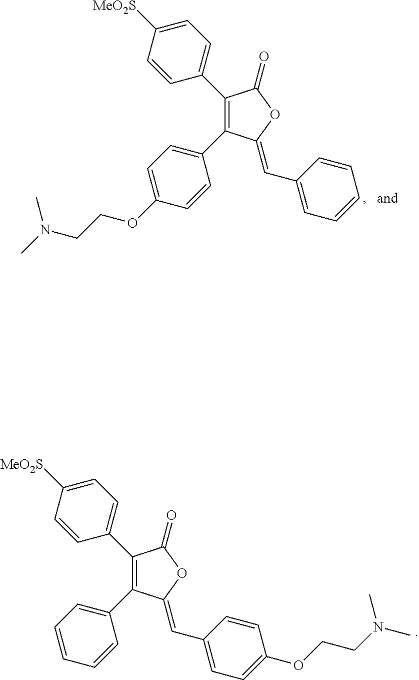 [SMILES: CN(C)CCOc1ccc(C2=C(c3ccc(S(C)(=O)=O)cc3)C(=O)O/C2=C\c2ccccc2)cc1 ; "," ; "and" ; CN(C)CCOc1ccc(/C=C2\OC(=O)C(c3ccc(S(C)(=O)=O)cc3)=C2c2ccccc2)cc1 ; "."]

Clause 3: A compound of formula (I) having the following structure:

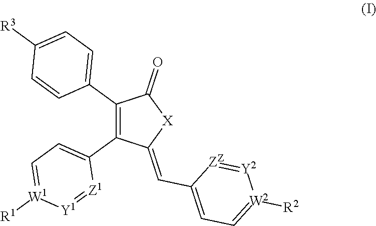

or a stereoisomer, pharmaceutically acceptable salt, oxide, or solvate thereof, wherein R¹ is optionally present, and when present is H, hydroxyl, —SO₂C₁₋₆ alkyl, —NR⁴R⁵, —CN, heterocyclyl, aryl, heteroaryl, —OR⁶ or —SR⁶; R² is optionally present, and when present is H, hydroxyl, —SO₂C₁₋₆ alkyl, —NR⁴R⁵, —CN, heterocyclyl, aryl, heteroaryl, —OR⁶ or —SR⁶; R³ is H, hydroxyl, —SO₂C₁₋₆ alkyl, —NR⁴R⁵, —CN, heterocyclyl, aryl, heteroaryl, —OR⁶ or —SR⁶; X is O or NR⁷; W¹ is C or N, wherein when W¹ is N, R¹ is not present; Y¹ is CH or N; Z¹ is CH or N; W² is C or N, wherein when W² is N, R² is not present; Y² is CH or N; Z² is CH or N; R⁴ is H, C₁-C₆ alkyl, C₂-C₆ alkenyl, wherein C₁-C₆ alkyl and C₂-C₆ alkenyl can be substituted with —N(CH₃)₂ or heterocyclyl; R⁵ is H or C₁-C₆ alkyl; or R⁴ and R⁵ combine with the nitrogen atom to which they are attached to form a heterocyclic ring, wherein the heterocyclic ring can be substituted with C₁-C₆ alkyl; R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.
Clause 4: The compound according to clause 3, selected from the group consisting of

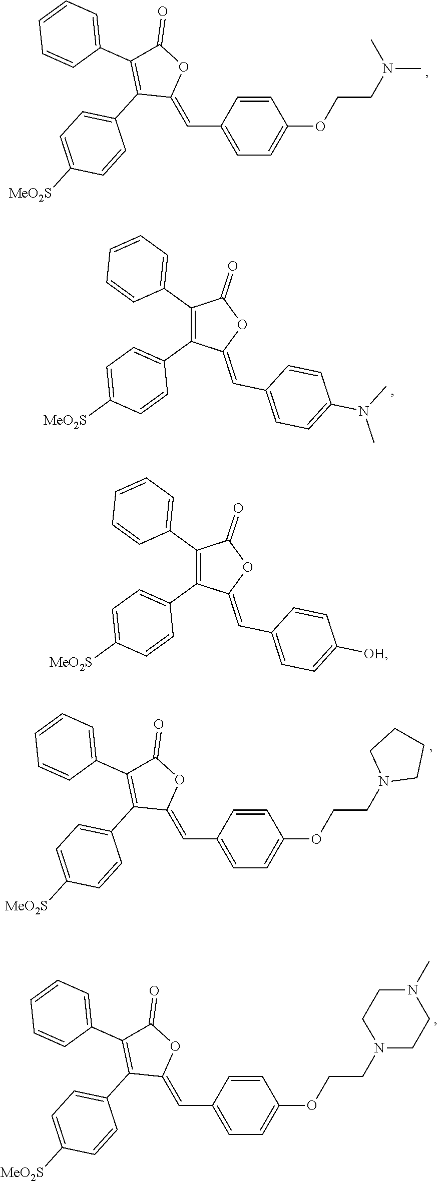 [SMILES: CN(C)CCOc1ccc(/C=C2\OC(=O)C(c3ccccc3)=C2c2ccc(S(C)(=O)=O)cc2)cc1 ; CN(C)c1ccc(/C=C2\OC(=O)C(c3ccccc3)=C2c2ccc(S(C)(=O)=O)cc2)cc1 ; CS(=O)(=O)c1ccc(C2=C(c3ccccc3)C(=O)O/C2=C\c2ccc(O)cc2)cc1 ; CS(=O)(=O)c1ccc(C2=C(c3ccccc3)C(=O)O/C2=C\c2ccc(OCCN3CCCC3)cc2)cc1 ; CN1CCN(CCOc2ccc(/C=C3\OC(=O)C(c4ccccc4)=C3c3ccc(S(C)(=O)=O)cc3)cc2)CC1]

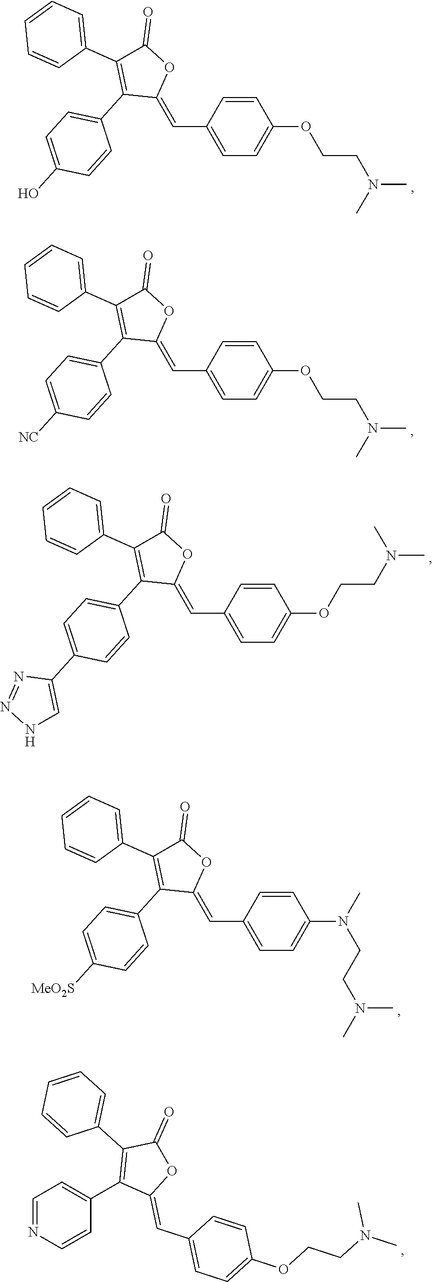 [SMILES: CN(C)CCOc1ccc(/C=C2\OC(=O)C(c3ccccc3)=C2c2ccc(O)cc2)cc1 ; CN(C)CCOc1ccc(/C=C2\OC(=O)C(c3ccccc3)=C2c2ccc(C#N)cc2)cc1 ; CN(C)CCOc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCOc1ccc(/C=C2\OC(=O)C(c3ccccc3)=C2c2ccncc2)cc1]

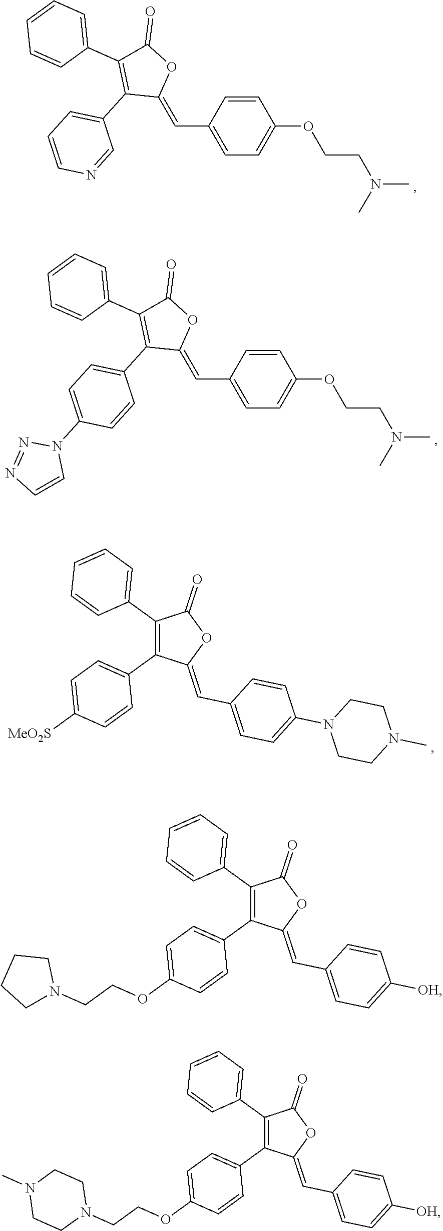

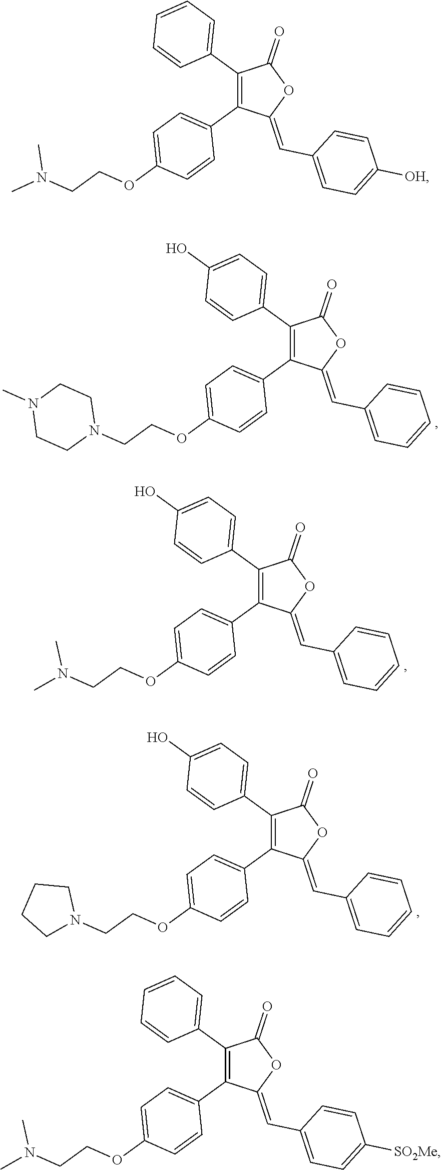 [SMILES: CN(C)CCOc1ccc(C2=C(c3ccccc3)C(=O)O/C2=C\c2ccc(O)cc2)cc1 ; CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1]

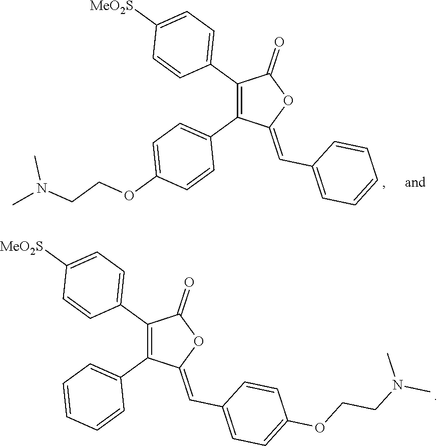 [SMILES: CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

Clause 5: A method of treating cancer in a subject, said method comprising: providing a compound according to clause 1 to a subject in need of said treatment.
Clause 6: The method of clause 5, wherein the cancer is an estrogen receptor (ER) dependent cancer.
Clause 7: The method of clause 6, wherein the cancer is selected from the group consisting of breast cancer, prostate cancer, lung cancer, and liver cancer.
Clause 8: A method of treating cancer in a subject, said method comprising: providing a compound according to clause 3 to a subject in need of said treatment.
Clause 9: The method of clause 8, wherein the cancer is an estrogen receptor (ER) dependent cancer.
Clause 10: The method of clause 9, wherein the cancer is selected from the group consisting of breast cancer, prostate cancer, lung cancer, and liver cancer.
Clause 11: A method of inhibiting an estrogen receptor alpha, said method comprising: providing a compound according to clause 1 under conditions effective to inhibit the estrogen receptor alpha (ERα).
Clause 12: A method of inhibiting an estrogen receptor alpha, said method comprising: providing a compound according to clause 3 under conditions effective to inhibit the estrogen receptor alpha (ERα).
Clause 13: A method of degrading an estrogen receptor alpha, said method comprising: providing a compound according to clause 1 under conditions effective to degrade the estrogen receptor alpha (ERα).
Clause 14: A method of degrading an estrogen receptor alpha, said method comprising: providing a compound according to clause 3 under conditions effective to degrade the estrogen receptor alpha (ERα).
Clause 15: A method of treating a hyperproliferative disorder, said method comprising: providing a compound according to clause 1 under conditions effective to treat a hyperproliferative disorder.
Clause 16: The method of clause 15, wherein the hyperproliferative disorder depends on ERα, RET, GREB1, ADCY5 or TFF1.
Clause 17: The method of clause 15, wherein the hyperproliferative disorder is a cancer selected from the group consisting of breast cancer, prostate cancer, lung cancer, and liver cancer.
Clause 18: A method of treating a hyperproliferative disorder, said method comprising: providing a compound according to clause 3 under conditions effective to treat a hyperproliferative disorder.
Clause 19: A composition comprising: a compound according to clause 1; and a carrier.
Clause 20: A composition comprising: a compound according to clause 3; and a carrier.

### EXAMPLES

The following Examples are presented to illustrate various aspects of the present disclosure but are by no means intended to limit its scope.

### Example 1—Synthesis of Exemplifying Compounds

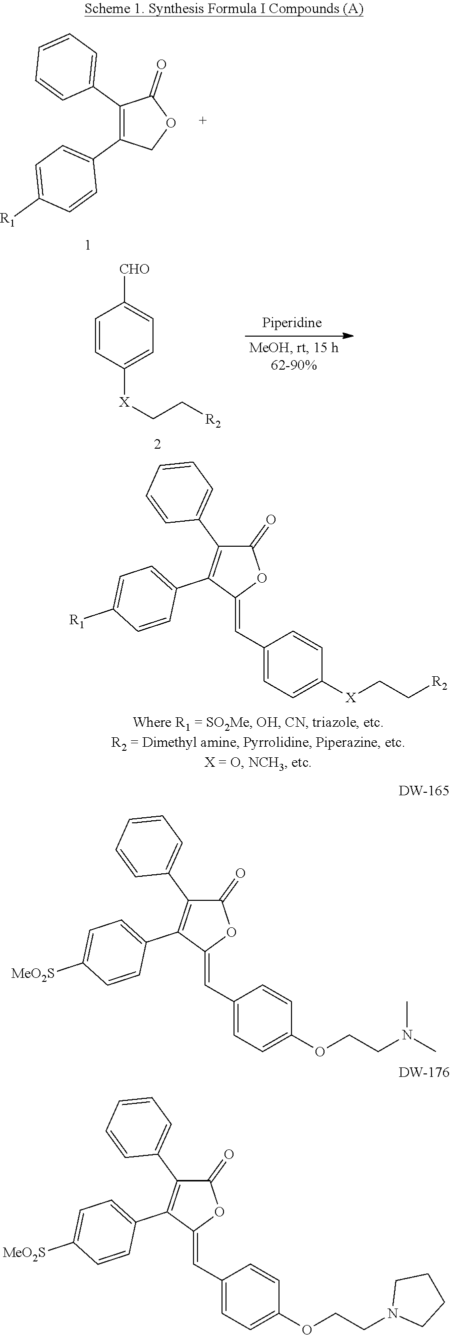

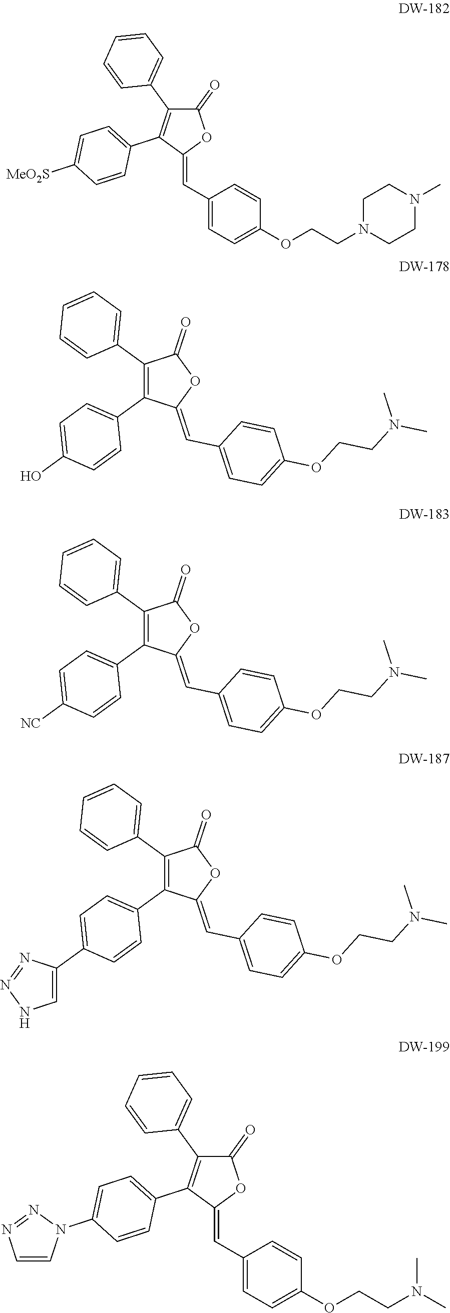 [SMILES: CN1CCN(CCOc2ccc(/C=C3\OC(=O)C(c4ccccc4)=C3c3ccc(S(C)(=O)=O)cc3)cc2)CC1 ; CN(C)CCOc1ccc(/C=C2\OC(=O)C(c3ccccc3)=C2c2ccc(O)cc2)cc1 ; CN(C)CCOc1ccc(/C=C2\OC(=O)C(c3ccccc3)=C2c2ccc(C#N)cc2)cc1 ; CN(C)CCOc1ccc(/C=C2\OC(=O)C(c3ccccc3)=C2c2ccc(-c3c[nH]nn3)cc2)cc1 ; CN(C)CCOc1ccc(/C=C2\OC(=O)C(c3ccccc3)=C2c2ccc(-n3ccnn3)cc2)cc1]

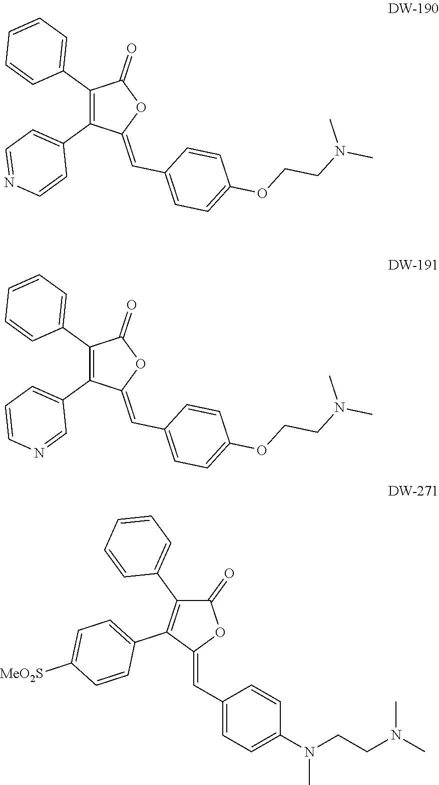 [SMILES: CN(C)CCOc1ccc(/C=C2\OC(=O)C(c3ccccc3)=C2c2ccncc2)cc1 ; CN(C)CCOc1ccc(/C=C2\OC(=O)C(c3ccccc3)=C2c2cccnc2)cc1 ; CN(C)CCN(C)c1ccc(/C=C2\OC(=O)C(c3ccccc3)=C2c2ccc(S(C)(=O)=O)cc2)cc1]

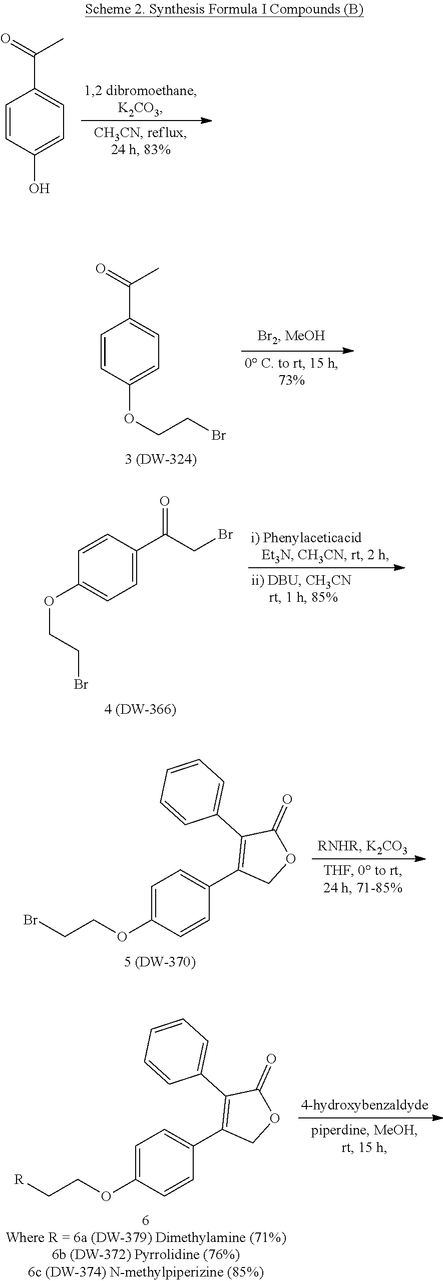

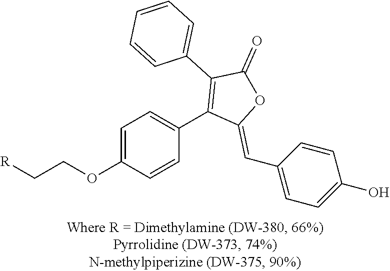

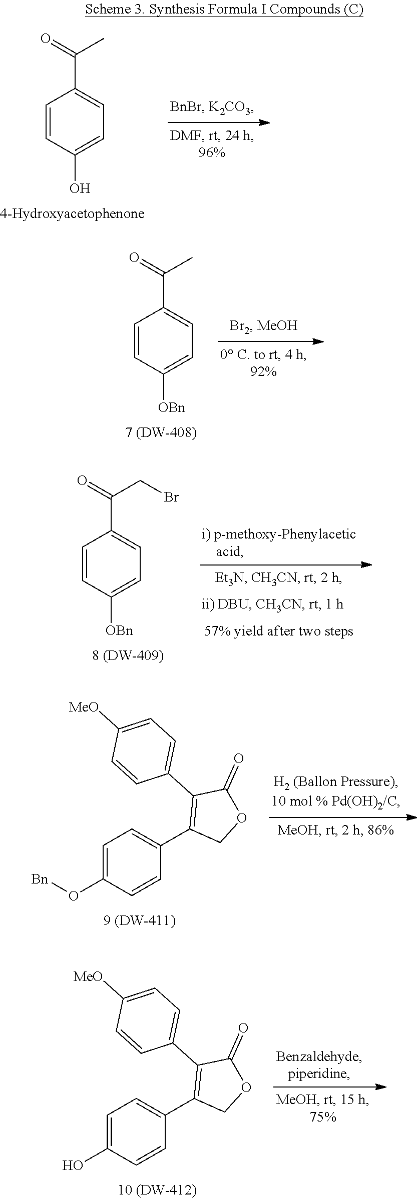

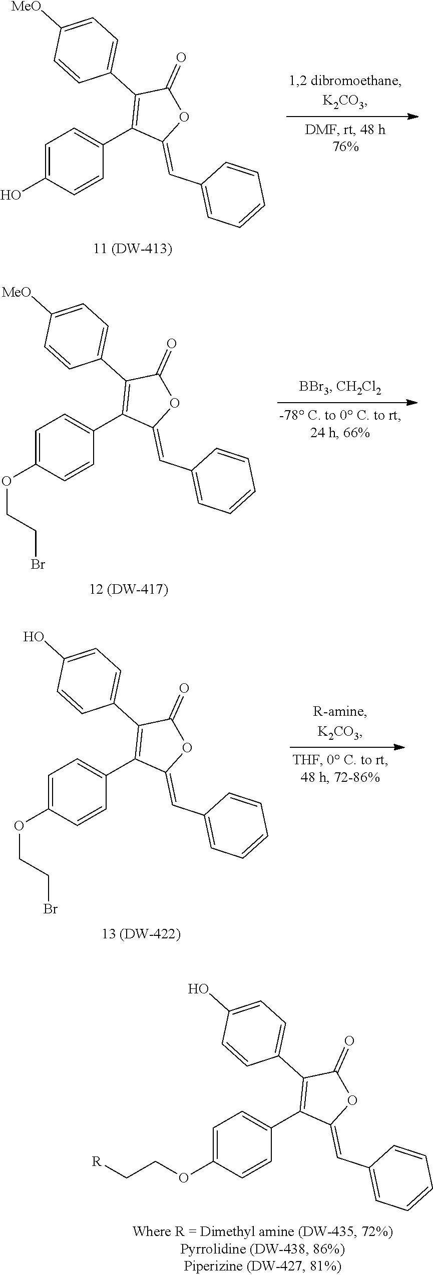

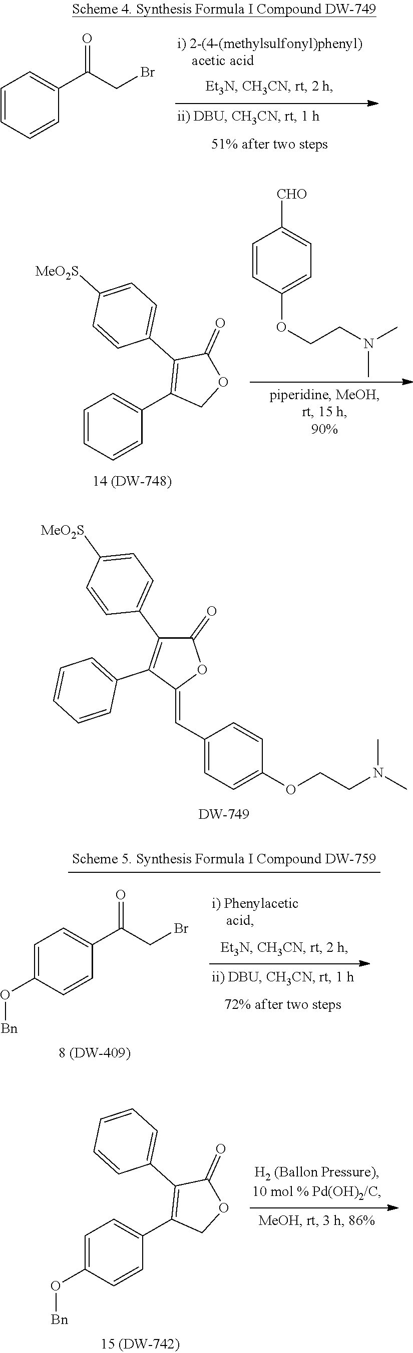 [SMILES: O=C(CBr)c1ccccc1 ; CS(=O)(=O)c1ccc(C2=C(c3ccccc3)COC2=O)cc1 ; CN(C)CCOc1ccc(C=O)cc1 ; CN(C)CCOc1ccc(/C=C2\OC(=O)C(c3ccc(S(C)(=O)=O)cc3)=C2c2ccccc2)cc1 ; O=C(CBr)c1ccc(OCc2ccccc2)cc1 ; O=C1OCC(c2ccc(OCc3ccccc3)cc2)=C1c1ccccc1]

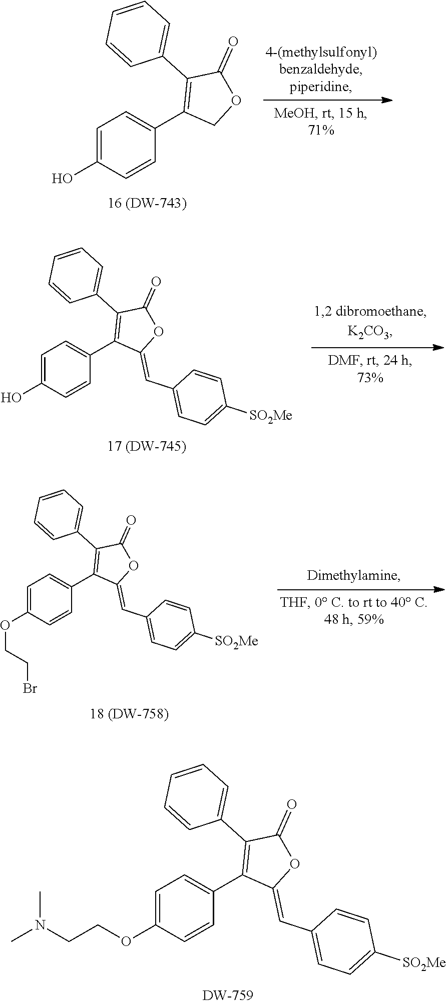 [SMILES: O=C1OCC(c2ccc(O)cc2)=C1c1ccccc1 ; CS(=O)(=O)c1ccc(/C=C2\OC(=O)C(c3ccccc3)=C2c2ccc(O)cc2)cc1 ; CS(=O)(=O)c1ccc(/C=C2\OC(=O)C(c3ccccc3)=C2c2ccc(OCCBr)cc2)cc1 ; CN(C)CCOc1ccc(C2=C(c3ccccc3)C(=O)O/C2=C\c2ccc(S(C)(=O)=O)cc2)cc1]

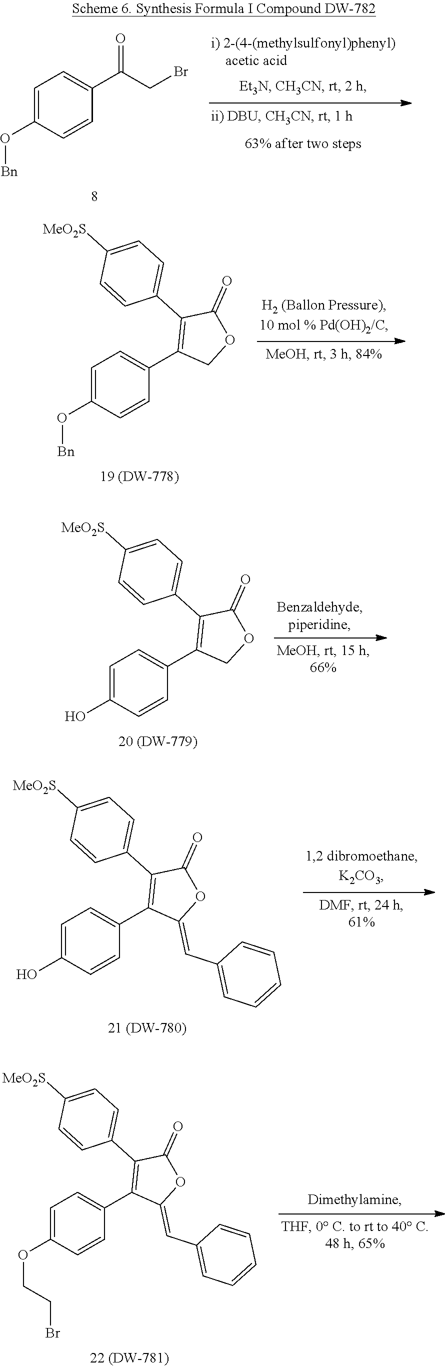 [SMILES: O=C(CBr)c1ccc(OCc2ccccc2)cc1 ; CS(=O)(=O)c1ccc(C2=C(c3ccc(OCc4ccccc4)cc3)COC2=O)cc1 ; CS(=O)(=O)c1ccc(C2=C(c3ccc(O)cc3)COC2=O)cc1 ; CS(=O)(=O)c1ccc(C2=C(c3ccc(O)cc3)/C(=C/c3ccccc3)OC2=O)cc1 ; CS(=O)(=O)c1ccc(C2=C(c3ccc(OCCBr)cc3)/C(=C/c3ccccc3)OC2=O)cc1]

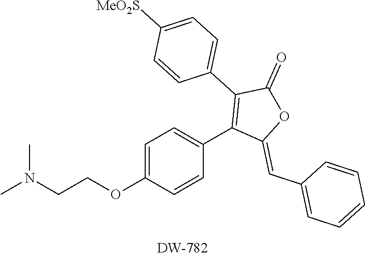 [SMILES: CN(C)CCOc1ccc(C2=C(c3ccc(S(C)(=O)=O)cc3)C(=O)O/C2=C\c2ccccc2)cc1]

### Example 2—Experimental Procedures and Characterizations

### Experimental Procedure & Characterization of DW-165 Family

### General Procedure for the Synthesis of DW-165 to DW-271

A mixture of Piperidine (1.1 equiv.), was added to a mixture of furanone (1) (1 equiv.) and aromatic aldehyde (2) (2 equiv.) were dissolved in 5 mL of methanol and the reaction was stirred at rt for 15 h in a dark atmosphere. The completion of the reaction was indicated by TLC. The reaction was quenched with water (50 mL), extracted with CH₂Cl₂ (2×20 mL), and the combined organic layer was sequentially washed with a saturated solution of brine (30 mL). The organic layer was dried over Na₂SO₄, and then filtered. The solvent was removed using a rotary evaporator and the crude material was purified on silica gel column chromatography, eluting with CH₂Cl₂:Methanol (10:1), v/v, to afford solid the desired product in 66-91% yield.
DW-165: Following the general procedure using 4-(4-(methylsulfonyl)phenyl)-3-phenylfuran-2(5H)-one (1) (100 mg), 4-(2-(dimethylamino)ethoxy)benzaldehyde(2) (122 mg) and Piperidine (0.030 ml) gave DW-165 (140 mg, 91% yield) as a yellow solid; ¹H NMR (400 MHz, CDCl₃) δ 8.05 (d, J=8.1 Hz, 2H), 7.74 (d, J=6.8 Hz, 2H), 7.56 (d, J=8.0 Hz, 2H), 7.37-7.27 (m, 5H), 6.95 ((d, J=9.0 Hz, 2H), 5.84 (s, 1H), 4.10 (t, J=5.7 Hz, 2H), 3.16 (s, 3H), 2.74 (t, J=5.7 Hz, 2H), 2.34 (s, 6H). ¹³C NMR (101 MHz, CDCl₃) δ 168.3, 149.3, 148.8, 144.1, 141.9, 136.1, 135.3, 133.1, 130.8, 129.9, 129.4, 128.9, 128.0, 122.4, 120.5, 115.7, 112.5, 65.2, 58.0, 47.7, 45.4, 43.8.
DW-176: Following the general procedure using 4-(4-(methylsulfonyl)phenyl)-3-phenylfuran-2(5H)-one (1) (100 mg), 4-(2-(pyrrolidin-1-yl)ethoxy)benzaldehyde (2) (139 mg) and Piperidine (0.030 ml) gave DW-176 (142 mg, 86% yield) as a yellow solid; ¹H NMR (400 MHz, CDCl₃) δ 8.04 (d, J=8.0 Hz, 2H), 7.73 (d, J=8.8 Hz, 2H), 7.55 (d, J=8.1 Hz, 2H), 7.37-7.23 (m, 5H), 6.92 (d, J=8.7 Hz, 2H), 5.83 (s, 1H), 4.13 (t, J=6.0 Hz, 2H), 3.14 (s, 3H), 2.91 (t, J=6.0 Hz, 2H), 2.64 (s, 4H), 1.80 (s, 4H).)¹³C NMR (101 MHz, CDCl3) δ 168.4, 150.3, 149.5, 146.5, 142.2, 136.0, 131.6, 130.7, 130.6, 129.4, 129.4, 129.0, 128.0, 124.9, 123.0, 120.6, 116.8, 112.3, 65.2, 58.0, 52.6, 43.7, 24.9.
DW-178: Following the general procedure using 4-(4-hydroxyphenyl)-3-phenylfuran-2(5H)-one (1) (100 mg), 4-(2-(dimethylamino)ethoxy)benzaldehyde (2) (152 mg) and Piperidine (0.039 ml) gave DW-178 (133 mg, 78% yield) as a yellow solid; ¹H NMR (400 MHz, DMSO) δ 7.75 (d, J=9.1 Hz, 2H), 7.30 (s, 5H), 7.16 (d, J=8.6 Hz, 2H), 7.00 (d, J=9.1 Hz, 2H), 6.84 (d, J=8.7 Hz, 2H), 6.08 (s, 1H), 5.73 (s, 1H), 4.07 (t, J=5.7 Hz, 2H), 2.59 (t, J=5.8 Hz, 2H), 2.47 (s, 3H), 2.18 (s, 6H). ¹³C NMR (101 MHz, CDCl₃) δ 169.5, 159.4, 150.7, 146.5, 132.6, 130.6, 129.7, 129.1, 128.2, 122.7, 115.3, 114.8, 65.2, 58.1, 45.4.
DW-182: Following the general procedure using 4-(4-(methylsulfonyl)phenyl)-3-phenylfuran-2(5H)-one (1) (100 mg), 4-(2-(4-methylpiperazin-1-yl)ethoxy)benzaldehyde (2) (157 mg) and Piperidine (0.031 ml) gave DW-182 (148 mg, 85% yield) as a yellow solid; ¹H NMR (400 MHz, CDCl₃) δ 8.59 (d, J=8.6 Hz, 2H), 8.28 (d, J=7.3 Hz, 2H), 8.11 (d, J=8.6 Hz, 2H), 7.91-7.64 (m, 5H), 7.46 (d, J=9.0 Hz, 2H), 6.40 (s, 1H), 4.14 (t, 2H), 2.85 (t, J=7.1, 4.9 Hz, 2H), 2.56 (s, 4H), 2.47 (s, 3H), 2.31 (s, 3H). ¹³C NMR (101 MHz, CDCl₃) δ 168.2, 151.7, 149.4, 145.2, 142.0, 135.9, 132.7, 130.8, 129.7, 129.4, 129.1, 128.9, 128.0, 123.6, 123.2, 123.0, 114.8, 114.6, 65.4, 56.8, 54.8, 47.1, 46.2, 43.8.
DW-183: Following the general procedure using 4-(5-oxo-4-phenyl-2,5-dihydrofuran-3-yl)benzonitrile (1) (100 mg), 4-(2-(dimethylamino)ethoxy)benzaldehyde(2) (2) (147 mg) and Piperidine (0.041 ml) gave DW-183 (148 mg, 81% yield) as a Yellow solid; ¹H NMR (400 MHz, CDCl₃) δ 7.72 (d, J=8.7 Hz, 2H), 7.56 (d, J=8.8 Hz, 2H), 7.37 (s, 2H), 7.28-7.15 (m, 4H), 6.91 (d, J=8.9 Hz, 2H), 5.90 (s, 1H), 4.09 (t, J=5.7 Hz, 2H), 2.74 (t J=5.7 Hz, 2H), 2.34 (s, 6H)¹³C NMR (101 MHz, CDCl₃) δ 169.5, 159.4, 150.7, 146.5, 132.6, 130.6, 129.7, 129.1, 128.2, 122.7, 119.0 116.3, 114.8, 65.2, 58.1, 45.4.
DW-187: Following the general procedure using 4-(4-(1H-1,2,3-triazol-4-yl)phenyl)-3-phenylfuran-2(5H)-one (1) (100 mg), 4-(2-(dimethylamino)ethoxy)benzaldehyde (2) (126 mg) and Piperidine (0.036 ml) gave DW-187 (105 mg, 66% yield) as a yellow solid; ¹H NMR (400 MHz, DMSO-d6) 7.86 (d, J=9.1 Hz, 2H), 7.65 (s, 1H), 7.34 (s, 2H), 7.2 (m, 5H), 7.16 (d, J=8.6 Hz, 2H), 6.90 (d, J=9.1 Hz, 2H), 6.08 (s, 1H), 5.73 (s, 1H), 3.99 (t, J=5.7 Hz, 2H), 3.2 (t, J=5.8 Hz, 2H), 2.47 (s, 3H), 2.18 (s, 6H); ¹³C NMR (101 MHz, CDCl₃) δ 169.5, 159.4, 150.7, 146.5, 132.6, 130.6, 129.7, 129.1, 128.2, 122.7, 115.3, 114.8, 65.2, 58.1, 45.4.
DW-190: Following the general procedure using 3-phenyl-4-(pyridin-4-yl)furan-2(5H)-one (1) (100 mg), 4-(2-(dimethylamino)ethoxy)benzaldehyde (2) (162 mg) and Piperidine (0.045 ml) gave DW-190 (126 mg, 73% yield) as a Yellow solid; ¹H NMR (400 MHz, CDCl₃) δ 8.76 (d, J=8.7 Hz 2H), 7.76 (d, J=8.4 Hz, 2H), 7.38 (s, 2H), 7.32-7.16 (m, 5H), 6.96 (d, J=8.8 Hz 2H), 5.87 (s, 1H), 4.11 (t, 2H), 2.77 (t, 2H), 2.36 (s, 6H). ¹³C NMR (101 MHz, CDCl₃) δ 169.7, 159.6, 150.9, 146.5, 142, 132.6, 130.6, 129.7, 129.1, 128.2, 122.7, 115.3, 114.8, 65.2, 58.1, 45.4.
DW-191: Following the general procedure using 3-phenyl-4-(pyridin-3-yl)furan-2(5H)-one (1) (100 mg), 4-(2-(dimethylamino)ethoxy)benzaldehyde (2) (162 mg) and Piperidine (0.045 ml) gave DW-190 (132 mg, 76% yield) as a yellow solid; ¹H NMR (400 MHz, CDCl₃) δ 7.77 (d, 4H), 7.50 (s, 2H), 7.37-7.29 (s, 4H), 6.95 (d, 2H), 5.89 (s, 1H), 4.16 (s, 2H), 2.87 (s, 2H), 2.42 (s, 6H)¹³C NMR (101 MHz, CDCl₃) δ 169.7, 159.6, 150.9, 146.5, 142, 132.6, 130.6, 129.7, 129.1, 128.2, 122.7, 115.3, 114.8, 65.2, 58.1, 45.4.
DW-199: Following the general procedure using 4-(4-(1H-1,2,3-triazol-1-yl)phenyl)-3-phenylfuran-2(5H)-one (1) (100 mg), 4-(2-(dimethylamino)ethoxy)benzaldehyde (2) (126 mg) and Piperidine (0.035 ml) gave DW-199 (120 mg, 76% yield) as a yellow solid; ¹H NMR (400 MHz, CDCl₃) δ 8.02 (d, J=8 Hz, 1H), 7.86 (d, J=8.1 Hz, 1H), 7.75 (d, J=8.7 Hz, 2H), 7.94-7.49 (d, J=8.8 Hz, 2H), 7.41 (m, 3H), 7.27 (m, 4H), 6.93 (d, J=8.8 Hz, 2H), 5.94 (s, 1H), 4.14 (t, 2H), 2.81 (s, 2H), 2.40 (s, 6H). ¹³C NMR (101 MHz, CDCl₃) δ 169.5, 159.4, 150.7, 146.5, 134.3 132.6, 130.6, 129.7, 129.1, 128.2, 122.7, 120.1, 116.3, 114.8, 65.2, 58.1, 45.4.
DW-271: Following the general procedure using 4-(4-(methylsulfonyl)phenyl)-3-phenylfuran-2(5H)-one (1) (90 mg), 4-((2-(dimethylamino)ethyl)(methyl)amino)benzaldehyde (118 mg) and Piperidine (0.031 ml) gave DW-271 (120 mg, 84% yield) as a red solid; ¹H NMR (400 MHz, CDCl₃) δ 8.04 (d, J=7.8 Hz, 2H), 7.70 (d, J=9.2 Hz, 2H), 7.57 (d, J=7.9 Hz, 2H), 7.34 (s, 2H), 7.30-7.25 (m, 3H), 6.68 (d, J=9.2 Hz, 2H), 5.84 (s, 1H), 3.52 (t, J=7.5 Hz, 2H), 3.16 (s, 3H), 3.05 (s, 3H), 2.50 (t, J=7.3 Hz, 2H), 2.31 (S, 6H).)¹³C NMR (101 MHz, CDCl₃) δ 168.2, 151.7, 149.4, 145.2, 142.0, 135.9, 132.7, 130.8, 129.7, 129.4, 129.1, 128.9, 128.0, 123.6, 123.2, 123.0, 114.8, 114.6, 54.8, 47.1, 46.2, 43.8.

### Synthesis of DW-Flip-1 Based on DW-178

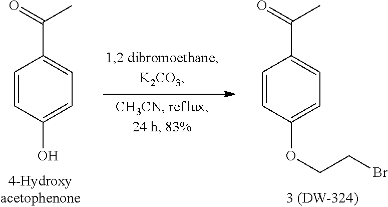

A solution of 4-hydroxy-acetopheone (1 g, 1 equiv.) in dry acetonitrile (30 mL) was stirred under an argon atmosphere and cooled to 0° C. K₂CO₃ (2.02 g, 2 equiv.) was added and the mixture was allowed to warm up to rt and kept stirring at rt for 30 min. Subsequently, 1, 2-dibromoethane (1.9 1 mL, 3 equiv.) was added dropwise to the mixture, and the solution was reflux for 24 h. After completion of the reaction as indicated by TLC, the reaction was quenched with water (50 mL) and extracted with CH₂Cl₂ (3×20 mL). The combined organic layer was dried over Na₂SO₄ and then filtered. The solvent was removed using a rotary evaporator, and the crude was purified by silica gel chromatography, eluting with Hexane:ethylacetate (9:1), v/v, to furnish the desired 1-(4-(2-bromoethoxy)phenyl)ethan-1-one. yield 3 (DW-324): 1.5 g g (83%). ¹H NMR (400 MHz, CDCl₃) δ 7.97 (d, J=9.8 Hz, 2H), 6.97 (d, J=6.8 Hz, 2H), 4.37 (t, 2H), 3.66 (t, 2H), 2.34 (s, 3H).

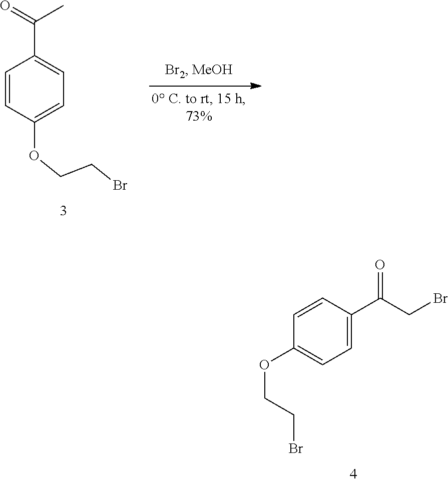

Bromine (0.208 mL, 1 equiv.) was added drop wise to a solution of 1-(4-(2-bromoethoxy)phenyl)ethan-1-one (1 g, lequiv.) in methanol (20 mL) was added at 0° C. and the mixture was allowed to warm up to rt and kept stirring at rt 15 h. After completion of the reaction as indicated by TLC, the reaction was quenched with water (50 mL) and Precipitate was filter and residue yielded a pure product compound 4(DW-366) as white solid in 950 mg (73%). ¹H NMR (400 MHz, CDCl₃) δ 7.97 (d, J=9.8 Hz, 2H), 6.97 (d, J=6.8 Hz, 2H), 4.41-4.39 (s, 2H), 4.37 (t, 2H), 3.66 (t, 2H).

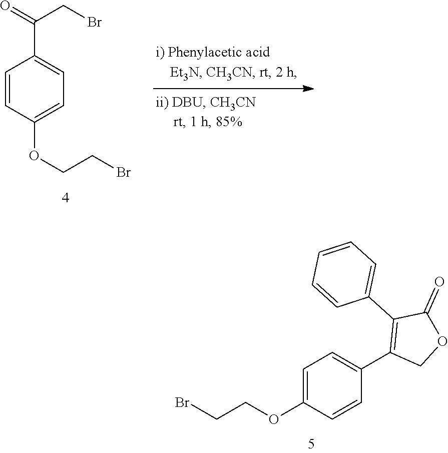 [SMILES: O=C(CBr)c1ccc(OCCBr)cc1 ; O=C1OCC(c2ccc(OCCBr)cc2)=C1c1ccccc1]

To a stirred solution of substitutedphenacyl bromide (4) (0.8 g 1 equiv.) and phenylacetic acid (0.340 g, 1 equiv.) in acetonitrile (10 ml) was added triethylamine (0.416 ml, 1 equiv.). The resultant mixture was stirred for 2 h at rt. The completion of the reaction as indicated by TLC. 1,8 Diazabicyclo[5.4.0]undec-7-ene (DBU) (0.362 g, 1 equiv.) was added and stirred for 1 h. Water (30 mL) was added and extracted with ethyl acetate (3×20 ml). The combined organic layer was dried over Na2SO4 and then filtered. The solvent was removed using a rotary evaporator, and the crude was purified by silica gel chromatography, eluting with Hexane:ethylacetate (4:1), v/v, to furnish the desired 4-(4-(2-bromoethoxy) phenyl)-3-phenylfuran-2(5H)-one 5 (DW-370) as solid 0.740 g (85% yield). 1H NMR (400 MHz, CDCl3) δ 7.43-7.36 (m, 5H), 7.28 (d, J=9.0 Hz, 2H), 6.85 (d, J=9.0 Hz, 2H), 5.17-5.12 (s, 2H), 4.29 (t, J=6.2 Hz, 2H), 3.63 (t, J=6.2 Hz, 2H).

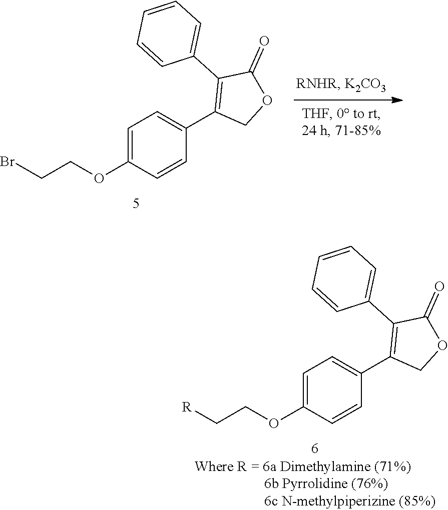

A mixture of amine (0.2 g, 1.2 equiv.) and K₂CO₃ (0.174 g, 3 equiv.) in dry THF (5 mL) was stirred for 10 min at 0° C. 4-(4-(2-bromoethoxy)phenyl)-3-phenylfuran-2(5H)-one (5) (0.150 g, 1 equiv.) was added to the reaction and the resulting mixture was stirred for 24 h at rt. The completion of the reaction was indicated by TLC. The reaction mixture was quenched with water (10 mL) and extracted with CH₂Cl₂ (3×20 mL). The organic layers were combined, dried over Na₂SO₄, and then filtered. The solvent was removed using a rotary evaporator, and the crude material was purified using preparative TLC, eluting withCH₂Cl₂:MeOH (10:1), v/v; to furnish to afford solid the desired product compound 6 in 71-85% yield.
Compound 6a (DW-379): ¹H NMR (400 MHz, CDCl₃) δ 7.46-7.32 (m, 5H), 7.27 (d, J=6.9 Hz, 3H), 6.85 (d, J=12.0 Hz, 2H), 5.16 (s, 2H), 4.05 (t, J=5.6 Hz, 2H), 2.73 (t, J=5.7 Hz, 2H), 2.33 (s, 6H).
Compound 6b (DW-372): ¹H NMR (400 MHz, CDCl3) δ 7.44-7.36 (m, 5H), 7.26 (d, J=9.1 Hz, 2H), 6.84 (d, J=9.1 Hz, 2H), 5.15 (s, 2H), 4.10 (t, J=6.3 Hz, 2H), 2.81 (t, J=6.3 Hz, 2H), 2.67-2.42 (m, 4H), 2.29 (s, 3H).
Compound 6c (DW-374): ¹H NMR (400 MHz, CDCl₃) δ 7.45-7.35 (d, J=7.7 Hz, 5H), 7.25 (d, J=9.1 Hz, 2H), 6.85 (d, J=9.1 Hz, 2H), 5.16 (s, 2H), 4.11 (t, J=5.9 Hz, 2H), 2.91 (t, J=5.9 Hz, 2H), 2.63 (s, 4H), 1.81 (s, 4H).

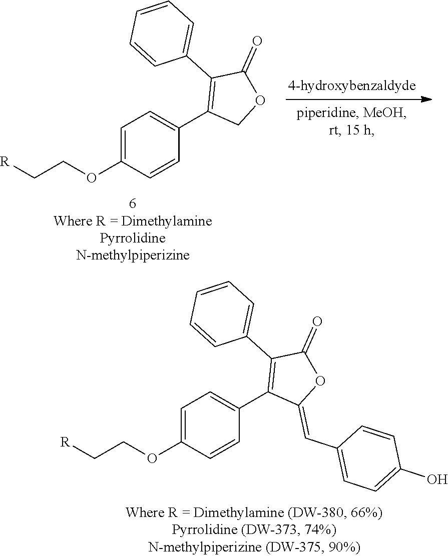

A mixture of Piperidine (1.1 equiv.), was added to a mixture of furan (6) (1 equiv.) and 4-hydroxy benzaldehyde (2 equiv.) were dissolved in 5 mL of methanol, and the reaction was stirred at rt for 15 h in a dark atmosphere. The completion of the reaction was indicated by TLC. The reaction was quenched with water (50 mL), extracted with CH₂C₁₂ (2×20 mL), and the combined organic layer was sequentially washed with a saturated solution of brine (30 mL). The organic layer was dried over Na₂SO₄, and then filtered. The solvent was removed using a rotary evaporator and the crude material was purified on silica gel column chromatography, eluting with CH₂Cl₂:Methanol (10:1), v/v, to afford solid the desired product in 66-90% yield.
DW-380: ¹H NMR (400 MHz, CDCl₃) δ 7.65 (d, J=6.8 Hz, 2H), 7.39 (d, J=9.9 Hz, 2H), 7.26-7.23 (m, 5H), 7.17 (d, J=8.8 Hz, 2H), 6.87 (d, J=3.5 Hz, 2H), 6.85 (d, J=3.5 Hz, 2H), 5.92 (s, 1H), 4.15 (t, J=5.4 Hz, 2H), 2.89 (t, J=5.4 Hz, 2H), 2.45 (s, 6H). ¹³C NMR (101 MHz, CDCl₃) δ 169.5, 159.4, 150.7, 146.5, 132.6, 130.6, 129.7, 129.1, 128.2, 122.7, 116.3, 114.8, 65.2, 58.1, 45.4.
DW-373: ¹H NMR (400 MHz, CDCl₃) δ 7.63 (d, J=8.5 Hz, 2H), 7.39 (d, J=9.9 Hz, 2H), 7.25-7.20 (m, 3H), 7.16 (d, J=8.0 Hz, 2H), 6.84 (m, 4H), 5.89 (s, 1H), 4.18 (t, 2H), 3.02 (t, 2H), 2.78 (s, 4H), 1.87 (s, 4H). 13C NMR (101 MHz, CDCl₃) δ 169.5, 159.5, 150.7, 146.6, 132.7, 130.7, 129.8, 129.2, 128.4, 125.1, 122.8, 116.4, 114.9, 114.0, 66.4, 55.0, 54.7, 29.8, 23.4.
DW-375: ¹H NMR (400 MHz, CD₃OD) δ 7.62 (d, J=6.6 Hz, 2H), 7.32 (s, 2H), 7.21 (m, 5H), 6.93 (d, J=6.6 Hz, 2H), 6.79 (d, J=8.8 Hz, 2H), 5.94 (s, 1H), 4.11 (t, J=5.6 Hz, 2H), 2.82 (t, J=5.6 Hz, 2H), 2.51 (m, 4H), 2.27 (s, 3H). ¹³C NMR (101 MHz, CD₃OD) δ 169.6, 159.5, 158.3, 150.8, 146.3, 132.6, 130.74-130.52, 129.6, 129.0, 128.3, 128.2, 124.9, 122.7, 115.8, 114.9, 114.3, 65.4, 56.8, 54.4, 52.9, 45.5, 29.6.

### Experimental Procedure & Characterization of DW-435 Family

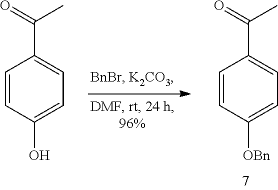

A solution of 4-hydroxy-acetopheone (8 g, 1 equiv.) in dry DMF (30 mL) was stirred under an argon atmosphere and cooled to 0° C. K₂CO₃ (2.02 g, 2 equiv.) was added and the mixture was allowed to warm up to rt and kept stirring at rt for 30 min. Subsequently, Benzyl bromide (1.9 1 mL, 1.2 equiv.) was added dropwise to the mixture, and the solution was stirred at rt for 24 h. After completion of the reaction as indicated by TLC, the reaction was quenched with water (50 mL) and extracted with Ethyl acetate (3×20 mL). The combined organic layer was dried over Na₂SO₄ and then filtered. The solvent was removed using a rotary evaporator, and the crude was purified by silica gel chromatography, eluting with Hexane:ethylacetate (12:1), v/v, to furnish the desired 1-(4-(benzyloxy)phenyl)ethan-1-one (7, DW-408). yield: 12.80 g (96%). 1H NMR (400 MHz, CDCl3) δ 7.93 (d, J=8.8 Hz, 2H), 7.45-7.31 (m, 5H), 7.00 (d, J=8.8 Hz, 2H), 5.13 (s, 2H), 2.55 (s, 3H).

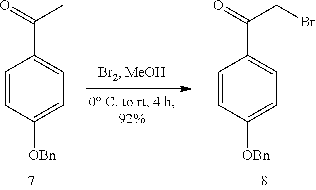 [SMILES: CC(=O)c1ccc(OCc2ccccc2)cc1 ; O=C(CBr)c1ccc(OCc2ccccc2)cc1]

Bromine (5.65 g, 1 equiv.) was added drop wise to a solution of 1-(4-(benzyloxy) phenyl) ethan-1-one (7) (8 g, lequiv.) in methanol (80 mL) was added at 0° C. and the mixture was allowed to warm up to rt and kept stirring at rt 15 h. After completion of the reaction as indicated by TLC, the reaction was quenched with water (100 mL) and Precipitate was filter and residue was with cold water ((2×100 mL) and yielded a pure product 8 (DW-409) as white solid in 10 g (92%). ¹H NMR (400 MHz, CDCl₃) δ 7.98 (d, J=8.8 Hz, 2H), 7.45-7.38 (m, 5H), 7.04 (d, J=8.8 Hz, 2H), 5.15 (s, 2H), 4.41 (s, 2H).

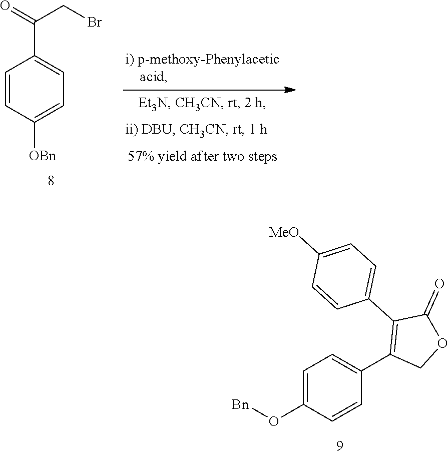 [SMILES: O=C(CBr)c1ccc(OCc2ccccc2)cc1 ; COc1ccc(C2=C(c3ccc(OCc4ccccc4)cc3)COC2=O)cc1]

To a stirred solution of substituted phenacyl bromide (8) (6 g 1 equiv.) and p-methoxy phenylacetic acid (3.265 g, 1 equiv.) in acetonitrile (50 ml) was added triethylamine (2.6 ml, 1 equiv.). The resultant mixture was stirred for 2 h at rt. The completion of the reaction as indicated by TLC. 1,8 Diazabicyclo[5.4.0]undec-7-ene (DBU) (2.97 g, 1 equiv.) was added and stirred for 1 h. Water (30 mL) was added and extracted with ethyl acetate (3×50 ml). The combined organic layer was dried over Na₂SO₄ and then filtered. The solvent was removed using a rotary evaporator, and the crude was purified by silica gel chromatography, eluting with Hexane:ethyl acetate (4:1), v/v, to furnish the desired 4-(4-(2-bromoethoxy) phenyl)-3-phenylfuran-2(5H)-one (9 DW-411) as solid 3.8 g (57% yield). ¹H NMR (400 MHz, CDCl₃) δ 7.45-7.30 (d, J=6.6 Hz, 11H), 6.92 (d, J=9.0 Hz, 2H), 5.17 (s, 2H), 5.09 (s, 2H), 3.83 (S, 3H).

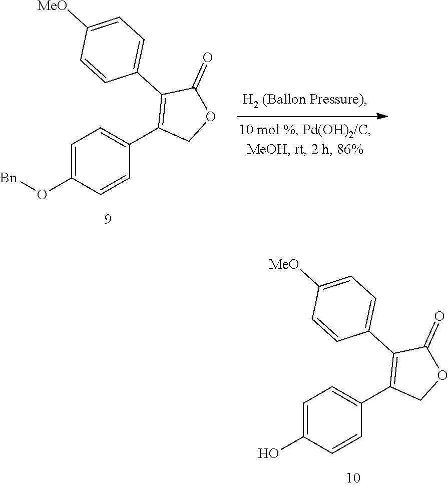 [SMILES: COc1ccc(C2=C(c3ccc(OCc4ccccc4)cc3)COC2=O)cc1 ; COc1ccc(C2=C(c3ccc(O)cc3)COC2=O)cc1]

Dissolve the 4-(4-(benzyloxy)phenyl)-3-(4-methoxyphenyl)furan-2(5H)-one (9) (5 g, 1 equiv.) in Methanol (10 mL) and catalytic amount of 10% Pd(OH)₂/C was added. The reaction was carried out under hydrogen (latm) for 2 h at rt. The reaction was monitored by TLC and after completion, the crude product was filtered through a Celite® bed and The solvent was removed using a rotary evaporator, and the crude was purified by silica gel chromatography, eluting with Hexane:ethyl acetate (5:5), v/v, to furnish the desired 4-(4-hydroxyphenyl)-3-(4-methoxyphenyl) furan-2(5H)-one (10 DW-412) as solid 3.3 g (86% yield). ¹H NMR (400 MHz, cd3od) δ 7.05 (d, J=6.7 Hz, 2H), 6.95 (dd, J=8.4, 3.4 Hz, 2H), 6.64 (d, 2H), 6.46 (d, J=9.0 Hz, 2H), 4.91 (s, 2H), 3.54 (s, 3H).

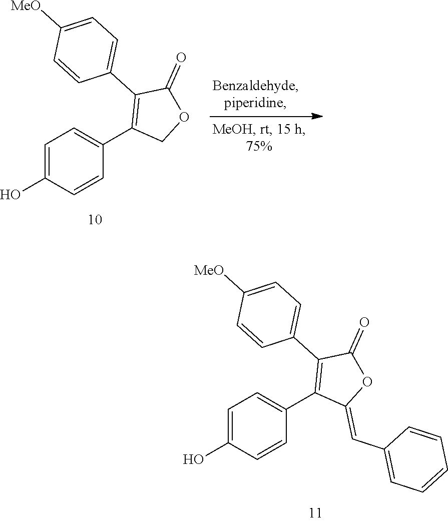 [SMILES: COc1ccc(C2=C(c3ccc(O)cc3)COC2=O)cc1 ; COc1ccc(C2=C(c3ccc(O)cc3)/C(=C/c3ccccc3)OC2=O)cc1]

A mixture of Piperidine (0.772 mL equiv.), was added to a mixture of furan (10) (2 g 1 equiv.) and benzaldehyde (1.503 g, 2 equiv.) were dissolved in 20 mL of methanol and the reaction was stirred at rt for 15 h in a dark atmosphere. The completion of the reaction was indicated by TLC. The reaction was quenched with water (50 mL), extracted with ethyl acetate (2×20 mL), and the combined organic layer was sequentially washed with a saturated solution of brine (30 mL). The organic layer was dried over Na₂SO₄, and then filtered. The solvent was removed using a rotary evaporator and the crude material was purified on silica gel column chromatography, eluting with Hexane:ethyl acetate (8:2), v/v, to afford solid the desired product 11(DW-413) in 1.98 g (75% yield). ¹H NMR (400 MHz, CD₃OD) δ 7.98 (d, J=8.5 Hz, 2H), 7.72 (d, J=2.1 Hz, 2H), 7.40-7.29 (m, 5H), 7.14 (d, 2H), 6.87 (d, J=8.7 Hz, 2H), 6.76 (d, J=9.1 Hz, 2H), 5.99 (s, 1H), 3.73 (s, 3H).

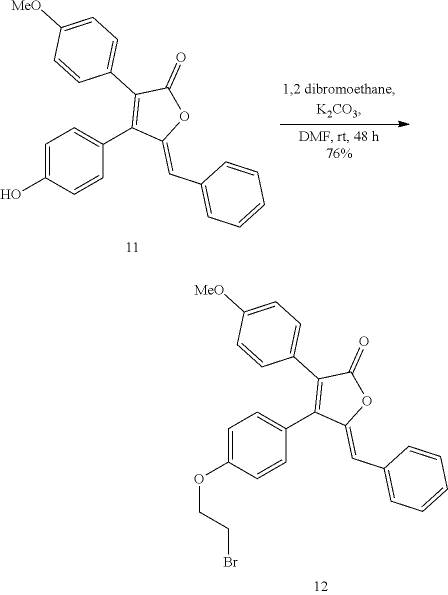 [SMILES: COc1ccc(C2=C(c3ccc(O)cc3)/C(=C/c3ccccc3)OC2=O)cc1 ; COc1ccc(C2=C(c3ccc(OCCBr)cc3)/C(=C/c3ccccc3)OC2=O)cc1]

A solution of (Z)-5-benzylidene-4-(4-hydroxyphenyl)-3-(4-methoxyphenyl)furan-2(5H)-one (11) (2 g, 1 equiv.) in dry DMF (20 mL) was stirred under an argon atmosphere and cooled to 0° C. K₂CO₃ (1.280 g, 2 equiv.) was added and the mixture was allowed to warm up to rt and kept stirring at rt for 30 min. Subsequently, 1, 2-dibromoethane (1.60 mL, 3 equiv.) was added dropwise to the mixture, and the solution was reflux for 48 h. After completion of the reaction as indicated by TLC, the reaction was quenched with water (50 mL) and extracted with Ethyl acetate (3×20 mL). The combined organic layer was dried over Na₂SO₄ and then filtered. The solvent was removed using a rotary evaporator, and the crude was purified by silica gel chromatography, eluting with Hexane:ethylacetate (9:1), v/v, to furnish the desired compound 12(DW-417) in 1.98 g (76% yield). ¹H NMR (400 MHz, CDCl₃) δ 7.79 (d, J=7.6 Hz, 2H), 7.42-7.29 (m, 7H), 7.01 (d, J=8.9 Hz, 2H), 6.82 (d, J=9.1 Hz, 2H), 5.96 (s, 1H), 4.36 (t, J=6.1 Hz, 2H), 3.76 (s, 3H), 3.70 (t, J=6.2 Hz, 2H).

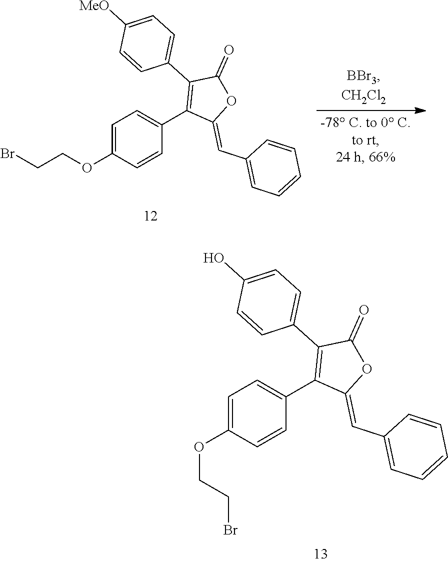 [SMILES: COc1ccc(C2=C(c3ccc(OCCBr)cc3)/C(=C/c3ccccc3)OC2=O)cc1 ; O=C1O/C(=C\c2ccccc2)C(c2ccc(OCCBr)cc2)=C1c1ccc(O)cc1]

Dissolve (Z)-5-benzylidene-4-(4-(2-bromoethoxy)phenyl)-3-(4-methoxyphenyl)furan-2(5H)-one(12) (1 g, 1 equiv.) in the Dry DCM (5 mL) under a argon atmosphere and cool the reaction mixture to −78° C. and stir the reaction mixture 15 minutes for same temp. BBr₃ (0.630 g, 1.2 equiv.) was added dropwise to the mixture, and allow the reaction mixture 0° C. then to room temperature and stirred reaction mixture at rt for 24 h. After completion of the reaction as indicated by TLC, the reaction mixture was quenched with water (10 mL) and extracted with CH₂Cl₂ (2×20 mL). The organic phase was dried over Na₂SO₄ and then filtered. The solvent was removed using a rotary evaporator, and the crude was purified by silica gel chromatography, eluting with Hexane:ethylacetate (6:4), v/v, to furnish the desired compound 13 (DW-422) in 0.65 g (66% yield). ¹H NMR (400 MHz, CD₃OD) δ 7.72 (d, J=7.6 Hz, 2H), 7.32 (d, J=7.2 Hz, 2H), 7.25 (s, 5H), 6.96 (d, J=9.1 Hz, 2H), 6.68 (d, J=10.2 Hz, 1H), 5.92 (s, 1H), 4.32 (t, J=6.1 Hz, 2H), 3.65 (t, J=6.1 Hz, 2H).

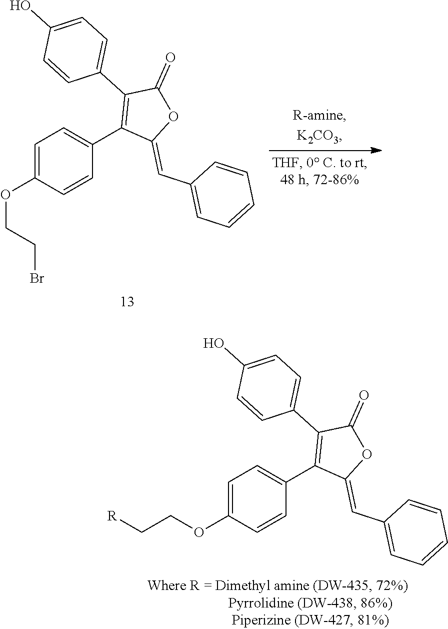

A mixture of amine (1.2 equiv.) and K₂CO₃ (59 mg, 2 equiv.) in dry THF (5 mL) was stirred for 10 min at 0° C. (Z)-5-benzylidene-4-(4-(2-bromoethoxy)phenyl)-3-(4-hydroxyphenyl)furan-2(5H)-one (13) (0.1 g, 1 equiv.) was added to the reaction and the resulting mixture was stirred for 48 h at rt. The completion of the reaction was indicated by TLC. The reaction mixture was quenched with water (10 mL) and extracted with CH₂Cl₂ (3×20 mL). The organic layers were combined, dried over Na₂SO₄, and then filtered. The solvent was removed using a rotary evaporator, and the crude material was purified using preparative TLC, eluting with CH₂Cl₂:MeOH (10:1), v/v; to furnish to afford solid the desired product in 72-86% yield.
DW-427: ¹H NMR (400 MHz, CD₃OD) δ 7.74 (d, J=5.7 Hz, 2H), 7.34 (d, J=12.9 Hz, 2H), 7.26 (m, 5H), 6.97 (d, J=8.9 Hz, 2H), 6.69 (d, J=12.9 Hz, 2H), 5.96 (s, 1H), 4.14 (t, 2H), 2.85 (t, J=7.1, 4.9 Hz, 2H), 2.56 (s, 4H), 2.31 (s, 3H). ¹³C NMR (101 MHz, CD₃OD) δ 169.6, 159.5, 158.3, 150.8, 146.3, 132.6, 130.7, 130.5, 129.6, 129.0, 128.3, 128.2, 124.9, 122.7, 115.8, 114.9, 114.3, 65.4, 56.8, 54.4, 52.9, 45.5, 29.6.
DW-435: ¹H NMR (400 MHz, CDCl₃) δ 7.77 (d, J=7.9 Hz, 2H), 7.36 (t, J=7.7 Hz, 3H), 7.29 (d, 2H), 7.18 (d, J=8.8 Hz, 2H), 6.74 (d, 2H) 6.70 (d, 2H), 5.95 (s, 1H), 4.13 (t, J=5.3 Hz, 2H), 2.87 (t, J=5.3 Hz, 2H), 2.42 (s, 6H). ¹³C NMR (101 MHz, CDCl₃) δ 169.5, 159.4, 150.7, 146.5, 132.6, 130.6, 129.7, 129.1, 128.2, 122.7, 116.3, 114.8, 65.2, 58.1, 45.4.
DW-438: 1H NMR (400 MHz, DMSO-d6) δ 7.78 (d, J=8.0 Hz, 2H), 7.44 (t, J=7.7 Hz, 2H), 7.34 (d, J=11.8 Hz, 3H), 7.22 (d, J=8.8 Hz, 2H), 7.11 (d, J=8.8 Hz, 2H), 6.72 (d, J=8.8 Hz, 2H), 6.02 (s, 1H), 4.25 (t, 2H), 3.12 (t, 2H), 2.85 (m, 4H), 1.80 (m, 4H). 13C NMR (101 MHz, DMSO-d6) δ 169.5, 159.5, 150.7, 146.6, 132.7, 130.7, 129.8, 129.2, 128.4, 125.1, 122.8, 116.4, 114.9, 114.0, 66.4, 55.0, 54.7, 29.8, 23.4.

### Experimental Procedure & Characterization of DW-749, DW-759, and DW-782

### Experimental Procedure & Characterization of DW-749

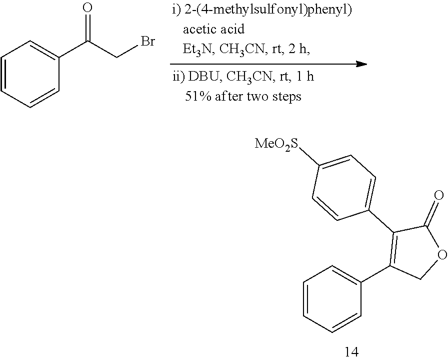 [SMILES: O=C(CBr)c1ccccc1 ; CS(=O)(=O)c1ccc(C2=C(c3ccccc3)COC2=O)cc1]

To a stirred solution of phenacyl bromide (0.8 g 1 equiv.) and 2-(4-(methylsulfonyl)phenyl) acetic acid (0.860 g, 1 equiv.) in acetonitrile (10 ml) was added triethylamine (0.534 ml, 1 equiv.). The resultant mixture was stirred for 2 h at rt. The completion of the reaction as indicated by TLC. 1,8 Diazabicyclo[5.4.0]undec-7-ene (DBU) (0.611 g, 1 equiv.) was added and stirred for 1 h. Water (20 mL) was added and extracted with ethyl acetate (3×20 ml). The combined organic layer was dried over Na₂SO₄ and then filtered. The solvent was removed using a rotary evaporator, and the crude was purified by silica gel chromatography, eluting with Hexane:ethyl acetate (3:2), v/v, to furnish the desired product 14 (DW-748) as solid 0.62 g (51% yield). ¹H NMR (400 MHz, CDCl₃) δ 7.96 (d, J=8.5 Hz, 2H), 7.66 (d, J=8.6 Hz, 2H), 7.45 (d, 1H), 7.41 (d, J=7.7 Hz, 2H), 7.28 (d, J=9.2 Hz, 3H), 5.25 (s, 2H), 3.03 (s, 3H).

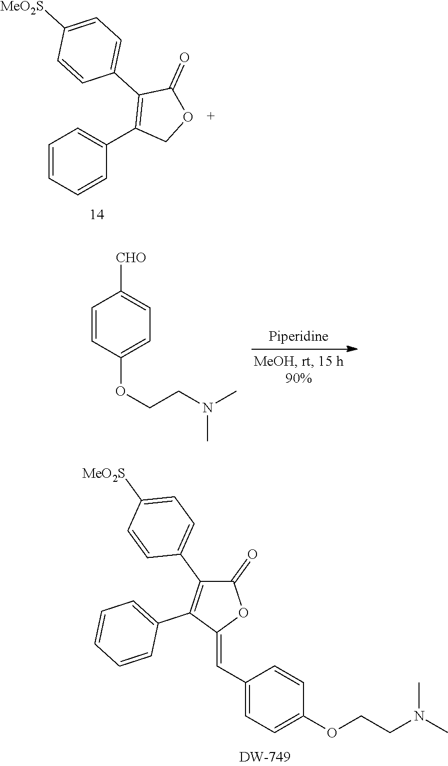 [SMILES: CS(=O)(=O)c1ccc(C2=C(c3ccccc3)COC2=O)cc1 ; CN(C)CCOc1ccc(C=O)cc1 ; CN(C)CCOc1ccc(/C=C2\OC(=O)C(c3ccc(S(C)(=O)=O)cc3)=C2c2ccccc2)cc1]

A mixture of Piperidine (0.069 mL 1 equiv.), was added to a mixture of furan (14) (0.2 g 1 equiv.) and 4-(2-(dimethylamino)ethoxy)benzaldehyde (0.244 g, 2 equiv.) were dissolved in 10 mL of methanol and the reaction was stirred at rt for 15 h in a dark atmosphere. The completion of the reaction was indicated by TLC. The reaction was quenched with water (30 mL), extracted with ethyl acetate (2×20 mL), and the combined organic layer was sequentially washed with a saturated solution of brine (30 mL). The organic layer was dried over Na₂SO₄, and then filtered. The solvent was removed using a rotary evaporator and the crude material was purified on silica gel column chromatography, eluting with Hexane:ethyl acetate (1:1), v/v, to afford solid the desired product in 0.281 g (90% yield). ¹H NMR (400 MHz, CDCl₃) δ 7.23 (m, 4H), 7.05 (d, J=8.4 Hz, 2H), 6.96 (d, J=7.8 Hz, 3H), 6.77 (d, J=6.1 Hz, 2H), 6.39 (d, J=10.5 Hz, 2H), 5.49 (s, 1H), 3.55 (t, 2H), 2.46 (s, 3H), 2.19 (t, 2H), 1.78 (s, 6H). ¹³C NMR (101 MHz, CDCl₃) δ 168.3, 149.3, 148.8, 144.1, 141.9, 136.1, 135.3, 133.1, 130.8, 129.9, 129.4, 128.9, 128.0, 122.4, 120.5, 115.7, 112.5, 65.2, 58.0, 45.3, 43.8.

### Experimental Procedure & Characterization of DW-759

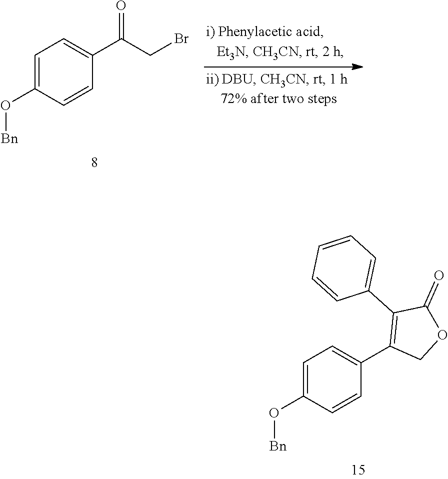 [SMILES: O=C(CBr)c1ccc(OCc2ccccc2)cc1 ; O=C1OCC(c2ccc(OCc3ccccc3)cc2)=C1c1ccccc1]

To a stirred solution of 1-(4-(benzyloxy)phenyl)-2-bromoethan-1-one (8) (1 g 1 equiv.) and phenyl) acetic acid (0.446 g, 1 equiv.) in acetonitrile (10 ml) was added triethylamine (0.435 ml, 1 equiv.). The resultant mixture was stirred for 2 h at rt. The completion of the reaction as indicated by TLC. 1,8 Diazabicyclo[5.4.0]undec-7-ene (DBU) (0.498 g, 1 equiv.) was added and stirred for 1 h. Water (20 mL) was added and extracted with ethyl acetate (3×20 ml). The combined organic layer was dried over Na₂SO₄ and then filtered. The solvent was removed using a rotary evaporator, and the crude was purified by silica gel chromatography, eluting with Hexane:ethyl acetate (5:1), v/v, to furnish the desired product 15 (DW-742) as solid 0.81 g (72% yield). ¹H NMR (400 MHz, CDCl₃) δ 7.45-7.34 (m, 10H), 7.27 (d, J=9.1 Hz, 2H), 6.90 (d, J=9.0 Hz, 2H), 5.14 (s, 2H), 5.05 (s, 2H).

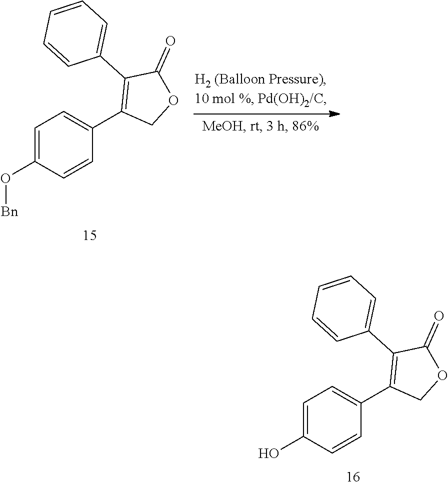 [SMILES: O=C1OCC(c2ccc(OCc3ccccc3)cc2)=C1c1ccccc1 ; O=C1OCC(c2ccc(O)cc2)=C1c1ccccc1]

Dissolve the 4-(4-(benzyloxy)phenyl)-3-phenylfuran-2(5H)-one (15) (0.8 g, 1 equiv.) in Methanol (10 mL) and catalytic amount of 10% Pd(OH)2/C was added. The reaction was carried out under hydrogen (latm) for 3 h at rt. The reaction was monitored by TLC and after completion, the crude product was filtered through a Celite® bed and The solvent was removed using a rotary evaporator, and the crude product was purified by silica gel chromatography, eluting with Hexane:ethyl acetate (5:5), v/v, to furnish the desired 4-(4-hydroxyphenyl)-3-phenylfuran-2(5H)-one (16 DW-743) as white solid 0.51 g (86% yield). ¹H NMR (400 MHz, CDCl₃) δ 7.35-7.21 (m, 5H), 7.12 (d, J=6.6 Hz, 2H), 6.66 (d, J=8.9 Hz, 2H), 5.12 (s, 2H).

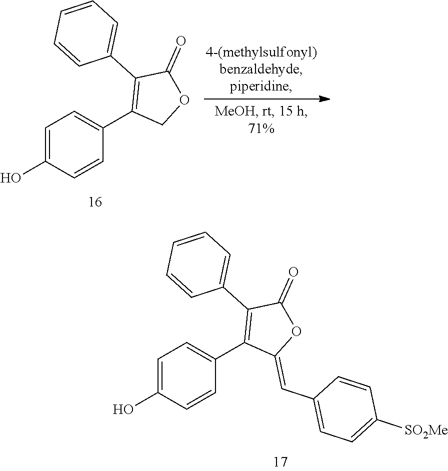 [SMILES: O=C1OCC(c2ccc(O)cc2)=C1c1ccccc1 ; CS(=O)(=O)c1ccc(/C=C2\OC(=O)C(c3ccccc3)=C2c2ccc(O)cc2)cc1]

A mixture of Piperidine (0.061 mL 1 equiv.), was added to a mixture of 4-(4-hydroxyphenyl)-3-phenylfuran-2(5H)-one (16) (0.2 g 1 equiv.) and 4-(methylsulfonyl) benzaldehyde (0.234 g, 2 equiv.) were dissolved in 10 mL of methanol and the reaction was stirred at rt for 15 h in a dark atmosphere. The completion of the reaction was indicated by TLC. The reaction was quenched with water (30 mL), extracted with ethyl acetate (2×20 mL), and the combined organic layer was dried over Na₂SO₄, and then filtered. The solvent was removed using a rotary evaporator and the crude material was purified on silica gel column chromatography, eluting with Hexane:ethyl acetate (3:2), v/v, to afford solid the desired product 17 (DW-746) in 0.19 g (71% yield). ¹H NMR (400 MHz, CDCl₃) δ 8.01-7.88 (m, 4H), 7.44 (m, 2H), 7.31 (s, 3H), 7.20 (d, J=8.8 Hz, 2H), 6.94 (d, J=8.7 Hz, 2H), 6.13 (s, 1H), 3.12 (s, 3H).

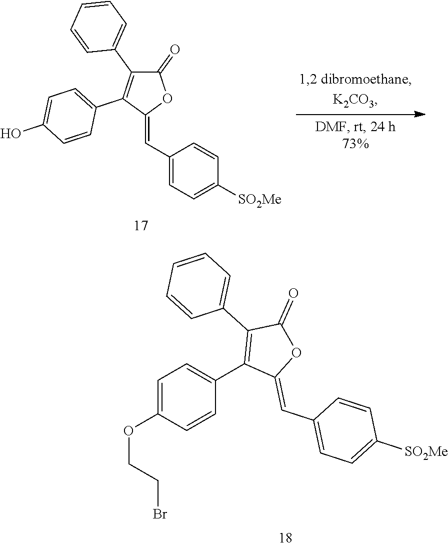 [SMILES: CS(=O)(=O)c1ccc(/C=C2\OC(=O)C(c3ccccc3)=C2c2ccc(O)cc2)cc1 ; CS(=O)(=O)c1ccc(/C=C2\OC(=O)C(c3ccccc3)=C2c2ccc(OCCBr)cc2)cc1]

A solution of (Z)-4-(4-hydroxyphenyl)-5-(4-(methylsulfonyl)benzylidene)-3-phenylfuran-2(5H)-one (17) (0.2 g, 1 equiv.) in dry DMF (4 mL) was stirred under an argon atmosphere and cooled to 0° C. K₂CO₃ (0.132 g, 2 equiv.) was added and the mixture was allowed to warm up to rt and kept stirring at rt for 30 min. Subsequently, 1, 2-dibromoethane (0.123 mL, 3 equiv.) was added dropwise to the mixture, and the solution was reflux for 24 h. After completion of the reaction as indicated by TLC, the reaction was quenched with water (50 mL) and extracted with Ethyl acetate (3×20 mL). The combined organic layer was dried over Na2SO4 and then filtered. The solvent was removed using a rotary evaporator, and the crude was purified by silica gel chromatography, eluting with Hexane:ethylacetate (1:1), v/v, to furnish the desired compound 18 (DW-758) in 0.18 g (73% yield). ¹H NMR (400 MHz, CDCl₃) δ 7.92 (m, 4H), 7.42 (m, 2H), 7.28 (m, 5H), 7.01 (d, J=8.9 Hz, 2H), 6.02 (s, 1H), 4.36 (t, J=6.1 Hz, 2H), 3.70 (d, J=6.1 Hz, 2H), 3.06 (s, 3H).

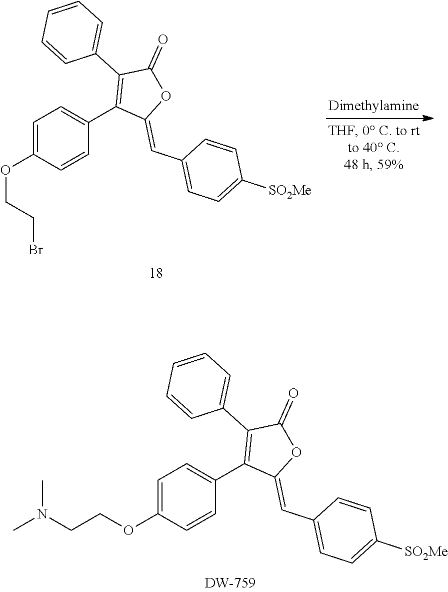 [SMILES: CS(=O)(=O)c1ccc(/C=C2\OC(=O)C(c3ccccc3)=C2c2ccc(OCCBr)cc2)cc1 ; CN(C)CCOc1ccc(C2=C(c3ccccc3)C(=O)O/C2=C\c2ccc(S(C)(=O)=O)cc2)cc1]

A mixture of dimethyl amine (0.068 g, 5 equiv.) in dry THF (5 mL) was stirred for 10 min at 0° C. (Z)-4-(4-(2-bromoethoxy)phenyl)-5-(4-(methylsulfonyl)benzylidene)-3-phenylfuran-2(5H)-one(18) (0.160 g, 1 equiv.) was added to the reaction and the resulting mixture was stirred for rt to 40° C. for 48 h. The completion of the reaction was indicated by TLC. The reaction mixture was quenched with water (10 mL) and extracted with CH₂Cl₂ (3×20 mL). The organic layers were combined, dried over Na₂SO₄, and then filtered. The solvent was removed using a rotary evaporator, and the crude material was purified using preparative TLC, eluting with CH₂Cl₂:MeOH (10:1), v/v; to furnish to afford solid the desired product in 0.072 g (59% yield). ¹H NMR (400 MHz, CDCl₃) δ 7.90 (m, 5H), 7.39 (d, J=9.6 Hz, 2H), 7.26 (s, 6H), 6.98 (d, J=8.6 Hz, 2H), 5.98 (s, 1H), 4.09 (t, 2H), 3.04 (s, 3H), 2.76 (t, 2H), 2.35 (s, 6H). ¹³C NMR (101 MHz, CDCl₃) δ 168.3, 149.3, 148.8, 144.1, 141.9, 136.1, 135.3, 133.1, 130.8, 129.9, 129.4, 128.9, 128.0, 122.4, 120.5, 115.7, 112.5, 65.2, 58.0, 45.3, 43.8.

### Experimental Procedure & Characterization of DW-782

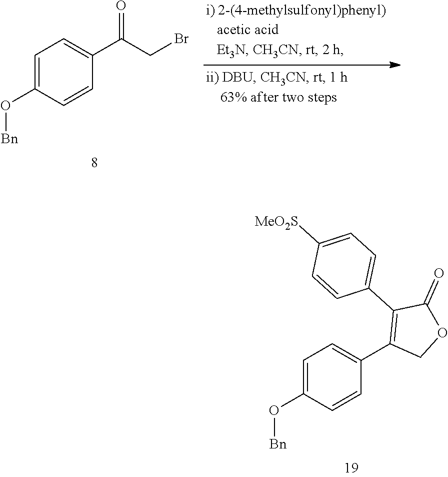 [SMILES: O=C(CBr)c1ccc(OCc2ccccc2)cc1 ; CS(=O)(=O)c1ccc(C2=C(c3ccc(OCc4ccccc4)cc3)COC2=O)cc1]

To a stirred solution of 1-(4-(benzyloxy)phenyl)-2-bromoethan-1-one (8) (0.750 g 1 equiv.) and 2-(4-(methylsulfonyl)phenyl) acetic acid (0.701 g, 1 equiv.) in acetonitrile (20 ml) was added triethylamine (0.392 ml, 1.2 equiv.). The resultant mixture was stirred for 2 h at rt. The completion of the reaction as indicated by TLC. 1,8 Diazabicyclo[5.4.0]undec-7-ene (DBU) (0.373 g, 1 equiv.) was added and stirred for 1 h. Water (20 mL) was added and extracted with ethyl acetate (3×20 ml). The combined organic layer was dried over Na₂SO₄ and then filtered. The solvent was removed using a rotary evaporator, and the crude was purified by silica gel chromatography, eluting with Hexane:ethyl acetate (3:2), v/v, to furnish the desired product 19 (DW-778) as solid 0.63 g (63% yield). ¹H NMR (400 MHz, CDCl₃) δ 7.97 (m, 2H), 7.65 (d, J=14.0 Hz, 2H), 7.52 (s, 1H), 7.46-7.32 (m, 5H), 7.25-7.21 (m, 1H), 6.90 (d, J=1.7 Hz, 1H), 6.85 (d, J=1.7 Hz, 1H) 5.2 (s, 2H), 5.08 (s, 2H), 3.07 (s, 3H).

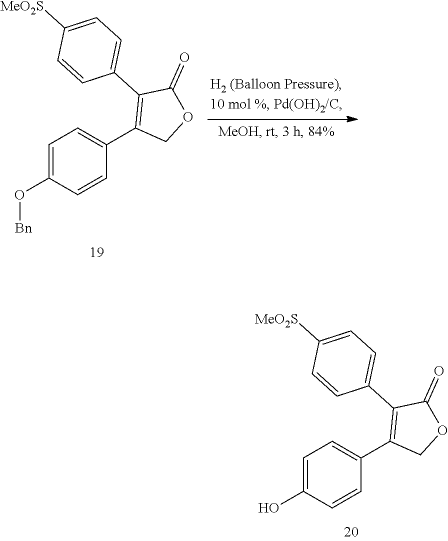 [SMILES: CS(=O)(=O)c1ccc(C2=C(c3ccc(OCc4ccccc4)cc3)COC2=O)cc1 ; CS(=O)(=O)c1ccc(C2=C(c3ccc(O)cc3)COC2=O)cc1]

Dissolve the 4-(4-(benzyloxy)phenyl)-3-(4-(methylsulfonyl)phenyl)furan-2(5H)-one (19) (0.180 g, 1 equiv.) in Methanol (4 mL) and catalytic amount of 10% Pd(OH)₂/C was added. The reaction was carried out under hydrogen (latm) for 3 h at rt. The reaction was monitored by TLC and after completion, the crude product was filtered through a Celite® bed and The solvent was removed using a rotary evaporator, and the crude product was purified by silica gel chromatography, eluting with Hexane:ethyl acetate (5:5), v/v, to furnish the desired 4-(4-hydroxyphenyl)-3-(4-(methylsulfonyl)phenyl)furan-2(5H)-one (20 DW-779) as white solid 0.12 g (84% yield). ¹H NMR (400 MHz, CDCl₃) δ 7.72 (d, 2H), 7.44 (d, 2H), 6.95 (d, 2H), 6.56 (d, 2H), 5.01 (s, 2H), 2.84 (s, 3H).

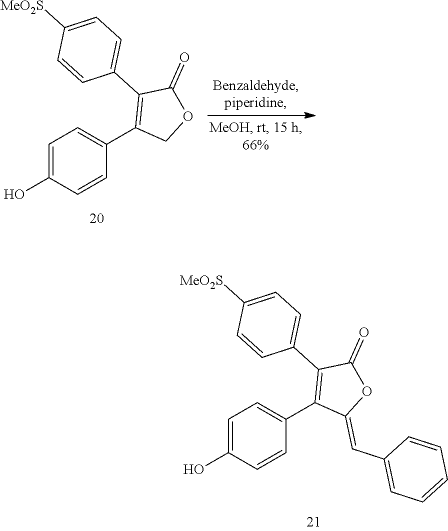 [SMILES: CS(=O)(=O)c1ccc(C2=C(c3ccc(O)cc3)COC2=O)cc1 ; CS(=O)(=O)c1ccc(C2=C(c3ccc(O)cc3)/C(=C/c3ccccc3)OC2=O)cc1]

A mixture of Piperidine (0.032 mL 1 equiv.), was added to a mixture of 4-(4-hydroxyphenyl)-3-(4-(methylsulfonyl)phenyl)furan-2(5H)-one (20) (0.1 g 1 equiv.) and benzaldehyde (0.064 g, 2 equiv.) were dissolved in 5 mL of methanol and the reaction was stirred at rt for 15 h in a dark atmosphere. The completion of the reaction was indicated by TLC. The reaction was quenched with water (20 mL), extracted with ethyl acetate (2×20 mL), and the combined organic layer was dried over Na₂SO₄, and then filtered. The solvent was removed using a rotary evaporator and the crude material was purified on silica gel column chromatography, eluting with Hexane:ethyl acetate (3:2), v/v, to afford solid the desired product 21 (DW-780) in 0.08 g (66% yield). ¹H NMR (400 MHz, CDCl₃) δ 7.76 (m, 4H), 7.59 (d, J=8.7 Hz, 2H), 7.34 (m, 3H), 7.12 (d, 2H), 6.88 (d, J=8.7 Hz, 2H), 6.12 (s, 1H), 3.00 (s, 3H).

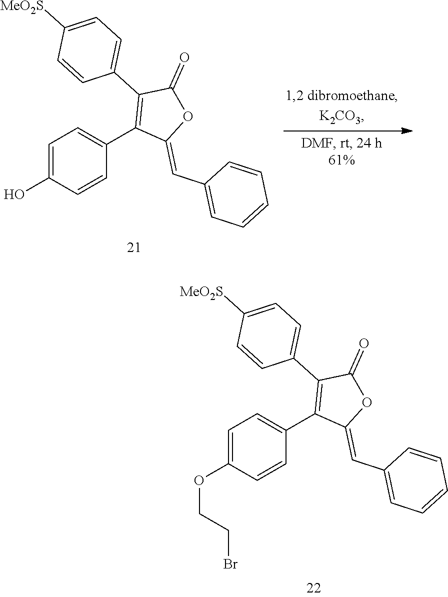 [SMILES: CS(=O)(=O)c1ccc(C2=C(c3ccc(O)cc3)/C(=C/c3ccccc3)OC2=O)cc1 ; CS(=O)(=O)c1ccc(C2=C(c3ccc(OCCBr)cc3)/C(=C/c3ccccc3)OC2=O)cc1]

A solution of (Z)-5-benzylidene-4-(4-hydroxyphenyl)-3-(4-(methylsulfonyl)phenyl)furan-2(5H)-one (21) (80 mg, 1 equiv.) in dry DMF (2 mL) was stirred under an argon atmosphere and cooled to 0° C. K₂CO₃ (39 mg, 1.5 equiv.) was added and the mixture was allowed to warm up to rt and kept stirring at rt for 30 min. Subsequently, 1, 2-dibromoethane (0.049 mL, 3 equiv.) was added dropwise to the mixture, and the solution was reflux for 24 h. After completion of the reaction as indicated by TLC, the reaction was quenched with water (50 mL) and extracted with Ethyl acetate (3×20 mL). The combined organic layer was dried over Na₂SO₄ and then filtered. The solvent was removed using a rotary evaporator, and the crude was purified by silica gel chromatography, eluting with Hexane:ethylacetate (4:2), v/v, to furnish the desired compound 22 (DW-781) in 61 mg (61% yield). ¹H NMR (400 MHz, CDCl₃) δ 7.87-7.78 (m, 4H), 7.62 (d, J=7.1 Hz, 2H), 7.39 (d, J=12.8 Hz, 3H), 7.26 (d, 2H), 7.03 (d, J=8.8 Hz, 2H), 6.12 (s, 1H), 4.37 (t, J=6.8 Hz, 2H), 3.70 (t, J=6.1 Hz, 2H), 3.04 (s, 3H).

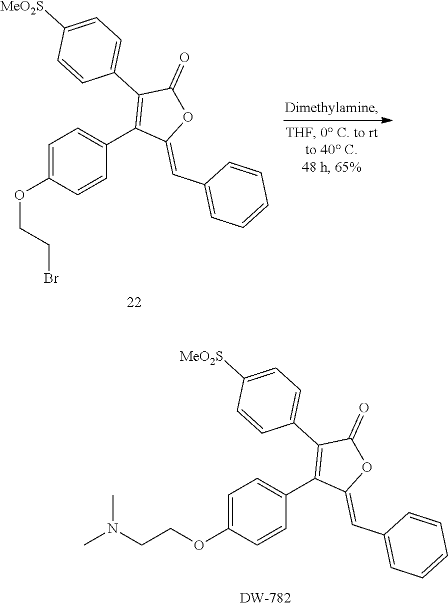 [SMILES: CS(=O)(=O)c1ccc(C2=C(c3ccc(OCCBr)cc3)/C(=C/c3ccccc3)OC2=O)cc1 ; CN(C)CCOc1ccc(C2=C(c3ccc(S(C)(=O)=O)cc3)C(=O)O/C2=C\c2ccccc2)cc1]

A mixture of dimethyl amine (25 mg, 5 equiv.) in dry THF (1 mL) was stirred for 10 min at 0° C. (Z)-5-benzylidene-4-(4-(2-bromoethoxy)phenyl)-3-(4-(methylsulfonyl)phenyl)furan-2(5H)-one (22) (60 mg, 1 equiv.) was added to the reaction and the resulting mixture was stirred for rt to 40° C. for 48 h. The completion of the reaction was indicated by TLC. The reaction mixture was quenched with water (10 mL) and extracted with CH₂Cl₂ (3×10 mL). The organic layers were combined, dried over Na₂SO₄, and then filtered. The solvent was removed using a rotary evaporator, and the crude material was purified using preparative TLC, eluting with CH₂Cl₂:MeOH (10:1), v/v; to furnish to afford the desired product as a solid in 36 mg (65% yield). ¹H NMR (400 MHz, CDCl₃) δ 7.85-7.77 (m, 4H), 7.61 (d, J=8.8 Hz, 2H), 7.41-7.32 (m, 3H), 7.23 (d, J=8.9 Hz, 2H), 7.02 (d, J=8.9 Hz, 2H), 6.11 (s, 1H), 4.13 (t, J=5.6 Hz, 2H), 3.02 (s, 3H), 2.78 (t, J=5.7 Hz, 2H), 2.36 (s, 6H). ¹³C NMR (101 MHz, CDCl₃) δ 168.3, 149.3, 148.8, 144.1, 141.9, 136.1, 135.3, 133.1, 130.8, 129.9, 129.4, 128.9, 128.0, 122.4, 120.5, 115.7, 112.5, 65.2, 58.0, 45.3, 43.8.

### Example 3—Biological Data

In this present disclosure, the cytotoxic activity of butenolide-based compounds were evaluated against various cancer cell lines using MTT assays. The MTT assay measures cellular metabolic activity as an indicator of cell viability, proliferation, and cytotoxicity. In this colorimetric assay, metabolically active cells reduce a yellow tetrazolium dye (3-(4,5-dimethylthiazol-2-yl)-2,5-diphenyltetrazolium bromide) into an insoluble purple formazan product through the action of mitochondria-related enzymes. The intensity of the resulting purple color, measured by spectrophotometry, is directly proportional to the number of viable cells. By testing compounds across a range of concentrations, dose-response curves were generated from which IC₅₀ values were determined as the concentration required to reduce cell viability by 50% compared to control cells. Tamoxifen (TAM) served as the control compound for comparison, and Vero cells, an immortalized lineage of kidney epithelial cells, served as a control cell line to assess selectivity for cancer cells over non-cancerous cells.

Table 1 presents IC₅₀ values for butenolide-based compounds tested against A549 lung cancer cells, Du145 and LNCaP prostate cancer cells, HepG2 liver cancer cells, SK-1EP-1 endothelial cancer cells, MCF-7 breast cancer cells, MDA-MB-231 triple-negative breast cancer cells, and Vero control cells. The results demonstrated that several butenolide-based compounds exhibited potent cytotoxic activity against the diverse panel of cancer cell lines. Compounds showing lower IC₅₀ values than the tamoxifen (Tam) control indicated enhanced cytotoxic potency, while compounds with higher IC₅₀ values against Vero cells relative to the cancer cell lines suggested favorable selectivity profiles. The differential activity observed across the cell lines provided insight into the mechanism of action and potential therapeutic applications, as the panel included hormone-responsive cell lines such as MCF-7 and LNCaP as well as hormone-independent cell lines such as MIDA-MB-231 and Du145.

TABLE 1
IC₅₀ values obtained based on MTT cell viability assay

| | | Cell Line and IC₅₀ Value (μM) | | | | | | | |
|---|---|---|---|---|---|---|---|---|---|
| | | | | | | | | MDA- | |
| | | | DU- | | | SK- | | MB- | |
| S/No. | Compound | A549 | 145 | LNCaP | HepG2 | HEP-1 | MCF-7 | 231 | Vero |
| 1 | TAM | 14.37 ± 1.8 | 22.03 ± 3.7 | 18.01 ± 4.1 | 11.58 ± 0.9 | 15.64 ± 3.7 | 23.46 ± 2.3 | 15.41 ± 3.8 | 22.15 ± 1.0 |
| 2 | DW-159 | 87.08 | 63.85 | 30.75 | 19.02 | 30.25 | >100 | 31.52 | >100 |
| 3 | DW-165 | 14.37 ± 0.8 | 10.01 ± 1.4 | 4.65 ± 2.4 | 2.17 ± 0.6 | 3.65 ± 1.9 | 3.64 ± 0.5 | 5.35 ± 0.9 | 7.99 ± 0.2 |
| 4 | DW-160 | >100 | >100 | >50 | >100 | >100 | >100 | >100 | >100 |
| 5 | DW-176 | 5.25 ± 1.5 | 4.87 ± 0.5 | 2.97 ± 0.7 | 2.35 ± 0.3 | 2.81 ± 0.1 | 4.27 ± 1.2 | 3.86 ± 0.9 | 4.95 ± 1.4 |
| 6 | DW-178 | 10.95 ± 1.2 | 5.89 ± 1.97 | 3.31 ± 1.1 | 2.50 ± 0.2 | 3.14 ± 1.2 | 4.05 ± 1.6 | 9.96 ± 0.9 | 7.03 ± 2.6 |
| 7 | DW-182 | 14.13 ± 1.7 | 11.54 ± 3.1 | 3.04 ± 0.9 | 3.09 ± 0.7 | 5.3 ± 0.9 | 8.69 ± 2.7 | 5.80 ± 0.1 | 12.25 ± 2.9 |
| 8 | DW-183 | 11.97 ± 1.7 | 11.99 | 5.01 ± 2.0 | 4.50 ± 0.1 | 7.89 ± 2.5 | 7.94 ± 1.2 | 7.96 ± 2.8 | 6.48 ± 0.7 |
| 9 | DW-187 | 7.94 ± 3.0 | 11.08 | 3.29 ± 1.8 | 2.48 ± 0.5 | 4.62 ± 3.1 | 6.56 ± 1.2 | 7.01 ± 1.8 | 6.89 ± 0.6 |
| 10 | DW-190 | 19.36 ± 3.8 | 26.4 | 6.18 ± 3.4 | 11.01 ± 0.3 | 10.31 ± 1.0 | 7.16 ± 4.2 | 11.70 ± 0.6 | 11.64 ± 1.7 |
| 11 | DW-191 | 24.89 ± 6.8 | 26.97 | 8.39 ± 3.04 | 13.82 ± 4.6 | 15.66 ± 3.3 | 22.94 ± 0.5 | 18.33 ± 6.2 | 24.06 ± 2.4 |
| 12 | DW-199 | 14.27 | 10.23 | 3.75 ± 1.1 | 1.80 ± 0.7 | 4.33 ± 0.2 | 4.70 ± 1.1 | 6.31 ± 1.4 | 4.03 ± 1.7 |
| 13 | DW-271 | 2.96 ± 0.2 | 3.08 ± 0.06 | 2.99 ± 0.4 | 1.73 ± 0.55 | 2.77 ± 0.03 | 3.44 ± 1.58 | 3.38 ± 0.52 | 5.34 ± 0.62 |
| 14 | DW-288 | 4.90 ± 0.0 | 7.21 ± 2.9 | 3.48 ± 0.1 | 2.42 ± 0.21 | 9.52 ± 0.70 | 9.27 ± 2.12 | 4.28 ± 0.61 | 5.28 ± 1.15 |

Table 2 presents IC50 values for additional butenolide-based compounds evaluated against the same panel of cell lines. The data revealed structure-activity relationships among the tested compounds, with certain structural modifications resulting in improved cytotoxic potency or selectivity. Compounds demonstrating low IC50 values against both hormone-responsive and hormone-independent cancer cell lines suggested activity through mechanisms independent of hormone receptor status, indicating potential utility against cancers that are refractory to hormone-based therapies. The activity profiles across lung, prostate, liver, endothelial, and breast cancer cell lines demonstrated broad-spectrum anticancer potential for the butenolide scaffold.

TABLE 2
IC₅₀ values obtained based on MTT cell viability assay

| | | Cell Line and IC₅₀ Value (μM) | | | | | | | |
|---|---|---|---|---|---|---|---|---|---|
| | | | | | | | | MDA- | |
| | | | DU- | | | SK- | | MB- | |
| S/No. | Compound | A549 | 145 | LNCaP | HepG2 | HEP-1 | MCF-7 | 231 | Vero |
| 1 | DW-365 | 32.34 | 11.06 | 11.28 | 28.13 | 11.19 | 11.17 | 10.85 | 10.72 |
| 2 | DW-373 | 21.25 | 22.38 | 21.71 | 22.37 | 16.44 | 13.05 | 10.80 | 10.72 |
| 3 | DW-380 | 15.48 | 11.68 | 7.64 | 22.46 | 11.75 | 11.18 | 10.71 | 10.19 |
| 4 | DW-375 | 48.74 | 10.8 | 24.34 | 114.5 | 20.39 | 10.89 | 10.05 | 22.15 |
| 5 | DW-427 | 22.74 | 24.18 | 15.62 | 15.51 | 15.29 | 23.51 | 10.57 | 22.50 |
| 6 | DW-435 | 16.91 | 16.82 | 12.45 | 17.99 | 12.51 | 22.69 | 10.21 | 22.61 |
| 7 | DW-438 | 13.57 | 13.24 | 10.70 | 16.53 | 11.78 | 17.75 | 6.88 | 15.67 |

Table 3 presents IC₅₀ values for a further series of butenolide-based compounds evaluated using the PI-Triton X-100 cell viability assay. This assay employs Propidium Iodide (PI), a membrane-impermeant fluorescent dye that cannot enter cells with intact membranes, to detect dead cells. When a cell dies or has a compromised membrane, PI enters the cell, binds to DNA/RNA, and undergoes a significant fluorescence increase. Triton X-100, a nonionic surfactant, is used to permeabilize cells by disrupting cell membranes, allowing PI to enter all cells for total cell count and DNA quantification. The results from this series confirmed the cytotoxic potential of the butenolide scaffold and identified lead compounds with favorable activity profiles across multiple cancer types. Compounds exhibiting potent activity against MDA-MB-231 triple-negative breast cancer cells and Du145 hormone-independent prostate cancer cells were of particular interest given the limited treatment options available for these aggressive cancer subtypes. The selectivity indices calculated from the ratio of Vero IC₅₀ to cancer cell IC₅₀values provided a measure of the therapeutic window for each compound.

TABLE 3
IC₅₀ values obtained based on PI-Triton X-100 cell viability assay

Cell Line and IC₅₀ Value (μM)

| S/No. | Compound | MCF-7 | MDA-MB-231 | Vero |
|---|---|---|---|---|
| 2 | DW-749 | 4.37 | 1.64 | 1.87 |
| 3 | DW-759 | 5.18 | 11.25 | 10.66 |
| 4 | DW-782 | 7.42 | 5.46 | 6.69 |

The MTT and PI-Triton X-100 assay results collectively demonstrated that butenolide-based compounds possess significant cytotoxic activity against a broad range of cancer cell lines including lung, prostate, liver, endothelial, and breast cancer cells. The activity observed against hormone-independent cell lines such as MDA-MB-231 and Du145 indicated that the compounds exert anticancer effects through mechanisms beyond hormone receptor modulation. While PI staining identifies cells with lost membrane integrity, it does not distinguish between necrotic and apoptotic cell death mechanisms. The favorable selectivity profiles observed for several compounds, as evidenced by higher IC₅₀values against Vero control cells compared to cancer cells, suggested potential for therapeutic application with reduced toxicity to normal.

What is claimed is:

1. A compound of formula (I) having the following structure: 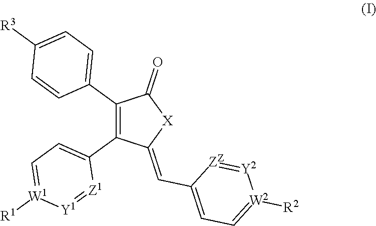
or a stereoisomer, pharmaceutically acceptable salt, oxide, or solvate thereof, wherein
R¹ is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶;
R² is optionally present, and when present is H, hydroxyl, —SO₂C₁₋₆ alkyl, —NR⁴R⁵, —CN, heterocyclyl, aryl, heteroaryl, —CH₂R⁶, —OR⁶ or —SR⁶;
R³ is H, hydroxyl, —SO₂C₁₋₆ alkyl, —NR⁴R⁵, —CN, heterocyclyl, aryl, heteroaryl, —CH₂R⁶, —OR⁶ or —SR⁶;
X is O or NR⁷;
W¹ is C or N, wherein when W¹ is N, R¹ is not present;
Y¹ is CH or N;
Z¹ is CH or N;
W² is C or N, wherein when W² is N, R² is not present;
Y² is CH or N;
Z² is CH or N;
R⁴ is H, C₁-C₆ alkyl, C₂-C₆ alkenyl, wherein C₁-C₆ alkyl and C₂-C₆ alkenyl can be substituted with —N(CH₃)₂ or heterocyclyl;
R⁵ is H or C₁-C₆ alkyl; or
R⁴ and R⁵ combine with the nitrogen atom to which they are attached to form a heterocyclic ring, wherein the heterocyclic ring can be substituted with C₁-C₆ alkyl;
R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and
R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.

2. The compound according to claim 1, selected from the group consisting of

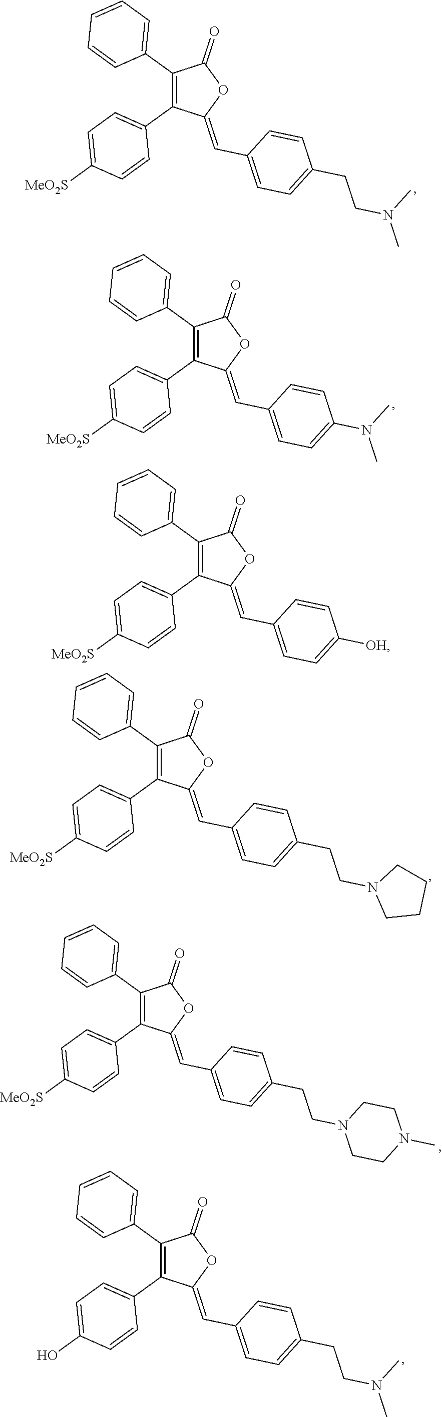

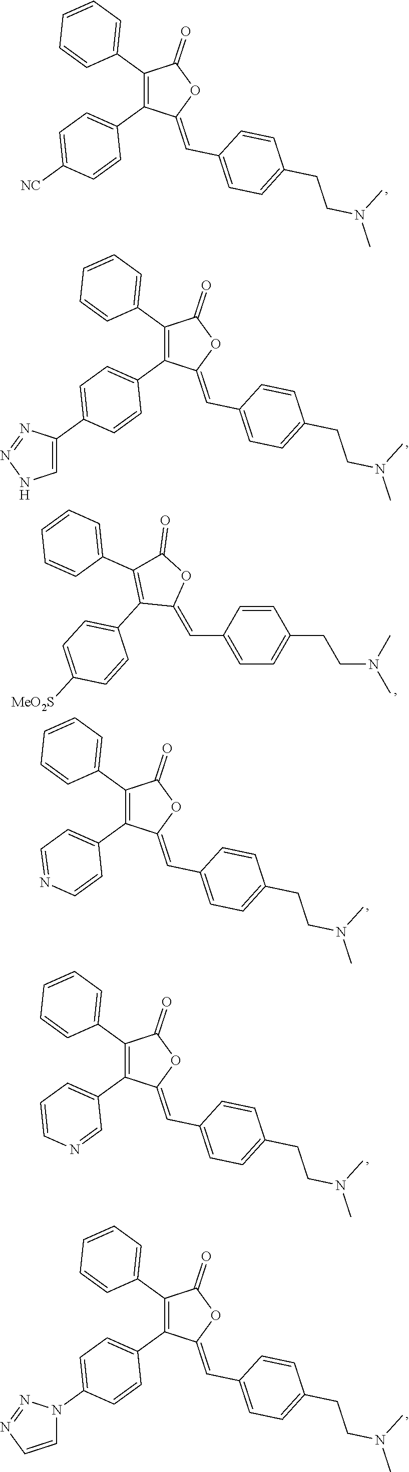

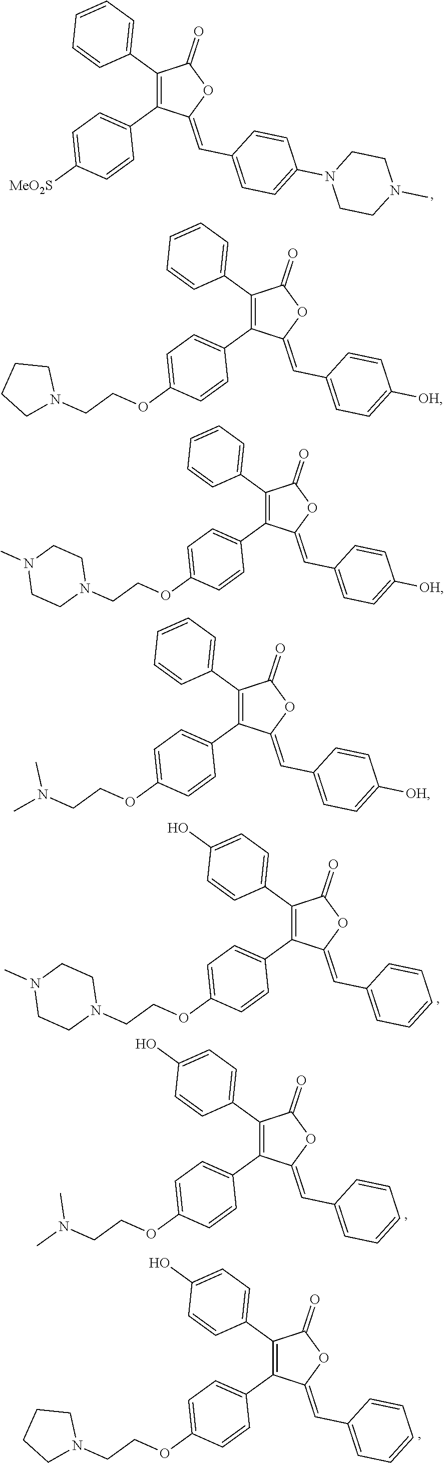

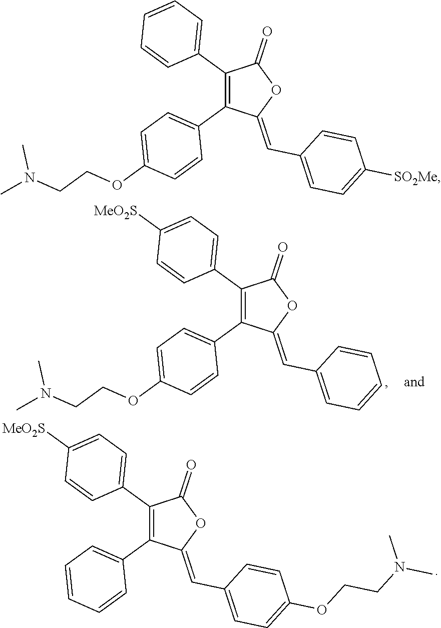

3. A compound of formula (I) having the following structure: 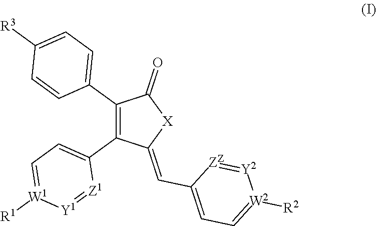
or a stereoisomer, pharmaceutically acceptable salt, oxide, or solvate thereof, wherein
R¹ is optionally present, and when present is H, hydroxyl, —SO₂C₁₋₆ alkyl, —NR⁴R⁵, —CN, heterocyclyl, aryl, heteroaryl, —OR⁶ or —SR⁶;
R² is optionally present, and when present is H, hydroxyl, —SO₂C₁₋₆ alkyl, —NR⁴R⁵, —CN, heterocyclyl, aryl, heteroaryl, —OR⁶ or —SR⁶;
R³ is H, hydroxyl, —SO₂C₁₋₆ alkyl, —NR⁴R⁵, —CN, heterocyclyl, aryl, heteroaryl, —OR⁶ or —SR⁶;
X is O or NR⁷;
W¹ is C or N, wherein when W¹ is N, R¹ is not present;
Y¹ is CH or N;
Z¹ is CH or N;
W² is C or N, wherein when W² is N, R² is not present;
Y² is CH or N;
Z² is CH or N;
R⁴ is H, C₁-C₆ alkyl, C₂-C₆ alkenyl, wherein C₁-C₆ alkyl and C₂-C₆ alkenyl can be substituted with —N(CH₃)₂ or heterocyclyl;
R⁵ is H or C₁-C₆ alkyl; or
R⁴ and R⁵ combine with the nitrogen atom to which they are attached to form a heterocyclic ring, wherein the heterocyclic ring can be substituted with C₁-C₆ alkyl;
R⁶ is C₁-C₆ alkyl or C₂-C₆ alkenyl, wherein C₁-C₆ alkyl and C₂-C₆ alkenyl can be substituted with —NR⁴R⁵, heteroaryl, or heterocyclyl; and
R⁷ is H, C₁-C₆ alkyl, or C₂-C₆ alkenyl, wherein C₁-C₆ alkyl and C₂-C₆ alkenyl can be substituted with —NR⁴R⁵, heteroaryl, or heterocyclyl.

4. The compound according to claim 3, selected from the group consisting of

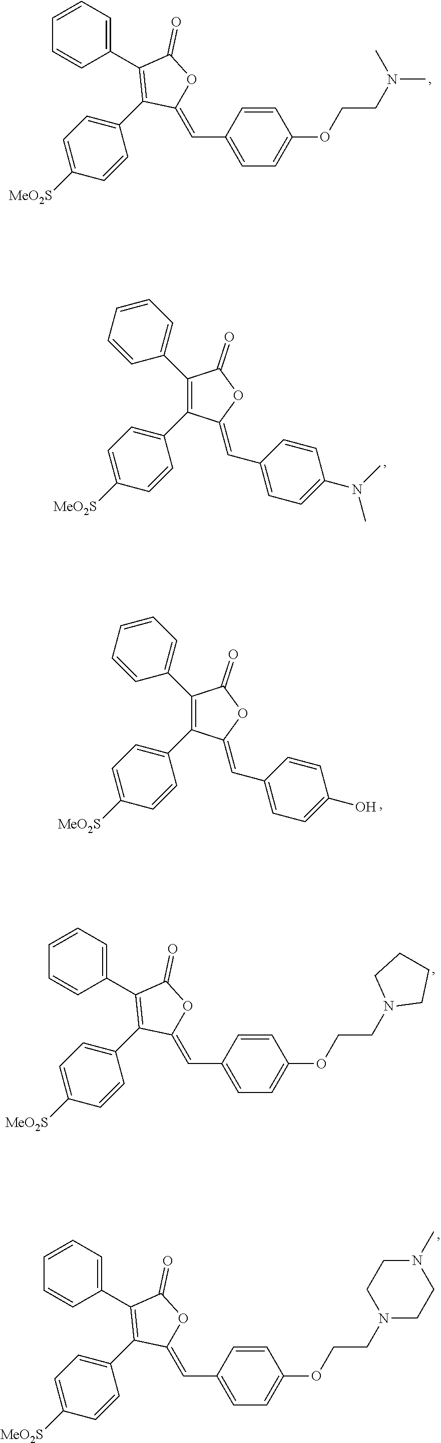 [SMILES: CN(C)CCOc1ccc(/C=C2\OC(=O)C(c3ccccc3)=C2c2ccc(S(C)(=O)=O)cc2)cc1 ; CN(C)c1ccc(/C=C2\OC(=O)C(c3ccccc3)=C2c2ccc(S(C)(=O)=O)cc2)cc1 ; CS(=O)(=O)c1ccc(C2=C(c3ccccc3)C(=O)O/C2=C\c2ccc(O)cc2)cc1 ; CS(=O)(=O)c1ccc(C2=C(c3ccccc3)C(=O)O/C2=C\c2ccc(OCCN3CCCC3)cc2)cc1 ; CN1CCN(CCOc2ccc(/C=C3\OC(=O)C(c4ccccc4)=C3c3ccc(S(C)(=O)=O)cc3)cc2)CC1]

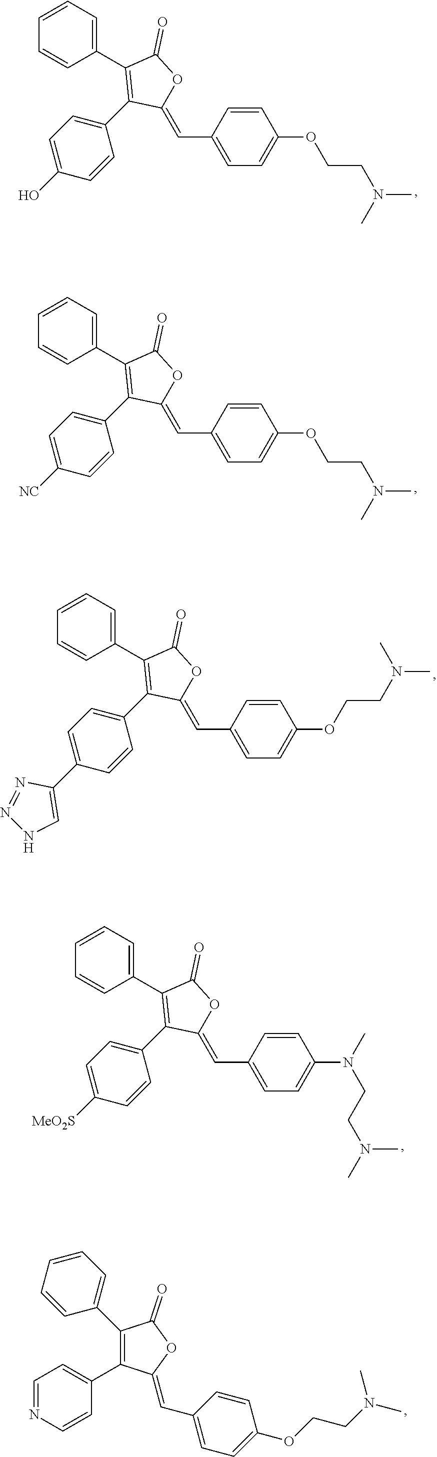 [SMILES: CN(C)CCOc1ccc(/C=C2\OC(=O)C(c3ccccc3)=C2c2ccc(O)cc2)cc1 ; CN(C)CCOc1ccc(/C=C2\OC(=O)C(c3ccccc3)=C2c2ccc(C#N)cc2)cc1 ; CN(C)CCOc1ccc(/C=C2\OC(=O)C(c3ccccc3)=C2c2ccc(-c3c[nH]nn3)cc2)cc1 ; CN(C)CCN(C)c1ccc(/C=C2\OC(=O)C(c3ccccc3)=C2c2ccc(S(C)(=O)=O)cc2)cc1 ; CN(C)CCOc1ccc(/C=C2\OC(=O)C(c3ccccc3)=C2c2ccncc2)cc1]

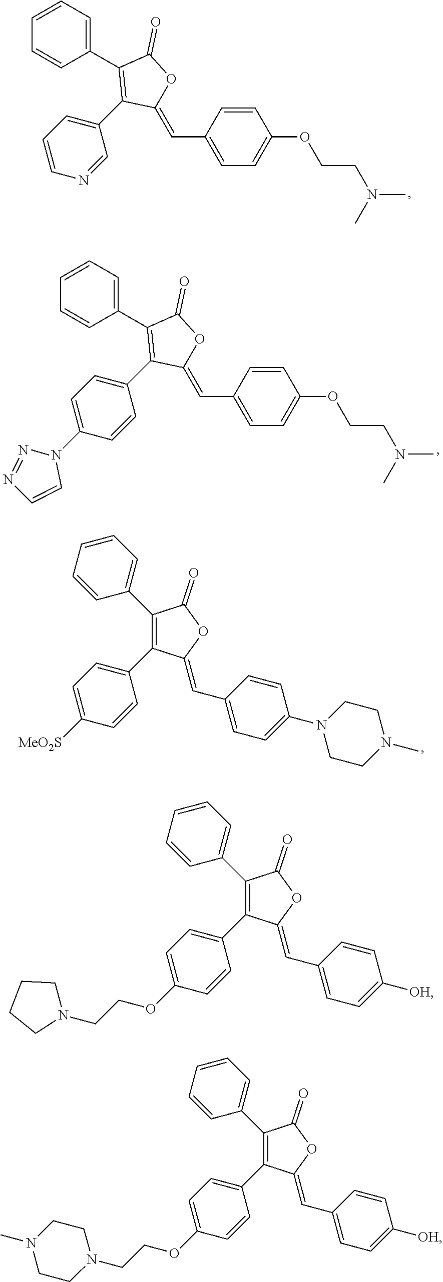

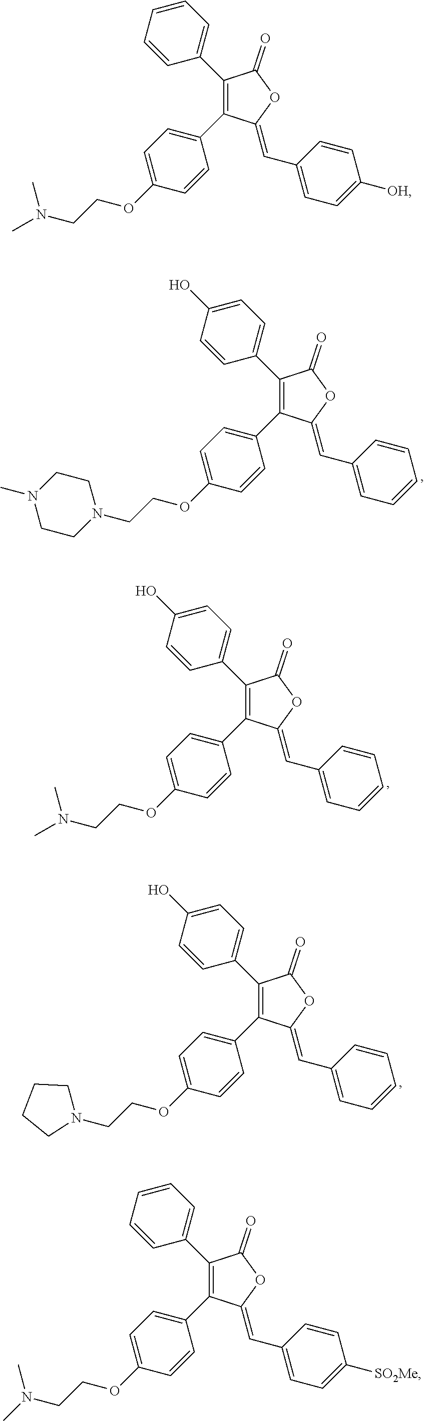 [SMILES: CN(C)CCOc1ccc(C2=C(c3ccccc3)C(=O)O/C2=C\c2ccc(O)cc2)cc1 ; CN1CCN(CCOc2ccc(C3=C(c4ccc(O)cc4)C(=O)O/C3=C\c3ccccc3)cc2)CC1 ; CN(C)CCOc1ccc(C2=C(c3ccc(O)cc3)C(=O)O/C2=C\c2ccccc2)cc1 ; O=C1O/C(=C\c2ccccc2)C(c2ccc(OCCN3CCCC3)cc2)=C1c1ccc(O)cc1 ; CN(C)CCOc1ccc(C2=C(c3ccccc3)C(=O)O/C2=C\c2ccc(S(C)(=O)=O)cc2)cc1]

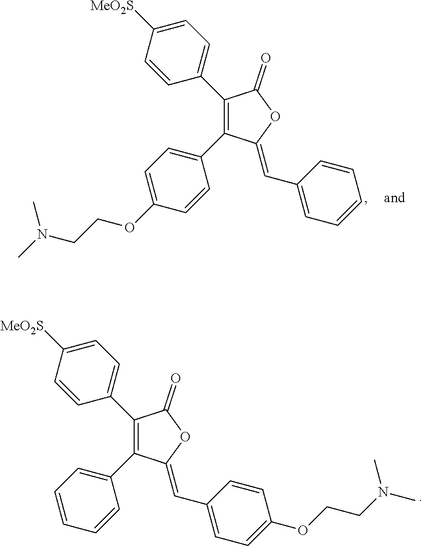 [SMILES: CN(C)CCOc1ccc(C2=C(c3ccc(S(C)(=O)=O)cc3)C(=O)O/C2=C\c2ccccc2)cc1 ; CN(C)CCOc1ccc(/C=C2\OC(=O)C(c3ccc(S(C)(=O)=O)cc3)=C2c2ccccc2)cc1]

5. A method of treating cancer in a subject, said method comprising:
providing a compound according to claim 1 to a subject in need of said treatment.

6. The method of claim 5, wherein the cancer is an estrogen receptor (ER) dependent cancer.

7. The method of claim 6, wherein the cancer is selected from the group consisting of breast cancer, prostate cancer, lung cancer, and liver cancer.

8. A method of treating cancer in a subject, said method comprising:
providing a compound according to claim 3 to a subject in need of said treatment.

9. The method of claim 8, wherein the cancer is an estrogen receptor (ER) dependent cancer.

10. The method of claim 9, wherein the cancer is selected from the group consisting of breast cancer, prostate cancer, lung cancer, and liver cancer.

11. A method of inhibiting an estrogen receptor alpha, said method comprising:
providing a compound according to claim 1 under conditions effective to inhibit the estrogen receptor alpha (ERα).

12. A method of inhibiting an estrogen receptor alpha, said method comprising:
providing a compound according to claim 3 under conditions effective to inhibit the estrogen receptor alpha (ERα).

13. A method of degrading an estrogen receptor alpha, said method comprising:
providing a compound according to claim 1 under conditions effective to degrade the estrogen receptor alpha (ERα).

14. A method of degrading an estrogen receptor alpha, said method comprising:
providing a compound according to claim 3 under conditions effective to degrade the estrogen receptor alpha (ERα).

15. A method of treating a hyperproliferative disorder, said method comprising:
providing a compound according to claim 1 under conditions effective to treat a hyperproliferative disorder.

16. The method of claim 15, wherein the hyperproliferative disorder depends on ERα, RET, GREB1, ADCY5 or TFF1.

17. The method of claim 15, wherein the hyperproliferative disorder is a cancer selected from the group consisting of breast cancer, prostate cancer, lung cancer, and liver cancer.

18. A method of treating a hyperproliferative disorder, said method comprising:
providing a compound according to claim 3 under conditions effective to treat a hyperproliferative disorder.

19. A composition comprising:
a compound according to claim 1; and
a carrier.

20. A composition comprising:
a compound according to claim 3; and
a carrier.
